SEQUENCE LISTING (375 sequences)

SEQ ID NO: 1 - protein, 4 aa, Artificial Sequence
  Description of Artificial Sequence: Synthetic peptide
FLPS

SEQ ID NO: 2 - protein, 12 aa, Artificial Sequence
  Description of Artificial Sequence: Synthetic peptide
FLPSEFGVDVDR

SEQ ID NO: 3 - protein, 14 aa, Artificial Sequence
  Description of Artificial Sequence: Synthetic peptide
KRFLPSEFGVDVDR

SEQ ID NO: 4 - protein, 14 aa, Artificial Sequence
  Description of Artificial Sequence: Synthetic peptide
KRFFPSEFGLDVDR

SEQ ID NO: 5 - protein, 14 aa, Artificial Sequence
  Description of Artificial Sequence: Synthetic peptide
KRFLPSEFGFDVDH

SEQ ID NO: 6 - protein, 14 aa, Artificial Sequence
  Description of Artificial Sequence: Synthetic peptide
KRFFPSEFGNDVDK

SEQ ID NO: 7 - protein, 14 aa, Artificial Sequence
  Description of Artificial Sequence: Synthetic peptide
KRFFPSEFGTDVDR

SEQ ID NO: 8 - protein, 14 aa, Artificial Sequence
  Description of Artificial Sequence: Synthetic peptide
KRFLPSEFGMDPPR

SEQ ID NO: 9 - protein, 14 aa, Artificial Sequence
  Description of Artificial Sequence: Synthetic peptide
KRFLPSEFGMDPAL

SEQ ID NO: 10 - protein, 14 aa, Artificial Sequence
  Description of Artificial Sequence: Synthetic peptide
RRFLPSEFGLDPDH

SEQ ID NO: 11 - protein, 14 aa, Artificial Sequence
  Description of Artificial Sequence: Synthetic peptide
KRFLPSEFGMDPDI

SEQ ID NO: 12 - protein, 14 aa, Artificial Sequence
  Description of Artificial Sequence: Synthetic peptide
KRFFPSEFGNDVDR

SEQ ID NO: 13 - protein, 14 aa, Artificial Sequence
  Description of Artificial Sequence: Synthetic peptide
KKFYPSEFGNDVDR

SEQ ID NO: 14 - protein, 15 aa, Artificial Sequence
  Description of Artificial Sequence: Synthetic peptide
VKRFFPSEFGLDVDR

SEQ ID NO: 15 - protein, 5 aa, Unknown
  Description of Unknown: [Leu]-enkephalin peptide
YGGFL

SEQ ID NO: 16 - protein, 5 aa, Unknown
  Description of Unknown: [Met]-enkephalin peptide
YGGFM

SEQ ID NO: 17 - protein, 10 aa, Unknown
  Description of Unknown: Alpha-Neoendorphin peptide
YGGFLRKYPK

SEQ ID NO: 18 - protein, 9 aa, Unknown
  Description of Unknown: Beta-Neoendorphin peptide
YGGPLRKYP

SEQ ID NO: 19 - protein, 31 aa, Homo sapiens
YGGFMTSEKSQTPLVTLFKNAIIKNAYKKGE

SEQ ID NO: 20 - protein, 16 aa, Homo sapiens
YGGFMTSEKSQTPLVT

SEQ ID NO: 21 - protein, 17 aa, Unknown
  Description of Unknown: Dynorphin A peptide
YGGFLRRIRPKLKWDNQ

SEQ ID NO: 22 - protein, 13 aa, Unknown
  Description of Unknown: Dynorphin B peptide
YGGFLRRQFKVVT

SEQ ID NO: 23 - protein, 4 aa, Unknown
  Description of Unknown: Endomorphin-1 peptide
YPTF

SEQ ID NO: 24 - protein, 4 aa, Unknown
  Description of Unknown: Endomorphin-2 peptide
YPFF

SEQ ID NO: 25 - protein, 5 aa, Artificial Sequence
  Description of Artificial Sequence: Synthetic peptide
  MOD_RES (2)..(2): D-Ala
  MOD_RES (4)..(4): N-Methyl-Phe
  MOD_RES (5)..(5): Gly-ol
YAGFG

SEQ ID NO: 26 - protein, 5 aa, Artificial Sequence
  Description of Artificial Sequence: Synthetic peptide
  MOD_RES (2)..(2): D-Pen
  MOD_RES (5)..(5): D-Pen
YXGFX

SEQ ID NO: 27 - protein, 5 aa, Artificial Sequence
  Description of Artificial Sequence: Synthetic peptide
  MOD_RES (2)..(2): D-Pen
  MOD_RES (4)..(4): D-Chloro-Phe
  MOD_RES (5)..(5): D-Pen
YXGFX

SEQ ID NO: 28 - protein, 5 aa, Artificial Sequence
  Description of Artificial Sequence: Synthetic peptide
  MOD_RES (2)..(2): D-Pen
  MOD_RES (4)..(5): Pen
YXGXX

SEQ ID NO: 29 - protein, 6 aa, Artificial Sequence
  Description of Artificial Sequence: Synthetic peptide
  MOD_RES (2)..(2): D-Ser
YSGFLT

SEQ ID NO: 30 - protein, 5 aa, Artificial Sequence
  Description of Artificial Sequence: Synthetic peptide
  MOD_RES (2)..(2): D-Ala
  MOD_RES (5)..(5): D-Leu
YAGFL

SEQ ID NO: 31 - protein, 7 aa, Artificial Sequence
  Description of Artificial Sequence: Synthetic peptide
YGGFMRF

SEQ ID NO: 32 - protein, 8 aa, Artificial Sequence
  Description of Artificial Sequence: Synthetic peptide
  MOD_RES (1)..(1): D-Phe
  MOD_RES (4)..(4): D-Trp
  MOD_RES (5)..(5): Orn
  MOD_RES (7)..(7): Pen
FCYWXTXT

SEQ ID NO: 33 - protein, 5 aa, Artificial Sequence
  Description of Artificial Sequence: Synthetic peptide
  N-terminus acetylated
RYRIK

SEQ ID NO: 34 - protein, 4 aa, Artificial Sequence
  Description of Artificial Sequence: Synthetic peptide
  MOD_RES (2)..(2): D-Arg
YRFK

SEQ ID NO: 35 - protein, 5 aa, Artificial Sequence
  Description of Artificial Sequence: Synthetic peptide
  MOD_RES (2)..(2): D-Ala
  MOD_RES (4)..(4): N-Methyl-Phe
  MOD_RES (5)..(5): Gly-ol
YAGFG

SEQ ID NO: 36 - protein, 6 aa, Artificial Sequence
  Description of Artificial Sequence: Synthetic peptide
  MOD_RES (2)..(2): D-Ala
  MOD_RES (5)..(5): D-Leu
  MOD_RES (6)..(6): D-Cys
YAGFLC

SEQ ID NO: 37 - protein, 8 aa, Artificial Sequence
  Description of Artificial Sequence: Synthetic peptide
  MOD_RES (2)..(2): D-Ala
  C-terminus amidated
YAFEVVPG

SEQ ID NO: 38 - protein, 7 aa, Artificial Sequence
  Description of Artificial Sequence: Synthetic peptide
  MOD_RES (2)..(2): D-Ala
YAFDVVG

SEQ ID NO: 39 - protein, 4 aa, Artificial Sequence
  Description of Artificial Sequence: Synthetic peptide
  MOD_RES (3)..(3): Methyl-Phe
  MOD_RES (4)..(4): D-Pro
YPFP

SEQ ID NO: 40 - protein, 5 aa, Artificial Sequence
  Description of Artificial Sequence: Synthetic peptide
  MOD_RES (1)..(1): N,N-diallyl-Tyr
  MOD_RES (2)..(3): Aib
YXXFL

SEQ ID NO: 41 - protein, 4 aa, Unknown
  Description of Unknown: Morphiceptin peptide
YPFP

SEQ ID NO: 42 - protein, 17 aa, Unknown
  Description of Unknown: Nociceptin orphanin FQ peptide
FGGFTGARKSARKLANQ

SEQ ID NO: 43 - protein, 18 aa, Unknown
  Description of Unknown: Nocistatin peptide
TEPGLEEVGEIEGQKQLQ

SEQ ID NO: 44 - protein, 18 aa, Homo sapiens
AGEGLNSQFWSLAAPQRF

SEQ ID NO: 45 - protein, 11 aa, Homo sapiens
SQAFLFQPQRF

SEQ ID NO: 46 - protein, 11 aa, Unknown
  Description of Unknown: Substrate P peptide
RPKPQQFFGLM

SEQ ID NO: 47 - protein, 8 aa, Homo sapiens [sequence omitted]

SEQ ID NO: 48 - protein, 7 aa, Bos sp.
YPFPGPI

SEQ ID NO: 49 - protein, 42 aa, Artificial Sequence [sequence omitted]

SEQ ID NO: 50 - protein, 6 aa, Artificial Sequence
  Description of Artificial Sequence: Synthetic peptide
NPFLPS

SEQ ID NO: 51 - protein, 6 aa, Artificial Sequence
  Description of Artificial Sequence: Synthetic peptide
NPYLPS

SEQ ID NO: 52 - protein, 6 aa, Artificial Sequence
  Description of Artificial Sequence: Synthetic peptide
NPWLPS

SEQ ID NO: 53 - protein, 6 aa, Artificial Sequence
  Description of Artificial Sequence: Synthetic peptide
NPHLPS

SEQ ID NO: 54 - protein, 6 aa, Artificial Sequence
  Description of Artificial Sequence: Synthetic peptide
DPFLPS

SEQ ID NO: 55 - protein, 6 aa, Artificial Sequence
  Description of Artificial Sequence: Synthetic peptide
DPYLPS

SEQ ID NO: 56 - protein, 6 aa, Artificial Sequence
  Description of Artificial Sequence: Synthetic peptide
DPWLPS

SEQ ID NO: 57 - protein, 6 aa, Artificial Sequence
  Description of Artificial Sequence: Synthetic peptide
DPHLPS

SEQ ID NO: 58 - protein, 8 aa, Artificial Sequence
  Description of Artificial Sequence: Synthetic peptide
FLPPPPSS

SEQ ID NO: 59 - protein, 8 aa, Artificial Sequence
  Description of Artificial Sequence: Synthetic peptide
YLPPPPSS

SEQ ID NO: 60 - protein, 8 aa, Artificial Sequence
  Description of Artificial Sequence: Synthetic peptide
WLPPPPSS

SEQ ID NO: 61 - protein, 8 aa, Artificial Sequence
  Description of Artificial Sequence: Synthetic peptide
HLPPPPSS

SEQ ID NO: 62 - protein, 6 aa, Artificial Sequence
  Description of Artificial Sequence: Synthetic peptide
GIPYTY

SEQ ID NO: 63 - protein, 6 aa, Artificial Sequence
  Description of Artificial Sequence: Synthetic peptide
SIPYTY

SEQ ID NO: 64 - protein, 6 aa, Artificial Sequence
  Description of Artificial Sequence: Synthetic peptide
TIPYTY

SEQ ID NO: 65 - protein, 6 aa, Artificial Sequence
  Description of Artificial Sequence: Synthetic peptide
GVPYTY

SEQ ID NO: 66 - protein, 6 aa, Artificial Sequence
  Description of Artificial Sequence: Synthetic peptide
GIPFTY

SEQ ID NO: 67 - protein, 6 aa, Artificial Sequence
  Description of Artificial Sequence: Synthetic peptide
NIPFTY

SEQ ID NO: 68 - protein, 6 aa, Artificial Sequence
  Description of Artificial Sequence: Synthetic peptide
GIPFTF

SEQ ID NO: 69 - protein, 6 aa, Artificial Sequence
  Description of Artificial Sequence: Synthetic peptide
GIPHTY

SEQ ID NO: 70 - protein, 8 aa, Artificial Sequence
  Description of Artificial Sequence: Synthetic peptide
YTIKAVDD

SEQ ID NO: 71 - protein, 8 aa, Artificial Sequence
  Description of Artificial Sequence: Synthetic peptide
YTINAVDD

SEQ ID NO: 72 - protein, 8 aa, Artificial Sequence
  Description of Artificial Sequence: Synthetic peptide
YTIEAVDD

SEQ ID NO: 73 - protein, 8 aa, Artificial Sequence
  Description of Artificial Sequence: Synthetic peptide
YTINSVDD

SEQ ID NO: 74 - protein, 8 aa, Artificial Sequence
  Description of Artificial Sequence: Synthetic peptide
YTIRAAND

SEQ ID NO: 75 - protein, 8 aa, Artificial Sequence
  Description of Artificial Sequence: Synthetic peptide
YTIKTIDD

SEQ ID NO: 76 - protein, 8 aa, Artificial Sequence
  Description of Artificial Sequence: Synthetic peptide
FTIKAAND

SEQ ID NO: 77 - protein, 8 aa, Artificial Sequence
  Description of Artificial Sequence: Synthetic peptide
FTIKAVDD

SEQ ID NO: 78 - protein, 16 aa, Artificial Sequence
  Description of Artificial Sequence: Synthetic peptide
SFGVEASELYPDVKYT

SEQ ID NO: 79 - protein, 16 aa, Artificial Sequence
  Description of Artificial Sequence: Synthetic peptide
SWGLEASELYPDVKYT

SEQ ID NO: 80 - protein, 16 aa, Artificial Sequence
  Description of Artificial Sequence: Synthetic peptide
SFGVEATALYPDVKYT

SEQ ID NO: 81 - protein, 7 aa, Artificial Sequence
  Description of Artificial Sequence: Synthetic peptide
HAVFVNG

SEQ ID NO: 82 - protein, 7 aa, Artificial Sequence
  Description of Artificial Sequence: Synthetic peptide
HCVFVKG

SEQ ID NO: 83 - protein, 7 aa, Artificial Sequence
  Description of Artificial Sequence: Synthetic peptide
HSVFVNG

SEQ ID NO: 84 - protein, 7 aa, Artificial Sequence
  Description of Artificial Sequence: Synthetic peptide
HSAFVKG

SEQ ID NO: 85 - protein, 7 aa, Artificial Sequence
  Description of Artificial Sequence: Synthetic peptide
HAAFVRG

SEQ ID NO: 86 - protein, 7 aa, Artificial Sequence
  Description of Artificial Sequence: Synthetic peptide
HAGYIRG

SEQ ID NO: 87 - protein, 7 aa, Artificial Sequence
  Description of Artificial Sequence: Synthetic peptide
HTAFVKG

SEQ ID NO: 88 - protein, 7 aa, Artificial Sequence
  Description of Artificial Sequence: Synthetic peptide
HSSYVKG

SEQ ID NO: 89 - skipped

SEQ ID NO: 90 - protein, 12 aa, Artificial Sequence
  Description of Artificial Sequence: Synthetic peptide
DFTSFTIDPSFG

SEQ ID NO: 91 - protein, 16 aa, Artificial Sequence
  Description of Artificial Sequence: Synthetic peptide
CFAGLFLFVPCLGGCH

SEQ ID NO: 92 - protein, 16 aa, Artificial Sequence
  Description of Artificial Sequence: Synthetic peptide
HCGGLCPVFLFLGAFC

SEQ ID NO: 93 - protein, 8 aa, Artificial Sequence
  Description of Artificial Sequence: Synthetic peptide
GAFYAAFC

SEQ ID NO: 94 - protein, 12 aa, Artificial Sequence
  Description of Artificial Sequence: Synthetic peptide
GFSPDITFSTFD

SEQ ID NO: 95 - protein, 8 aa, Artificial Sequence
  Description of Artificial Sequence: Synthetic peptide
TLFYGAFC

SEQ ID NO: 96 - protein, 8 aa, Artificial Sequence
  Description of Artificial Sequence: Synthetic peptide
PLSYGAFF

SEQ ID NO: 97 - protein, 8 aa, Artificial Sequence
  Description of Artificial Sequence: Synthetic peptide
PLSYGGFY

SEQ ID NO: 98 - protein, 8 aa, Artificial Sequence
  Description of Artificial Sequence: Synthetic peptide
GAFYGAFM

SEQ ID NO: 99 - protein, 8 aa, Artificial Sequence
  Description of Artificial Sequence: Synthetic peptide
FFAGYFLP

SEQ ID NO: 100 - protein, 8 aa, Artificial Sequence
  Description of Artificial Sequence: Synthetic peptide
CFGGYYLP

SEQ ID NO: 101 - protein, 8 aa, Artificial Sequence
  Description of Artificial Sequence: Synthetic peptide
CFAAYFAG

SEQ ID NO: 102 - protein, 8 aa, Artificial Sequence
  Description of Artificial Sequence: Synthetic peptide
CFAGYFLT

SEQ ID NO: 103 - protein, 8 aa, Artificial Sequence
  Description of Artificial Sequence: Synthetic peptide
FFAGYSLP

SEQ ID NO: 104 - protein, 8 aa, Artificial Sequence
  Description of Artificial Sequence: Synthetic peptide
YFGGYSLP

SEQ ID NO: 105 - protein, 8 aa, Artificial Sequence
  Description of Artificial Sequence: Synthetic peptide
MFAGYFAG

SEQ ID NO: 106 - protein, 8 aa, Artificial Sequence
  Description of Artificial Sequence: Synthetic peptide
YFAGYALP

SEQ ID NO: 107 - protein, 8 aa, Artificial Sequence
  Description of Artificial Sequence: Synthetic peptide
WFADFFLP

SEQ ID NO: 108 - protein, 8 aa, Artificial Sequence
  Description of Artificial Sequence: Synthetic peptide
PLFYGAFF

SEQ ID NO: 109 - protein, 8 aa, Artificial Sequence
  Description of Artificial Sequence: Synthetic peptide
PLYYGGFC

SEQ ID NO: 110 - protein, 9 aa, Artificial Sequence
  Description of Artificial Sequence: Synthetic peptide
TGYIGKFLV

SEQ ID NO: 111 - protein, 9 aa, Artificial Sequence
  Description of Artificial Sequence: Synthetic peptide
TGYIGKFIV

SEQ ID NO: 112 - protein, 9 aa, Artificial Sequence
  Description of Artificial Sequence: Synthetic peptide
TGYIGKFVA

SEQ ID NO: 113 - protein, 9 aa, Artificial Sequence
  Description of Artificial Sequence: Synthetic peptide
TGYIGKYIV

SEQ ID NO: 114 - protein, 9 aa, Artificial Sequence
  Description of Artificial Sequence: Synthetic peptide
TGYIGKYLV

SEQ ID NO: 115 - protein, 8 aa, Artificial Sequence
  Description of Artificial Sequence: Synthetic peptide
TGYIGRHV

SEQ ID NO: 116 - protein, 8 aa, Artificial Sequence
  Description of Artificial Sequence: Synthetic peptide
TGYLGRHV

SEQ ID NO: 117 - protein, 9 aa, Artificial Sequence
  Description of Artificial Sequence: Synthetic peptide
TGYIGKRIV

SEQ ID NO: 118 - protein, 9 aa, Artificial Sequence
  Description of Artificial Sequence: Synthetic peptide
TGFIGKRIV

SEQ ID NO: 119 - protein, 9 aa, Artificial Sequence
  Description of Artificial Sequence: Synthetic peptide
VLFKGIYGT

SEQ ID NO: 120 - protein, 9 aa, Artificial Sequence
  Description of Artificial Sequence: Synthetic peptide
VIFKGIYGT

SEQ ID NO: 121 - protein, 9 aa, Artificial Sequence
  Description of Artificial Sequence: Synthetic peptide
AVFKGIYGT

SEQ ID NO: 122 - protein, 9 aa, Artificial Sequence
  Description of Artificial Sequence: Synthetic peptide
VIYKGIYGT

SEQ ID NO: 123 - protein, 9 aa, Artificial Sequence
  Description of Artificial Sequence: Synthetic peptide
VLYKGIYGT

SEQ ID NO: 124 - protein, 8 aa, Artificial Sequence
  Description of Artificial Sequence: Synthetic peptide
VHRGIYGT

SEQ ID NO: 125 - protein, 8 aa, Artificial Sequence
  Description of Artificial Sequence: Synthetic peptide
VHRGLYGT

SEQ ID NO: 126 - protein, 16 aa, Artificial Sequence
  Description of Artificial Sequence: Synthetic peptide
TYKVDPYLESAEVGFS

SEQ ID NO: 127 - protein, 16 aa, Artificial Sequence
  Description of Artificial Sequence: Synthetic peptide
TYKVDPYLESAELGWS

SEQ ID NO: 128 - protein, 16 aa, Artificial Sequence
  Description of Artificial Sequence: Synthetic peptide
TYKVDPYLATAEVGFS

SEQ ID NO: 129 - protein, 16 aa, Artificial Sequence
  Description of Artificial Sequence: Synthetic peptide
TYKVDPYLESAEVGFS

SEQ ID NO: 130 - protein, 16 aa, Artificial Sequence
  Description of Artificial Sequence: Synthetic peptide
TYKVDPYLESAELGWS

SEQ ID NO: 131 - protein, 16 aa, Artificial Sequence
  Description of Artificial Sequence: Synthetic peptide
TYKVDPYLATAEVGFS

SEQ ID NO: 132 - protein, 6 aa, Artificial Sequence
  Description of Artificial Sequence: Synthetic peptide
KIYLIK

SEQ ID NO: 133 - protein, 6 aa, Artificial Sequence
  Description of Artificial Sequence: Synthetic peptide
KIYLVK

SEQ ID NO: 134 - protein, 6 aa, Artificial Sequence
  Description of Artificial Sequence: Synthetic peptide
RIYLIK

SEQ ID NO: 135 - protein, 6 aa, Artificial Sequence
  Description of Artificial Sequence: Synthetic peptide
RIYLVK

SEQ ID NO: 136 - protein, 6 aa, Artificial Sequence
  Description of Artificial Sequence: Synthetic peptide
KILYIK

SEQ ID NO: 137 - protein, 6 aa, Artificial Sequence
  Description of Artificial Sequence: Synthetic peptide
KVLYIK

SEQ ID NO: 138 - protein, 6 aa, Artificial Sequence
  Description of Artificial Sequence: Synthetic peptide
KVLYIR

SEQ ID NO: 139 - protein, 6 aa, Artificial Sequence
  Description of Artificial Sequence: Synthetic peptide
IKKEWL

SEQ ID NO: 140 - protein, 6 aa, Artificial Sequence
  Description of Artificial Sequence: Synthetic peptide
IKNEWL

SEQ ID NO: 141 - protein, 6 aa, Artificial Sequence
  Description of Artificial Sequence: Synthetic peptide
TKKEWL

SEQ ID NO: 142 - protein, 5 aa, Artificial Sequence
  Description of Artificial Sequence: Synthetic peptide
LKEWI

SEQ ID NO: 143 - protein, 6 aa, Artificial Sequence
  Description of Artificial Sequence: Synthetic peptide
IKDEWL

SEQ ID NO: 144 - protein, 6 aa, Artificial Sequence
  Description of Artificial Sequence: Synthetic peptide
IKGEWL

SEQ ID NO: 145 - protein, 6 aa, Artificial Sequence
  Description of Artificial Sequence: Synthetic peptide
ILEEWK

SEQ ID NO: 146 - protein, 6 aa, Artificial Sequence
  Description of Artificial Sequence: Synthetic peptide
LWEKKI

SEQ ID NO: 147 - protein, 6 aa, Artificial Sequence
  Description of Artificial Sequence: Synthetic peptide
LWENKI

SEQ ID NO: 148 - protein, 6 aa, Artificial Sequence
  Description of Artificial Sequence: Synthetic peptide
LWEKKT

SEQ ID NO: 149 - protein, 5 aa, Artificial Sequence
  Description of Artificial Sequence: Synthetic peptide
IWEKL

SEQ ID NO: 150 - protein, 6 aa, Artificial Sequence
  Description of Artificial Sequence: Synthetic peptide
LWEDKI

SEQ ID NO: 151 - protein, 6 aa, Artificial Sequence
  Description of Artificial Sequence: Synthetic peptide
LWEGKI

SEQ ID NO: 152 - protein, 6 aa, Artificial Sequence
  Description of Artificial Sequence: Synthetic peptide
KWEELI

SEQ ID NO: 153 - protein, 4 aa, Artificial Sequence
  Description of Artificial Sequence: Synthetic peptide
SPLF

SEQ ID NO: 154 - protein, 14 aa, Artificial Sequence
  Description of Artificial Sequence: Synthetic peptide
RDVDVGFESPLFRK

SEQ ID NO: 155 - protein, 4 aa, Artificial Sequence
  Description of Artificial Sequence: Synthetic peptide
PFPY

SEQ ID NO: 156 - protein, 6 aa, Artificial Sequence
  Description of Artificial Sequence: Synthetic peptide
SPLFPN

SEQ ID NO: 157 - protein, 6 aa, Artificial Sequence
  Description of Artificial Sequence: Synthetic peptide
SPLYPN

SEQ ID NO: 158 - protein, 6 aa, Artificial Sequence
  Description of Artificial Sequence: Synthetic peptide
SPLWPN

SEQ ID NO: 159 - protein, 6 aa, Artificial Sequence
  Description of Artificial Sequence: Synthetic peptide
SPLHPN

SEQ ID NO: 160 - protein, 6 aa, Artificial Sequence
  Description of Artificial Sequence: Synthetic peptide
SPLFPD

SEQ ID NO: 161 - protein, 6 aa, Artificial Sequence
  Description of Artificial Sequence: Synthetic peptide
SPLYPD

SEQ ID NO: 162 - protein, 6 aa, Artificial Sequence
  Description of Artificial Sequence: Synthetic peptide
SPLWPD

SEQ ID NO: 163 - protein, 6 aa, Artificial Sequence
  Description of Artificial Sequence: Synthetic peptide
SPLHPD

SEQ ID NO: 164 - protein, 8 aa, Artificial Sequence
  Description of Artificial Sequence: Synthetic peptide
SSPPPPLF

SEQ ID NO: 165 - protein, 8 aa, Artificial Sequence
  Description of Artificial Sequence: Synthetic peptide
SSPPPPLY

SEQ ID NO: 166 - protein, 8 aa, Artificial Sequence
  Description of Artificial Sequence: Synthetic peptide
SSPPPPLW

SEQ ID NO: 167 - protein, 8 aa, Artificial Sequence
  Description of Artificial Sequence: Synthetic peptide
SSPPPPLH

SEQ ID NO: 168 - protein, 6 aa, Artificial Sequence
  Description of Artificial Sequence: Synthetic peptide
GTYPIG

SEQ ID NO: 169 - protein, 6 aa, Artificial Sequence
  Description of Artificial Sequence: Synthetic peptide
YTYPIS

SEQ ID NO: 170 - protein, 6 aa, Artificial Sequence
  Description of Artificial Sequence: Synthetic peptide
YTYPIT

SEQ ID NO: 171 - protein, 6 aa, Artificial Sequence
  Description of Artificial Sequence: Synthetic peptide
YTYPVG

SEQ ID NO: 172 - protein, 6 aa, Artificial Sequence
  Description of Artificial Sequence: Synthetic peptide
YTFPIG

SEQ ID NO: 173 - protein, 6 aa, Artificial Sequence
  Description of Artificial Sequence: Synthetic peptide
YTFPIN

SEQ ID NO: 174 - protein, 6 aa, Artificial Sequence
  Description of Artificial Sequence: Synthetic peptide
FTFPIG

SEQ ID NO: 175 - protein, 6 aa, Artificial Sequence
  Description of Artificial Sequence: Synthetic peptide
YTHPIG

SEQ ID NO: 176 - protein, 8 aa, Artificial Sequence
  Description of Artificial Sequence: Synthetic peptide
DDVAKITY

SEQ ID NO: 177 - protein, 8 aa, Artificial Sequence
  Description of Artificial Sequence: Synthetic peptide
DDVANITY

SEQ ID NO: 178 - protein, 8 aa, Artificial Sequence
  Description of Artificial Sequence: Synthetic peptide
DDVAEITY

SEQ ID NO: 179 - protein, 8 aa, Artificial Sequence
  Description of Artificial Sequence: Synthetic peptide
DDVSNITY

SEQ ID NO: 180 - protein, 8 aa, Artificial Sequence
  Description of Artificial Sequence: Synthetic peptide
DNAARITY

SEQ ID NO: 181 - protein, 8 aa, Artificial Sequence
  Description of Artificial Sequence: Synthetic peptide
DDITKITY

SEQ ID NO: 182 - protein, 8 aa, Artificial Sequence
  Description of Artificial Sequence: Synthetic peptide
DNAAKITF

SEQ ID NO: 183 - protein, 7 aa, Artificial Sequence
  Description of Artificial Sequence: Synthetic peptide
GNVFVAH

SEQ ID NO: 184 - protein, 7 aa, Artificial Sequence
  Description of Artificial Sequence: Synthetic peptide
GKVFVCH

SEQ ID NO: 185 - protein, 7 aa, Artificial Sequence
  Description of Artificial Sequence: Synthetic peptide
GNVFVSH

SEQ ID NO: 186 - protein, 7 aa, Artificial Sequence
  Description of Artificial Sequence: Synthetic peptide
GKVFASH

SEQ ID NO: 187 - protein, 6 aa, Artificial Sequence
  Description of Artificial Sequence: Synthetic peptide
GVFAAH

SEQ ID NO: 188 - protein, 7 aa, Artificial Sequence
  Description of Artificial Sequence: Synthetic peptide
GRIYGAH

SEQ ID NO: 189 - protein, 7 aa, Artificial Sequence
  Description of Artificial Sequence: Synthetic peptide
GKVFATH

SEQ ID NO: 190 - protein, 7 aa, Artificial Sequence
  Description of Artificial Sequence: Synthetic peptide
GKVYSSH

SEQ ID NO: 191 - protein, 7 aa, Artificial Sequence
  Description of Artificial Sequence: Synthetic peptide
GKVYSSH

SEQ ID NO: 192 - protein, 7 aa, Artificial Sequence
  Description of Artificial Sequence: Synthetic peptide
CFAYFAG

SEQ ID NO: 193-196 - skipped (4 entries)

SEQ ID NO: 197 - protein, 8 aa, Artificial Sequence
  Description of Artificial Sequence: Synthetic peptide
PLFYGAFF

SEQ ID NO: 198 - protein, 8 aa, Artificial Sequence
  Description of Artificial Sequence: Synthetic peptide
PLYYGGFC

SEQ ID NO: 199 - protein, 8 aa, Artificial Sequence
  Description of Artificial Sequence: Synthetic peptide
GAFYAAFC

SEQ ID NO: 200-203 - skipped (4 entries)

SEQ ID NO: 204 - protein, 8 aa, Artificial Sequence
  Description of Artificial Sequence: Synthetic peptide
PLAYGAFY

SEQ ID NO: 205 - protein, 8 aa, Artificial Sequence
  Description of Artificial Sequence: Synthetic peptide
PLFFDAFW

SEQ ID NO: 206 - protein, 8 aa, Artificial Sequence
  Description of Artificial Sequence: Synthetic peptide
FFAGYFPL

SEQ ID NO: 207 - skipped

SEQ ID NO: 208 - protein, 9 aa, Artificial Sequence
  Description of Artificial Sequence: Synthetic peptide
VLFKGIYGT

SEQ ID NO: 209 - protein, 9 aa, Artificial Sequence
  Description of Artificial Sequence: Synthetic peptide
VIFKGIYGT

SEQ ID NO: 210 - protein, 9 aa, Artificial Sequence
  Description of Artificial Sequence: Synthetic peptide
AVFKGIYTG

SEQ ID NO: 211 - protein, 9 aa, Artificial Sequence
  Description of Artificial Sequence: Synthetic peptide
VIYKGIYGT

SEQ ID NO: 212 - protein, 9 aa, Artificial Sequence
  Description of Artificial Sequence: Synthetic peptide
VLYKGIYGT

SEQ ID NO: 213 - protein, 8 aa, Artificial Sequence
  Description of Artificial Sequence: Synthetic peptide
VHRGIYGT

SEQ ID NO: 214 - protein, 8 aa, Artificial Sequence
  Description of Artificial Sequence: Synthetic peptide
VHRGLYGT

SEQ ID NO: 215 - protein, 9 aa, Artificial Sequence
  Description of Artificial Sequence: Synthetic peptide
VIRKGIYGT

SEQ ID NO: 216 - protein, 9 aa, Artificial Sequence
  Description of Artificial Sequence: Synthetic peptide
VIRKGIFGT

SEQ ID NO: 217 - protein, 9 aa, Artificial Sequence
  Description of Artificial Sequence: Synthetic peptide
TGYIGKFLV

SEQ ID NO: 218 - protein, 9 aa, Artificial Sequence
  Description of Artificial Sequence: Synthetic peptide
TGYIGKFIV

SEQ ID NO: 219 - protein, 9 aa, Artificial Sequence
  Description of Artificial Sequence: Synthetic peptide
TGYIGKFVA

SEQ ID NO: 220 - protein, 9 aa, Artificial Sequence
  Description of Artificial Sequence: Synthetic peptide
TGYIGKYIV

SEQ ID NO: 221 - protein, 9 aa, Artificial Sequence
  Description of Artificial Sequence: Synthetic peptide
TGYIGKYLV

SEQ ID NO: 222 - protein, 8 aa, Artificial Sequence
  Description of Artificial Sequence: Synthetic peptide
TGYIGRHV

SEQ ID NO: 223 - protein, 8 aa, Artificial Sequence
  Description of Artificial Sequence: Synthetic peptide
TGYLGRHV

SEQ ID NO: 224 - protein, 6 aa, Artificial Sequence
  Description of Artificial Sequence: Synthetic peptide
KILYIK

SEQ ID NO: 225 - protein, 6 aa, Artificial Sequence
  Description of Artificial Sequence: Synthetic peptide
KVLYIK

SEQ ID NO: 226 - protein, 6 aa, Artificial Sequence
  Description of Artificial Sequence: Synthetic peptide
KILYIR

SEQ ID NO: 227 - protein, 6 aa, Artificial Sequence
  Description of Artificial Sequence: Synthetic peptide
KVLYIR

SEQ ID NO: 228 - protein, 6 aa, Artificial Sequence
  Description of Artificial Sequence: Synthetic peptide
KIYLIR

SEQ ID NO: 229 - protein, 6 aa, Artificial Sequence
  Description of Artificial Sequence: Synthetic peptide
KIYLVK

SEQ ID NO: 230 - protein, 6 aa, Artificial Sequence
  Description of Artificial Sequence: Synthetic peptide
RIYLVK

SEQ ID NO: 231 - protein, 6 aa, Artificial Sequence
  Description of Artificial Sequence: Synthetic peptide
LWEKKI

SEQ ID NO: 232 - protein, 6 aa, Artificial Sequence
  Description of Artificial Sequence: Synthetic peptide
LWENKI

SEQ ID NO: 233 - protein, 6 aa, Artificial Sequence
  Description of Artificial Sequence: Synthetic peptide
LWEKKT

SEQ ID NO: 234 - protein, 5 aa, Artificial Sequence
  Description of Artificial Sequence: Synthetic peptide
IWEKL

SEQ ID NO: 235 - protein, 6 aa, Artificial Sequence
  Description of Artificial Sequence: Synthetic peptide
LWEDKI

SEQ ID NO: 236 - protein, 6 aa, Artificial Sequence
  Description of Artificial Sequence: Synthetic peptide
LWEGKI

SEQ ID NO: 237 - protein, 6 aa, Artificial Sequence
  Description of Artificial Sequence: Synthetic peptide
KWEELI

SEQ ID NO: 238 - protein, 6 aa, Artificial Sequence
  Description of Artificial Sequence: Synthetic peptide
IKKEWL

SEQ ID NO: 239 - protein, 6 aa, Artificial Sequence
  Description of Artificial Sequence: Synthetic peptide
IKNEWL

SEQ ID NO: 240 - protein, 5 aa, Artificial Sequence
  Description of Artificial Sequence: Synthetic peptide
TKKEL

SEQ ID NO: 241 - protein, 5 aa, Artificial Sequence
  Description of Artificial Sequence: Synthetic peptide
LKEKL

SEQ ID NO: 242 - protein, 6 aa, Artificial Sequence
  Description of Artificial Sequence: Synthetic peptide
IKDEWL

SEQ ID NO: 243 - protein, 6 aa, Artificial Sequence
  Description of Artificial Sequence: Synthetic peptide
IKGEWL

SEQ ID NO: 244 - protein, 6 aa, Artificial Sequence
  Description of Artificial Sequence: Synthetic peptide
ILEEWK

SEQ ID NO: 245 - protein, 314 aa, Artificial Sequence
  Description of Artificial Sequence: Synthetic polypeptide
MDKKSRVLIVGGTGFIGKRIVKASLALGHPTYVLFRPEALSYIDKVQMLISFKQLGAKLLEASLDDHQGLVDVVKQVDVVISAVSGGLVRHHILDQLKLVEAIKEAGNIKRFLPSEFGMDPDVVEDPLEPGNITFIDKRKVRRAIEAATIPYTYVSSNMFAGFFAGSLAQLQDAPRMMPARDKVLIYGDGNVKGVYVDEDDAGIYIVKSIDDPRTLNKTVYIRPPMNILSQKEVVEIWERLSGLSLEKIYVSEDQLLNMKDKSYVEKMARCHLYHFFIKGDLYNFEIGPNATEGTKLYPEVKYTTMDSYMERYL

SEQ ID NO: 246 - protein, 316 aa, Artificial Sequence
  Description of Artificial Sequence: Synthetic polypeptide
MGESKRTEKTRVLVVGATGYIGKRIVRACLAEGHETYVLQRPEIGLEIEKVQLFLSFKKLGARIVEGSFSDHQSLVSAVKLVDVVVSAMSGVHFRSHNILVQLKLVEAIKEAGNVKRFLPSEFGMDPPRMGHALPPGRETFDQKMERQAIEAAGIPYTYVVGACFAAYFAGNLSQMVTLLPPKEKVNIYGDGNVKVVFADEDDIAKYTAKTLNDPRTLNKTVNIRPPDNVLTQLELVQIWEKLTGKELEKTNIAAQDFLANIEQMEIPHQAGIGHFYHIFYEGCLTDHEVGEDEEASSLYPDVKYKRMDDYLRMFL

SEQ ID NO: 247 - protein, 310 aa, Artificial Sequence
  Description of Artificial Sequence: Synthetic polypeptide
MATEKSKILVIGGTGYIGKFLVEASAKAGHSTFALVREATLSDPVKGKTVQSFKDLGVTILHGDLNDHESLVKAIKQVDVVISTVGSMQILDQTKIISAIKEAGNVKRFLPSEFGVDVDRTSAVEPAKSAFAGKIQIRRTIEAEGIPYTYAVTGCFGGYYLPTLVQFEPGLTSPPRDKVTILGDGNAKAVINKEEDIAAYTIKAVDDPRTLNKILYIKPSNNTLSMNEIVTLWEKKIGKSLEKTHLPEEQLLKSIQESPIPINVVLSINHAVFVNGDTNISIEPSFGVEASELYPDVKYTSVDEYLSYFA

SEQ ID NO: 248 - protein, 13 aa, Unknown
  Description of Unknown: Rimorphin peptide
YGGFLRRQFKVVT

SEQ ID NO: 249 - protein, 74 aa, Homo sapiens
KYPKRSSEVAGEGDGDSMGHEDLYKRYGGFLRRIRPKLKWDNQKRYGGFLRRQFKVVTRSQEDPNAYSGELFDA

SEQ ID NO: 250 - protein, 253 aa, Artificial Sequence
  Description of Artificial Sequence: Synthetic polypeptide
MAWQGLVLAACLLMFPSTTADCLSRCSLCAVKTQDGPKPINPLICSLQCQAALLPSEEWERCQSFSFFTPSTLGLNDKEDLGSKSVGEGPYSELAKLSGSFLKELEKSKFLPSISTKENTLSKSLEEKLRGLSDGFREGAESELMRDAQLNDGAMETGTLYLAEEDPKEQVKRYGGFLRKYPKRSSEVAGEGDGDSMGHEDLYKRYGGFLRRIRPKLKWDNQKRYGGFLRRQFKVVTRSQEDPNAYSGELFDA

SEQ ID NO: 251 - DNA, 12 bp, Artificial Sequence
  Description of Artificial Sequence: Synthetic oligonucleotide
tttctgccctca

SEQ ID NO: 252 - DNA, 36 bp, Artificial Sequence
  Description of Artificial Sequence: Synthetic oligonucleotide
tttctgccctcagaatttggagtagacgtagacaga

SEQ ID NO: 253 - protein, 2087 aa, Artificial Sequence
  Description of Artificial Sequence: Synthetic polypeptide
MVKKIANDVSNKLFPLPKGFGDFVGIEDHIKAIKSILCLESKEARIMVGIWGQSGIGKSTIGRALFSQLSSQFHHRAFITYKSTSGSDVSGMKLSWEKELLSEILGQKDIKIDHFGVVEQRLKHKKVLILLDDVDNLEFLKTLVGKAEWFGSGSRIIVITQDKQLLKAHEIDLVYEVELPSQGLALKMISQYAFGKDSPPDDFKELAFEVAELVGSLPLGLSVLGSSLKGRDKDEWVKMMPRLRNDSDDKIEETLRVGYDRLNKKNRDNVKELLEDDVGLTMLADKSLIRITPDGDIEMHNLLEKLGREIDRAKSKGNPAKRQFLTNFEDIQEVVTEKTGTETVLGIRVPPTVLFSTRPLLVINEESFKGMQIGLWSKIDLPQGLVYLPLKLKLLKWNYCPLKSLPSTFKAEYLVNLIMKYSKLEKLWEGTLPLGSLKKMDLGCSNNLKEIPDLSLAINLEELNLSKCESLVTLPSSIQNAIKLRTLYCSGVLLIDLKSLEGMCNLEYLSVDWSSMEGTQGLIYLPRKLKRLWWDYCPVKRLPSNFKAEYLVELRMENSDLEKLWDGTQPLGSLKEMYLHGSKYLKEIPDLSLAINLERLYLFGCESLVTLPSSIQNATKLINLDMRDCKKLESFPTDLNLESLEYLNLTGCPNLRNFPAIKMGCSYFEILQDRNEIEVEDCFWNKNLPAGLDYLDCLMRCMPCEFRPEYLTFLDVSGCKHEKLWEGIQIHALLDGYELAGHLDGSIETPAPTLTTNNVVSANPQYTLWKRQDRLIFSALIGAISPPVQPLVSRATKASQIWKTLTNTYAKSSYDHIKQLRTQIKQLKKGTKTIDEYVLSHTTLLDQLAILGKPMEHEEQVERILEGLPEDYKTVVDQIEGKDNTPSITEIHERLINHEAKLLSTAALSSSSLPMSANVAQQRHHNNNRNNNQNKNRTQGNTYTNNWQPSANNKSGQRPFKPYLGKCQICNVQGHSARRCPQLQAMQPSSSSSASTFTPWQPRANLAMGAPYTANNWLLDSGATHHITSDLNALALHQPYNGDDVMIADGTSLKITKTGSTFLPSNARDLTLNKVLYVPDIQKNLVSVYRLCNTNQVSVEFFPASFQVKDLNTGTLLLQGRTKDELYEWPVTNPKATALFTTPSPKTTLSSWHSRLGHPSSSILNTLISKFSLPVSVSASNKLACSDCFINKSHKLPFSISSIKSTSPLEYIFSDVWMSPILSPDNYKYYLQKSQVKSTFIAFKALVENRFQAKIRTLYSDNGGEFIALREFLVSNGISHLTSPPHTPEHNGLSERKHRHIVETGLTLLTQASVPREYWPYAFAAAVYLINRMPTPVLSMESPFQKLFGSKPNYERLRVFGCLCFPWLRPYTHNKLEERSRRCVFLGYSTQTAYLCFDVEHKRLYTSRHVVFDEASFPFSNLTSQNSLPTVTFEQSSSPLVTPILSSSSVLPSCLSSPCTVLHQQQPPVTTPNSPHSSQPTTSPAPLSPHRSTTMDFQVPQPTAPNENGPEPEAQSPPIGPLSNPTHEAFIGPLPNPNRNPTNEIEPTPAPHPKPVKPTTTTTTPNRTTVSDASHQPTAPQQNQHNMKTRAKNNIKKPNTKFSLTATLPNRSPSEPTNVTQALKDKKWRFAMSDEFDAQQRNHTWDLVPHESQLLVGCKWVFKLKYLPNGAIDKYKARLVAKGFNQQYGVDYAETFSPVIKSTTIRLVLDVAVKKDWEIKQLDVNNAFLQGTLTEEVYMAQPPGFIDKDRPTHVCRLRKAIYGLKQAPRAWYMELKQHLFNIGFVNSLSDASLFIYWSDKSSIDAVLTSLAERFSIKDPTDLHYFLGIEATRTKQGLHLMQRKYIKDLLAKHNMADAKPVLTPLPTSPKLTLHGGTKLNDASEYRSVVGSLQYLAFTRPDIAYAVNRLSQLMPQPTEDHWQAAKRVLRYLAGTSTHDWAGDSDDYVSTNAYVIYLGKNPISWSSKKQRGVARSSTESEYRAVANAASEVKWLCSLLSKLHIRLPIRPSIFCDNIGATYLCANPVFHSRMKHIAIDYHFVRNMIQSGALRVSHVSTRDQLADALTKPLSRAHFQSARFKIGVRQLPPS

SEQ ID NO: 254 - protein, 575 aa, Artificial Sequence
  Description of Artificial Sequence: Synthetic polypeptide
MSTSSLRRQMKNIVHNYSEAEIKVREATSNDPWGPSSSLMSEIADLTYNVVAFSEIMSMIWKRLNDHGKNWRHVYKAMTLMEYLIKTGSERVSQQCKENMYAVQTLKDFQYVDRDGKDQGVNVREKAKQLVALLRDEDRLREERAHALKTKEKLAQTATASSAAVGSGPPPEAEQAWPQSSGEEELQLQLALAMSKEEADQPPSCGPEDDVQLQLALSLSREEHDKEERIRRGDDLRLQMAIEESKRETGGKEESSLMDLADVFTTPAPPQASDPWGGPASVPTAVPVAAAASDPWGAPAVPPAADPWGGAAPTPASGDPWRPAAPTGPSVDPWGGTPAPAAGEGPTSDPWGSADGGAPVSGPPSSDPWAPAPAFSDPWGGSPAKPSSNGTAVGGFDTEPDEFSDFDRLRTALPTSGSSTGELELLAGEVPARSPGAFDMSGVGGSLAESVGSPPPAATPTPTPPTRKTPESFLGPNAALVDLDSLVSRPGPTPPGAKASNPFLPSGAPATGPSVTNPFQPAPPATLTLNQLRLSPVPPVPGAPPTYISPLGGGPGLPPMMPPGPPAPNTNPFLL

SEQ ID NO: 255 - protein, 900 aa, Artificial Sequence
  Description of Artificial Sequence: Synthetic polypeptide
MEPPLPVGAQPLATVEGMEMKGPLREPCALTLAQRNGQYELIIQLHEKEQHVQDIIPINSHFRCVQEAEETLLIDIASNSGCKIRVQGDWIRERRFEIPDEEHCLKFLSAVLAAQKAQSQLLVPEQKDSSSWYQKLDTKDKPSVFSGLLGFEDNFSSMNLDKKINSQNQPTGIHREPPPPPFSVNKMLPREKEASNKEQPKVTNTMRKLFVPNTQSGQREGLIKHILAKREKEYVNIQTFRFFVGTWNVNGQSPDSGLEPWLNCDPNPPDIYCIGFQELDLSTEAFFYFESVKEQEWSMAVERGLHSKAKYKKVQLVRLVGMMLLIFARKDQCRYIRDIATETVGTGIMGKMGNKGGVAVRFVFHNTTFCIVNSHLAAHVEDFERRNQDYKDICARMSFVVPNQTLPQLNIMKHEVVIWGDLNYRLCMPDANEVKSLINKKDLQRLLKFDQLNIQRTQKKAFVDFNEGEIKFIPTYKYDSKTDRWDSSGKCRVPAWCDRILWRGTNVNQLNYRSHMELKTSDHKPVSALFHIGVKVVDERRYRKVFEDSVRIMDRMENDFLPSLELSRREFVFENVKFRQLQKEKFQISNNGQVPCHFSFIPKLNDSQYCKPWLRAEPFEGYLEPNETVDISLDVYVSKDSVTILNSGEDKIEDILVLHLDRGKDYFLTISGNYLPSCFGTSLEALCRMKRPIREVPVTKLIDLEEDSFLEKEKSLLQMVPLDEGASERPLQVPKEIWLLVDHLFKYACHQEDLFQTPGMQEELQQIIDCLDTSIPETIPGSNHSVAEALLIFLEALPEPVICYELYQRCLDSAYDPRICRQVISQLPRCHRNVFRYLMAFLRELLKFSEYNSVNANMIATLFTSLLLRPPPNLMARQTPSDRQRAIQFLLGFLLGSEED

SEQ ID NO: 256 - protein, 1621 aa, Artificial Sequence
  Description of Artificial Sequence: Synthetic polypeptide
MSESGNTTSMPGCGRMCALRSTWSKRAFLVACKDGALTSDGRCPQYGCGALVSITKGVQQPKKTASAKVVKCLCWVQPARWCEKHSKGPASPNGSVTTKRSNSARAAPAPLPYKKQTCDVVVTVGPLELVYPALVSEELPTPVAATPTKVEEVPIPELPLWLAPAWMVEQPYAATPEVLCLTQREEFALLKKRLTRKGKLLQRRATHARFEARAALARVRAATQRKVEEVTALVIKGRRILAAHQLLRELEEVAPLSQAQEQLVASSCAAAAARQEECASFLRRAKAWRKSISATPPVAATAVASKVVSATMPWAHLGLSLGGLLAVPTLDGTLGAKQWNAKTIATWVLKPVVSCVQSVHAKVRDWLHSQPEVGVTNTKVPLVLPEVCLGVLSPPSLSEEIVDNPQETSQSGIWHPEMGVRNIYVFHDDSWETSPEEDENYTYTFSRQCGIPYLLVEGRGAEERKNTILGWDFSLHNDGFEFLPSPEEGYTKELVTPVALEEEDKYSTASSCGFFSLDDVSSAITIQCPGLLSADADVHFFDGPGYRCSSRPRDFRPPVVRGCDYESRVKASIQRKIENPLQERFITVLREKRKKNKKKEFHSFSACFAFKRKQIQWPPTPNEMVNEWEEYCIAQAWLPFEVVVTDEIEDVTPLYPGGRDYNCNSQLLFPLAPLSTVYCDDSCFHPNDGWTTDGNGKHFRLSPQFVLPDVPIPIVHRVTRQLPQFLYDLGIGDLTCNSGYQAENLQEEIQERMEDRSEEKPVPSLDTLISKLSKRSTKVKGAGENRYADRHSLTEKAIFHQPGALSRMRSGKEKTIVAANHNSDQISVRMAECGKPVFTPLPRMSDEMLRKFLEKGLGSTSTVALDIGIQSHIPQGMPTVAFVNVMDTRIEDPLYSSLCGSYIDLGRDRAKTLCLPLVNFPMSKLAEDVDDVLNGLMLCTHFQDSTKFGVGKPAFQYGTLEFQEFKPSAYSDFSRVRDNWDAIAKQQNTPNDRILAGFSVLGAVSQAYNQALPVFKSVELVAPPKRKPVVATFQNPTTLGRSNTTRSFRMPTMDLPRSTGRDAPIPIVHRRNNNDVHFDEATPARFSTCDSGLVADTTLAFAKMYQCKKDAKAGHVLATIDIQECVFEDNRRVALDWLAHGLASFKYDLQLTVDSNPFVGVTLGITVDAFDRLLPQISDEVIAVPLAFQLPTYLFPISKKGTFTQTIDFAAIAGYNFFPHVAAFGRPKIIVYIVSDNDLPASDTWMCLVELHMTRLESSTLACSPTLVLPQAFGGDLPLDLWRGPYTFPLGGGTKRLSTSLDIGTSTTTVSGWRTVSPAAYALFLQGHGGSLVGEVVHTGSAAVSCALHLCISFGGAPPTLEEALVFPGFRLPSGEGKFHIKVQTPYGRLSTLTPDCALYVYLAGGPIAVAPMSVPYQFCIHLERLVDDGAPPRTIGLIREFNWATINNFKSDDITFAIPARLSDLVLTCGDVTMSTNPLALLIGSCGFFRGNLTVVLEWATFLKAGDKEGTVQLTTCRGMINNVKGVRNAIQKKVVNLSLVGSVSRYLNVGDFTGFAQSGGQVGYDEIFLEFSTNKAKQIRYLNINVELDENFELYGRTIIPLKNTAPAFASTSSAPNES

SEQ ID NO: 257 - protein, 5 aa, Artificial Sequence
  Description of Artificial Sequence: Synthetic peptide
KFLPS

SEQ ID NO: 258 - protein, 5 aa, Artificial Sequence
  Description of Artificial Sequence: Synthetic peptide
FLPSI

SEQ ID NO: 259 - protein, 5 aa, Artificial Sequence
  Description of Artificial Sequence: Synthetic peptide
RFLPS

SEQ ID NO: 260 - protein, 5 aa, Artificial Sequence
  Description of Artificial Sequence: Synthetic peptide
FLPSE

SEQ ID NO: 261 - protein, 4 aa, Artificial Sequence
  Description of Artificial Sequence: Synthetic peptide
SFLK

SEQ ID NO: 262 - protein, 4 aa, Artificial Sequence
  Description of Artificial Sequence: Synthetic peptide
WERC

SEQ ID NO: 263 - protein, 6 aa, Artificial Sequence
  Description of Artificial Sequence: Synthetic peptide
FSFFTP

SEQ ID NO: 264 - protein, 4 aa, Artificial Sequence
  Description of Artificial Sequence: Synthetic peptide
FFNP

SEQ ID NO: 265 - protein, 4 aa, Artificial Sequence
  Description of Artificial Sequence: Synthetic peptide
FPST

SEQ ID NO: 266 - protein, 4 aa, Artificial Sequence
  Description of Artificial Sequence: Synthetic peptide
SFLG

SEQ ID NO: 267 - protein, 4 aa, Artificial Sequence
  Description of Artificial Sequence: Synthetic peptide
YSEL

SEQ ID NO: 268 - protein, 4 aa, Artificial Sequence
  Description of Artificial Sequence: Synthetic peptide
SYLG

SEQ ID NO: 269 - protein, 4 aa, Artificial Sequence
  Description of Artificial Sequence: Synthetic peptide
SWLG

SEQ ID NO: 270 - protein, 5 aa, Artificial Sequence
  Description of Artificial Sequence: Synthetic peptide
FFTPS

SEQ ID NO: 271 - protein, 9 aa, Artificial Sequence
  Description of Artificial Sequence: Synthetic peptide
SFLSFFTPS

SEQ ID NO: 272 - protein, 5 aa, Artificial Sequence
  Description of Artificial Sequence: Synthetic peptide
NPFQP

SEQ ID NO: 273 - protein, 5 aa, Artificial Sequence
  Description of Artificial Sequence: Synthetic peptide
MSFLK

SEQ ID NO: 274 - protein, 6 aa, Artificial Sequence
  Description of Artificial Sequence: Synthetic peptide
FLPPPS

SEQ ID NO: 275 - protein, 5 aa, Artificial Sequence
  Description of Artificial Sequence: Synthetic peptide
FLPPS

SEQ ID NO: 276 - protein, 5 aa, Artificial Sequence
  Description of Artificial Sequence: Synthetic peptide
MFPST

SEQ ID NO: 277 - protein, 9 aa, Artificial Sequence
  Description of Artificial Sequence: Synthetic peptide
FLPPPPPPS

SEQ ID NO: 278 - protein, 8 aa, Artificial Sequence
  Description of Artificial Sequence: Synthetic peptide
FLPPPPPS

SEQ ID NO: 279 - protein, 6 aa, Artificial Sequence
  Description of Artificial Sequence: Synthetic peptide
FGGFIM

SEQ ID NO: 280 - protein, 4 aa, Artificial Sequence
  Description of Artificial Sequence: Synthetic peptide
KLFS

SEQ ID NO: 281 - protein, 4 aa, Artificial Sequence
  Description of Artificial Sequence: Synthetic peptide
CREW

SEQ ID NO: 282 - protein, 6 aa, Artificial Sequence
  Description of Artificial Sequence: Synthetic peptide
PTFFSF

SEQ ID NO: 283 - protein, 4 aa, Artificial Sequence
  Description of Artificial Sequence: Synthetic peptide
PNFF

SEQ ID NO: 284 - protein, 4 aa, Artificial Sequence
  Description of Artificial Sequence: Synthetic peptide
TSPF

SEQ ID NO: 285 - protein, 4 aa, Artificial Sequence
  Description of Artificial Sequence: Synthetic peptide
GLFS

SEQ ID NO: 286 - protein, 4 aa, Artificial Sequence
  Description of Artificial Sequence: Synthetic peptide
LESY

SEQ ID NO: 287 - protein, 4 aa, Artificial Sequence
  Description of Artificial Sequence: Synthetic peptide
GLYS

SEQ ID NO: 288 - protein, 4 aa, Artificial Sequence
  Description of Artificial Sequence: Synthetic peptide
GLWS

SEQ ID NO: 289 - protein, 5 aa, Artificial Sequence
  Description of Artificial Sequence: Synthetic peptide
SPTFF

SEQ ID NO: 290 - protein, 9 aa, Artificial Sequence
  Description of Artificial Sequence: Synthetic peptide
SPTFFSLFS

SEQ ID NO: 291 - protein, 5 aa, Artificial Sequence
  Description of Artificial Sequence: Synthetic peptide
PQFPN

SEQ ID NO: 292 - protein, 5 aa, Artificial Sequence
  Description of Artificial Sequence: Synthetic peptide
KLFSM

SEQ ID NO: 293 - protein, 7 aa, Artificial Sequence
  Description of Artificial Sequence: Synthetic peptide
SPPPPLF

SEQ ID NO: 294 - protein, 5 aa, Artificial Sequence
  Description of Artificial Sequence: Synthetic peptide
SPPLF

SEQ ID NO: 295 - protein, 5 aa, Artificial Sequence
  Description of Artificial Sequence: Synthetic peptide
TSPFM

SEQ ID NO: 296 - protein, 10 aa, Artificial Sequence
  Description of Artificial Sequence: Synthetic peptide
SPPPPPPPLF

SEQ ID NO: 297 - protein, 9 aa, Artificial Sequence
  Description of Artificial Sequence: Synthetic peptide
SPPPPPPLF

SEQ ID NO: 298 - protein, 6 aa, Artificial Sequence
  Description of Artificial Sequence: Synthetic peptide
MIFGGF

SEQ ID NO: 299 - protein, 174 aa, Artificial Sequence
  Description of Artificial Sequence: Synthetic polypeptide
MAWQGLVLAACLLMFPSTTADCLSRCSLCAVKTQDGPKPINPLICSLQCQAALLPSEEWERCQSFLSFFTPSTLGLNDKEDLGSKSVGEGPYSELAKLSGSFLKELEKSKFLPSISTKENTLSKSLEEKLRGLSDGFREGAESELMRDAQLNDGAMETGTLYLAEEDPKEQVKR

SEQ ID NO: 300 - protein, 100 aa, Artificial Sequence
  Description of Artificial Sequence: Synthetic polypeptide
MARFLTLCTWLLLLGPGLLATVRAECSQDCATCSYRLVRPADINFLACVMECEGKLPSLKIWETCKELLQLSKPELPQDGTSTLRENSKPEESHLLAKRY

SEQ ID NO: 301 - protein, 267 aa, Artificial Sequence
  Description of Artificial Sequence: Synthetic polypeptide
MARFLTLCTWLLLLGPGLLATVRAECSQDCATCSYRLVRPADINFLACVMECEGKLPSLKIWETCKELLQLSKPELPQDGTSTLRENSKPEESHLLAKRYGGFMKRYGGFMKKMDELYPMEPEEEANGSEILAKRYGGFMKKDAEEDDSLANSSDLLKELLETGDNRERSHHQDGSDNEEEVSKRYGGFMRGLKRSPQLEDEAKELQKRYGGFMRRVGRPEWWMDYQKRYGGFLKRFAEALPSDEEGESYSKEVPEMEKRYGGFMRF

SEQ ID NO: 302 - protein, 267 aa, Artificial Sequence
  Description of Artificial Sequence: Synthetic polypeptide
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 303 - protein, 236 aa, Artificial Sequence
  Description of Artificial Sequence: Synthetic polypeptide
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKR

SEQ ID NO: 304 - protein, 7 aa, Artificial Sequence
  Description of Artificial Sequence: Synthetic peptide
FLPSPLF

SEQ ID NO: 305 - protein, 8 aa, Artificial Sequence
  Description of Artificial Sequence: Synthetic peptide
FLPSSPLF

SEQ ID NO: 306 - protein, 4 aa, Artificial Sequence
  Description of Artificial Sequence: Synthetic peptide
PFLP

SEQ ID NO: 307 - protein, 4 aa, Artificial Sequence
  Description of Artificial Sequence: Synthetic peptide
SLFP

SEQ ID NO: 308 - protein, 4 aa, Artificial Sequence
  Description of Artificial Sequence: Synthetic peptide
FPSA

SEQ ID NO: 309 - skipped

SEQ ID NO: 310 - protein, 4 aa, Artificial Sequence
  Description of Artificial Sequence: Synthetic peptide
AFLP

SEQ ID NO: 311 - protein, 4 aa, Artificial Sequence
  Description of Artificial Sequence: Synthetic peptide
TLPF

SEQ ID NO: 312 - protein, 4 aa, Artificial Sequence
  Description of Artificial Sequence: Synthetic peptide
PPLF

SEQ ID NO: 313 - protein, 4 aa, Artificial Sequence
  Description of Artificial Sequence: Synthetic peptide
FPSP

SEQ ID NO: 314 - protein, 4 aa, Artificial Sequence
  Description of Artificial Sequence: Synthetic peptide
FLVS

SEQ ID NO: 315 - protein, 4 aa, Artificial Sequence
  Description of Artificial Sequence: Synthetic peptide
PLFP

SEQ ID NO: 316 - protein, 4 aa, Artificial Sequence
  Description of Artificial Sequence: Synthetic peptide
FLFS

SEQ ID NO: 317 - protein, 4 aa, Artificial Sequence
  Description of Artificial Sequence: Synthetic peptide
LFSF

SEQ ID NO: 318 - protein, 4 aa, Artificial Sequence
  Description of Artificial Sequence: Synthetic peptide
MFTS

SEQ ID NO: 319 - protein, 4 aa, Artificial Sequence
  Description of Artificial Sequence: Synthetic peptide
PSLF

SEQ ID NO: 320 - protein, 4 aa, Artificial Sequence
  Description of Artificial Sequence: Synthetic peptide
PSSF

SEQ ID NO: 321 - protein, 4 aa, Artificial Sequence
  Description of Artificial Sequence: Synthetic peptide
FTPS

SEQ ID NO: 322 - protein, 4 aa, Artificial Sequence
  Description of Artificial Sequence: Synthetic peptide
FLSF

SEQ ID NO: 323 - protein, 4 aa, Artificial Sequence
  Description of Artificial Sequence: Synthetic peptide
LPSF

SEQ ID NO: 324 - protein, 4 aa, Artificial Sequence
  Description of Artificial Sequence: Synthetic peptide
FKPS

SEQ ID NO: 325 - protein, 4 aa, Artificial Sequence
  Description of Artificial Sequence: Synthetic peptide
FLSP

SEQ ID NO: 326 - protein, 4 aa, Artificial Sequence
  Description of Artificial Sequence: Synthetic peptide
FPLS

SEQ ID NO: 327 - protein, 4 aa, Artificial Sequence
  Description of Artificial Sequence: Synthetic peptide
FPSL

SEQ ID NO: 328 - protein, 4 aa, Artificial Sequence
  Description of Artificial Sequence: Synthetic peptide
FSPL

SEQ ID NO: 329 - protein, 4 aa, Artificial Sequence
  Description of Artificial Sequence: Synthetic peptide
FSLP

SEQ ID NO: 330 - protein, 4 aa, Artificial Sequence
  Description of Artificial Sequence: Synthetic peptide
LFPS

SEQ ID NO: 331 - protein, 4 aa, Artificial Sequence
  Description of Artificial Sequence: Synthetic peptide
LFSP

SEQ ID NO: 332 - protein, 4 aa, Artificial Sequence
  Description of Artificial Sequence: Synthetic peptide
LPFS

SEQ ID NO: 333 - protein, 4 aa, Artificial Sequence
  Description of Artificial Sequence: Synthetic peptide
LSPF

SEQ ID NO: 334 - protein, 4 aa, Artificial Sequence
  Description of Artificial Sequence: Synthetic peptide
LSFP

SEQ ID NO: 335 - protein, 4 aa, Artificial Sequence
  Description of Artificial Sequence: Synthetic peptide
PFLS

SEQ ID NO: 336 - protein, 4 aa, Artificial Sequence
  Description of Artificial Sequence: Synthetic peptide
PFSL

SEQ ID NO: 337 - protein, 4 aa, Artificial Sequence
  Description of Artificial Sequence: Synthetic peptide
PLSF

SEQ ID NO: 338 - protein, 4 aa, Artificial Sequence
  Description of Artificial Sequence: Synthetic peptide
PLFS

SEQ ID NO: 339 - protein, 4 aa, Artificial Sequence
  Description of Artificial Sequence: Synthetic peptide
PSFL

SEQ ID NO: 340 - protein, 4 aa, Artificial Sequence
  Description of Artificial Sequence: Synthetic peptide
SFLP

SEQ ID NO: 341 - protein, 4 aa, Artificial Sequence
  Description of Artificial Sequence: Synthetic peptide
SFPL

SEQ ID NO: 342 - protein, 4 aa, Artificial Sequence
  Description of Artificial Sequence: Synthetic peptide
SPFL

SEQ ID NO: 343 - protein, 4 aa, Artificial Sequence
  Description of Artificial Sequence: Synthetic peptide
SLPF

SEQ ID NO: 344 - protein, 3 aa, Artificial Sequence
  Description of Artificial Sequence: Synthetic peptide
FLP

SEQ ID NO: 345 - protein, 3 aa, Artificial Sequence
  Description of Artificial Sequence: Synthetic peptide
FPL

SEQ ID NO: 346 - protein, 3 aa, Artificial Sequence
  Description of Artificial Sequence: Synthetic peptide
PLF

SEQ ID NO: 347 - protein, 3 aa, Artificial Sequence
  Description of Artificial Sequence: Synthetic peptide
LPF

SEQ ID NO: 348 - protein, 3 aa, Artificial Sequence
  Description of Artificial Sequence: Synthetic peptide
LFP

SEQ ID NO: 349 - protein, 3 aa, Artificial Sequence
  Description of Artificial Sequence: Synthetic peptide
PFL

SEQ ID NO: 350 - protein, 10 aa, Artificial Sequence
  Description of Artificial Sequence: Synthetic peptide
FLPSDFFPSV

SEQ ID NO: 351 - protein, 23 aa, Artificial Sequence
  Description of Artificial Sequence: Synthetic peptide
  C-terminus amidated
TPPAYRPPNAPILFLPSDFFPSV

SEQ ID NO: 352 - protein, 23 aa, Artificial Sequence
  Description of Artificial Sequence: Synthetic peptide
TPPAYRPPNAPILFLPSDFFPSV

SEQ ID NO: 353 - protein, 26 aa, Artificial Sequence
  Description of Artificial Sequence: Synthetic peptide
  C-terminus amidated
TPPAYRPPNAPILAAAFLPSDFFPSV

SEQ ID NO: 354 - protein, 26 aa, Artificial Sequence
  Description of Artificial Sequence: Synthetic peptide
TPPAYRPPNAPILAAAFLPSDFFPSV

SEQ ID NO: 355 - protein, 27 aa, Artificial Sequence
  Description of Artificial Sequence: Synthetic peptide
  N-terminus acetylated
QYIKANSKFIGITEAAAFLPSDFFPSV

SEQ ID NO: 356 - protein, 27 aa, Artificial Sequence
  Description of Artificial Sequence: Synthetic peptide
QYIKANSKFIGITEAAAFLPSDFFPSV

SEQ ID NO: 357 - protein, 11 aa, Artificial Sequence
  Description of Artificial Sequence: Synthetic peptide
KKFLPSEFGHD

SEQ ID NO: 358 - protein, 12 aa, Artificial Sequence
  Description of Artificial Sequence: Synthetic peptide
FLPSEFGHDVDR

SEQ ID NO: 359 - protein, 17 aa, Artificial Sequence
  Description of Artificial Sequence: Synthetic peptide
ATVELLSFLPSDFFPSV

SEQ ID NO: 360 - protein, 16 aa, Artificial Sequence
  Description of Artificial Sequence: Synthetic peptide
TVELLSFLPSDFFPSV

SEQ ID NO: 361 - protein, 15 aa, Artificial Sequence
  Description of Artificial Sequence: Synthetic peptide
VELLSFLPSDFFPSV

SEQ ID NO: 362 - protein, 14 aa, Artificial Sequence
  Description of Artificial Sequence: Synthetic peptide
ELLSFLPSDFFPSV

SEQ ID NO: 363 - protein, 13 aa, Artificial Sequence
  Description of Artificial Sequence: Synthetic peptide
LLSFLPSDFFPSV

SEQ ID NO: 364 - protein, 12 aa, Artificial Sequence
  Description of Artificial Sequence: Synthetic peptide
LSFLPSDFFPSV

SEQ ID NO: 365 - protein, 10 aa, Artificial Sequence
  Description of Artificial Sequence: Synthetic peptide
FLPSDFFPSV

SEQ ID NO: 366 - protein, 9 aa, Artificial Sequence
  Description of Artificial Sequence: Synthetic peptide
FLPSDFFPS

SEQ ID NO: 367 - protein, 9 aa, Artificial Sequence
  Description of Artificial Sequence: Synthetic peptide
SFLPSDFFP

SEQ ID NO: 368 - protein, 9 aa, Artificial Sequence
  Description of Artificial Sequence: Synthetic peptide
LSFLPSDFF

SEQ ID NO: 369 - protein, 9 aa, Artificial Sequence
  Description of Artificial Sequence: Synthetic peptide
LLSFLPSDF

SEQ ID NO: 370 - protein, 9 aa, Artificial Sequence
  Description of Artificial Sequence: Synthetic peptide
ELLSFLPSD

SEQ ID NO: 371 - protein, 9 aa, Artificial Sequence
  Description of Artificial Sequence: Synthetic peptide
VELLSFLPS

SEQ ID NO: 372 - protein, 4 aa, Artificial Sequence
  Description of Artificial Sequence: Synthetic peptide
YLPS

SEQ ID NO: 373 - protein, 4 aa, Artificial Sequence
  Description of Artificial Sequence: Synthetic peptide
WLPS

SEQ ID NO: 374 - protein, 4 aa, Artificial Sequence
  Description of Artificial Sequence: Synthetic peptide
SPLY

SEQ ID NO: 375 - protein, 4 aa, Artificial Sequence
  Description of Artificial Sequence: Synthetic peptide
SPLW

(19) United States
(12) Patent Application Publication
(10) Pub. No.: US 2009/0069237 A1
(43) Pub. Date: Mar. 12, 2009

(54) METHODS AND COMPOSITIONS FOR TREATING CONDITIONS

(76) Inventor: Hanna Skubatch, Seattle, WA (US)

Correspondence Address: WILSON SONSINI GOODRICH & ROSATI, 650 PAGE MILL ROAD, PALO ALTO, CA, 94304-1050, US

(21) Appl. No.: 12/176,235

(22) Filed: Jul. 18, 2008

Related U.S. Application Data
(60) Provisional application No. 60/950,569, filed on Jul. 18, 2007.

(51) Int. Cl. A61K 38/16; C07K 14/00; A61P 25/00

(52) U.S. Cl. 514/12; 530/350

(57) ABSTRACT

The invention relates to compositions that can be used to treat, prevent, and modulate pain, inflammation, and metabolic processes in various organisms including plants and animals. Such compositions can be formulated with an acceptable pharmaceutical excipient for administration to a human or a plant. The compositions can be administered topically or for systemic use.

### CROSS-REFERENCE

This claims the benefit of U.S. Provisional Application No. 60/950,569, filed on Jul. 18, 2007, which is herein incorporated by reference in its entirety.

### BACKGROUND

There are many conditions that affect plants and animals, including but not limited to pain, metabolic conditions/thermoregulation conditions (e.g., fever), pathogen infections, and neurological/neurodegenerative conditions (e.g., Alzheimers disease). There is a significant need to identify new compositions and methods to treat and/or prevent such conditions.

### INCORPORATION BY REFERENCE

All publications and patent applications mentioned in this specification are herein incorporated by reference to the same extent as if each individual publication or patent application was specifically and individually indicated to be incorporated by reference.

### SUMMARY OF THE INVENTION

The invention herein involves compositions comprising, consisting essentially of, or consisting of a polypeptide of the invention or a homolog, analog, mimetic, salt, prodrug, metabolite, or fragment thereof or combination. In some embodiments, a polypeptide comprises, consists essentially of, or consists of one or more amino acid sequences selected from the group consisting of SEQ ID NOs: 1-349 or an amino acid sequence selected from the group consisting of SEQ ID NOs 1-244, 248-249, 257-349, or a reverse sequence of any of the above or of SEQ ID NOs: 1-244, 248-249, 257-279, or 306-349.

For example, in some embodiments, a composition comprises a polypeptide having amino acid sequence of any one of SEQ ID NO: 1-349 or of any one of SEQ ID NO: 1-14 or 50-244, 248-249, 257-349, or of any one of SEQ ID NO: 1, 2, 153, 304-349 or of any one of SEQ ID NO: 1, 153, 304 or 305. In some embodiments, a composition comprises a polypeptide having an amino acid sequence which is the reverse of any one of SEQ ID NO: 1-349 or any one of SEQ ID NO: 1, 153, 304-349. The invention herein also contemplates homologs, analogs (especially small molecule analogs), mimetics, salts, prodrugs, metabolites, and fragments of the above polypeptides and compositions comprising the same.

In one aspect, an isolated polypeptide consisting essentially of FLPS or an analog thereof, but specifically excluding all of SEQ ID NOS: 350-371 is provided.

In another aspect, a fragment of any one of SEQ ID NOS: 245-247 and 250, 253-256 wherein said fragment comprises FLPS or an analog thereof is provided.

In another aspect, an isolated polypeptide comprising FLPS having less than 100 or 50 or 30 amino acid residues or an analog thereof, but specifically excluding all of SEQ ID NOS: 350-371, is provided.

In another aspect, an isolated polypeptide consisting of FLPS and 0, 1, 2, 3, 4, 5, 6, 7, 8, 9, or 10 additional amino acids, but specifically excluding all of SEQ ID NOS: 350-371, is provided.

In another aspect, a method of treating pain by administering to an animal in need thereof a composition including an isolated polypeptide consisting essentially of FLPS or an analog thereof, but specifically excluding all of SEQ ID NOS: 350-371 is provided.

A method of treating pain by administering to an animal in need thereof a composition including a fragment of any one of SEQ ID NOS: 245-247 and 250, 253-256 wherein said fragment comprises FLPS or an analog thereof is provided.

A method of treating pain by administering to an animal in need thereof a composition including an isolated polypeptide comprising FLPS having less than 100 or 50 or 30 amino acid residues or an analog thereof, but specifically excluding all of SEQ ID NOS: 350-371, is provided.

A method of treating pain by administering to an animal in need thereof a composition including an isolated polypeptide consisting of FLPS and 0, 1, 2, 3, 4, 5, 6, 7, 8, 9, or 10 additional amino acids, but specifically excluding all of SEQ ID NOS: 350-371, is provided.

The compositions herein can be used to modulate, prevent, or treat pain, inflammation, infections (e.g., bacterial fungi, viruses, etc.), and metabolic processes or conditions in an organism (plant or animal). As such the compositions herein exhibit both analgesic and anesthetic properties.

Examples of metabolic conditions include, but are not limited to, pain, wound healing, fever, neurological and neurodegenerative conditions, heat production, inflammation, heat production, fever, homeothermy, breakdown of triglycerides, glycolysis, Krebs cycle, fermentation, photosynthesis, metabolic rate, biotic and abiotic stress, secretions, oxidative stress, stress, neoplastic growth, skin condition, cardiovascular conditions, neurological and neurodegenerative conditions, mental and behavioral disorders. Such processes or conditions can occur in a cell, group of cells, or an entire organism.

The compositions herein can be used for modulating, preventing, treating condition(s) in organisms. Such organisms can be animals and/or plants.

In some embodiments, the compositions herein (e.g., a composition comprising a polypeptide of any one or more of SEQ ID NOs: 1-349 or of any one or more of SEQ ID NO: 1-244, 248-249, 257-349, or of any one or more of SEQ ID NO: 1, 153, 304-349 are used to modulate or treat pain, such as nociceptive (non-chronic) pain, neuropathic (chronic) pain, idiopathic pain, headaches, low back pain, cancer pain, arthritis pain, sprains, bone fractures, pain resulting from burns, pain associated with bumps, pain associated with bruises, inflammatory pain (e.g., from an infection or arthritic disorder), pain from obstructions, myofascial pain, pain from nerve trauma (e.g., dystrophy/causalgia), phantom limb pain, entrapment neuropathy (e.g., carpal tunnel syndrome), and peripheral neuropathy.

Thus, in some cases a composition herein (e.g., comprising any one or more of SEQ ID Nos: 1-349 or SEQ ID NO: 1, 153, 304-349 or an analog, salt, metabolite, or prodrug thereof) is administered to an animal to treat pain. Such pain can be non-chronic pain, neuropathic pain, or idiopathic pain. It is further contemplated a compositions comprising a polypeptide described herein (e.g., SEQ ID NO: 1, 153, 304-349 is co-administered with one or more other pain relief medications. For example, a polypeptide described herein, such as SEQ ID NO: 1, 153, or 304-349 can be administered simultaneously with, co-formulated with, or administered in the same therapy as a pain reliever selected from the group consisting of small molecules (e.g., non-narcotic and narcotic analgesics) and peptide opioids.

In some embodiments, the compositions herein (e.g., a composition comprising a polypeptide comprising, consisting essentially, or consisting of any one or more of SEQ ID NOs: 1-349 or 1-244, 248-249, and 257-349, or SEQ ID NO: 1 or 153 or 304 or 305) are used to module or treat inflammatory conditions that may or may not cause pain. Such conditions may show one or more of the following symptoms: redness, heat, tenderness and swelling. Examples of such conditions include, but are not limited to, chronic inflammatory diseases, such as rheumatoid arthritis, inflammatory bowel disease, systemic lupus erythematosus, multiple sclerosis, and type I and II diabetes, asthma, and inflammatory diseases of the central nervous system such as multiple sclerosis, abscess, meningitis, encephalitis and vasculitis.

In some embodiments, the compositions herein (e.g., a composition comprising a polypeptide comprising, consisting essentially, or consisting of any one or more of SEQ ID NOs: 1-349 or any one or more of SEQ ID NO: 1-244, 248-249, 257-349 or any one or more of SEQ ID NO: 1 or 153 or 304 or 305) are used to modulate or treat cardiovascular conditions. Examples of cardiovascular conditions associated with pain and/or inflammation include, but are not limited to, angina, arrhythmia, high blood pressure, stroke, congestive heart failure, atherosclerosis, peripheral artery diseases, high cholesterol levels, and heart attacks.

In some embodiments, the compositions herein (e.g., a composition comprising a polypeptide comprising, consisting essentially, or consisting of any one or more of SEQ ID NOs: 1-349 or any one or more of SEQ ID Nos: 1-244, 248-249, 257-349, or any one or more of SEQ ID NO: 1 or 153 or 304 or 305) are used to modulate or treat fever or abnormal body temperature, whether or not such temperature is associated with pain. Examples of such conditions include infections.

In some embodiments, the compositions herein (e.g., a composition comprising a polypeptide comprising, consisting essentially, or consisting of any one or more of SEQ ID NOs: 1-349 or any one or more of SEQ ID NOS: 1-244, 248-249, 257-349, or any one or more of SEQ ID NO: 1, 153 or 304 or 305) are used to modulate or treat a neurological or neurodegenerative condition or a mental or behavioral disorder. Examples of neurological conditions associated with pain and/or inflammation include, but are not limited to, Alzheimer's disease, amnesia, Aicardi syndrome, amyotrophic lateral sclerosis (Lou Gehrig's disease), anencephaly, anxiety, aphasia, arachnoiditis, Arnold Chiari malformation, attention deficit syndrome, autism, Batten disease, Bell's Palsy, bipolar syndrome, brachial plexus injury, brain injury, brain tumors, childhood depression, Charcot-Marie tooth disease, depression, dystonia, dyslexia, encephalitis, epilepsy, essential tremor, Guillian-Barre syndrome, hydrocephalus, hyperhidrosis, Krabbes disease, learning disabilities, leukodystrophy, meningitis, Moebius syndrome, multiple sclerosis, muscular dystrophy, Parkinson's disease, peripheral neuropathy, obsessive compulsive disorder, postural orthostatic tachycardia syndrome, progressive supranuclear palsy, prosopagnosia, schizophrenia, shingles, Shy-Drager syndrome, spasmodic torticollis, spina bifida, spinal muscular atrophy, stiff man syndrome, synesthesia, syringomyelia, thoracic outlet syndrome, tourette syndrome, toxoplasmosis, and trigeminal neurolagia.

Examples of mental and behavioral disorders include, but are not limited to, anxiety disorder, panic disorder, obsessive-compulsive disorder, post-traumatic stress disorder, social phobia (or social anxiety disorder), specific phobias, and generalized anxiety disorder. Any of the above conditions can also be accompanied by or manifested by other conditions such as depression, drug abuse, or alcoholism.

In some embodiments, the compositions herein are used to treat fever that occurs with many different conditions such as inflammation and infectious diseases.

In some embodiments, the compositions herein are used to modulate or treat neoplastic growth. Examples of neoplastic growth include, but are not limited to, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, reticulum cell sarcoma, myeloma, giant cell tumor, small-cell lung tumor, gallstones, islet cell tumor, primary brain tumor, acute and chronic lymphocytic and granulocytic tumors, hairy-cell leukemia, adenoma, hyperplasia, medullary carcinoma, pheochromocytoma, mucosal neurons, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforme, leukemias, lymphomas, malignant melanomas, epidermoid carcinomas, and other carcinomas and sarcomas.

Thus, in some embodiments, a composition herein (e.g., a composition comprising any one or more of SEQ ID NO: 1-349 1 or any one or more of SEQ ID NOS: 153 or 304 or 305) can be administered simultaneously with, co-formulated with, or administered in the same therapy as an anti-neoplastic agent.

In some embodiments, the compositions herein are used to modulate and treat abnormal temperature associated with non-rapid eye movement (NREM) during sleep, thermotaxis of human spermatozoa toward fertilization site (isthmic-ampullary junction) at ovulation, and hot flashes in postmenopausal women.

The compositions herein can be used to treat addition in an animal by administering to the animal any of the compositions described herein (e.g., peptides, small molecules, nucleic acids encoding the above, etc.).

In some embodiments, the compositions herein are used to treat or prevent plants/crops from yield losses. Examples of plants that may be treated with the compositions herein include major crops (corn, soybeans, hay, wheat, cotton, sorghum, rice, etc.) Examples of conditions resulting in crop losses are diseases caused by bacteria, viruses, and fungi. Other examples of conditions that may result in crop losses that can be preventable or diminished by the compositions herein include stress conditions such as drought, freezing, oxidative stress, unfavorable or reduced temperatures, infection by pathogens and other unfavorable environmental conditions.

In some embodiments, the compositions herein are used to modulate (e.g., increase, decrease or control) temperature. Examples of plants that may be treated with the compositions herein include ornamental crops: flower bulbs (e.g., Tulips, Daffodils, Hyacinths, Crocus, Dutch iris, Allium etc.), cut flowers (e.g., roses, carnation, lily, gladiolus, bird of paradise, etc); vegetable crop (e.g., tomato, cucumber, celery, eggplants, pumpkins, carrot, lettuce, zucchini, etc.); fruit crops (e.g., apple, citrus, peach, pear, plums, banana, pineapple, olive, avocado, papaya, mango, nuts, berries, and other types of agricultural crops such as grain (e.g., corn, soybeans, hay, wheat, barley, corn, cotton, sorghum, and rice) and trees used for lumber (e.g, Douglas fir, cedar, maple, oak, poplar).

In some embodiments, the compositions herein are used to modulate (e.g., increase, decrease or control) seed production. Examples of plants that may be treated with the compositions herein include seeds of ornamental crops, vegetable crops, fruit and nut crops, seeds of other types of agricultural crops, or other plants disclosed herein

In some embodiments, the compositions herein are used to modulate (e.g., increase, decrease or control) secretory products in plants or animals. Such secretary products include, but are not limited to, small volatiles and non-volatile compounds such as terpenes, fatty acid oxidative products, and amines, as well as high molecular weight molecules such as polypeptides and polysaccharides. Such secretions can be, for example, involved in inter-, intra-cellular communications and/or diseases.

The invention herein also provides for nucleic acids that encode the compositions herein and nucleic acid that are complementary to nucleic acids that encode the compositions herein. Nucleic acids that encode the compositions herein can be inserted into a vector to express the polypeptides herein recombinantly. Nucleic acids that are complementary to the polypeptides herein can be used to modulate the expression of certain polypeptides and as diagnostics or research tools.

The compositions herein can be formulated with one or more carriers or excipients for delivery to an organism such as an animal or a plant. Such carriers can be, for example, pharmaceutical carriers, veterinary carriers, and agricultural carriers. For delivery to an animal, the compositions herein may be administered in a therapeutically effective dose to reduce, inhibit, eliminate, ameliorate or prevent a condition. Similarly, for delivery to a plant (e.g., a crop plant), the compositions herein can be delivered in an effective dose to reduce, inhibit, eliminate, ameliorate or prevent a condition.

The invention also provides for antibodies or antibody fragments that are specific to the polypeptides herein. Such antibodies or antibody fragments can be used therapeutically, prophylactically, or for research purposes. Such antibodies or antibody fragments are preferably humanized and/or monoclonal.

The invention herein also provides for methods for screening for binding molecules (receptors) and for agents (ligand) that modulate the composition herein, or their binding to receptors. Binding affinity is determined by a competitive assay using labeled agents (e.g., biotinylated or fluorescent) incubated with the receptors in the presence of various concentrations of a composition of the invention. The affinity binding constant, Kₐ, has to be of greater than or equal to about 10⁵ to 10⁷M⁻¹, or greater than or equal to about 10⁸M⁻¹, or greater than or equal to about 10⁹M⁻¹ or greater than or equal to about 10¹⁰M⁻¹. In certain embodiments binding affinity constants of peptides for the binding polypeptides may exceed 10¹¹ to 10¹²M⁻¹. Affinities of binding polypeptides for ligands according to the present invention can be readily determined using conventional techniques, for example those described by Scatchard et al. (1949 Ann. N.Y. Acad. Sci. 51:660), or by other various techniques described in the scientific literature.

The invention herein also provides small molecules and/or peptidomimetics of the polypeptides herein and methods for making the same.

In some aspects the invention herein contemplates methods of treating addiction in an animal by administering to the animal a composition comprising any of the compositions herein.

The present invention also contemplates a method for treating a plant or an animal suffering from a condition comprising administering to said plant or animal a composition comprising a peptide selected from the group consisting of SEQ ID NO: 306-349 or a small molecule thereof. In some cases the condition described above can be pain, a neurodegenerative or neurological condition, an addiction, Alzheimer's disease, a pathogen infection, a metabolic disorder, fever, inflammation, or neoplastic growth.

The present invention also relates to compositions comprising a peptide comprising, consisting essentially of, or consisting of SEQ ID NO: 306-349 or a small molecule thereof as well as pharmaceutical excipients comprising the above composition and a pharmaceutical excipient or an agricultural formulation comprising the composition above an agricultural excipient or a cosmetic formulation comprising the composition above with a cosmetic excipient, etc.

In some cases, the composition (or formulation derived thereof) comprises, consists essentially of, or consists of a peptide comprising Phe-Leu, Leu-Phe, Pro-Ser, or Ser-Pro or a small molecule thereof.

### BRIEF DESCRIPTION OF THE DRAWINGS

FIG. 1 illustrates the efficacy of SEQ ID NO: 1 in relieving pain in rats 3 days after surgery.

FIG. 2 illustrates the efficacy of SEQ ID NO: 1 in relieving pain in rats 3 h after surgery

FIG. 3 illustrates the efficacy of SEQ ID NO: 1 in relieving pain in rats after surgery

FIG. 4 illustrates heat production by Sauromatum guttatum appendix treated with aspirin (ASA) and various opioid peptides and the neurotoxic peptide β-amyloid peptide (Aβ 1-42).

FIG. 5 illustrates heat production by Sauromatum guttatum appendix treated with salicylic acid (SA) in the presence of human opioid peptides (β-Endorphin and Neuropeptide AF) and, β-amyloid peptide, (Aβ 1-42); and a plant virulent bacterial pathogen (Pst DC3000).

FIG. 6 illustrates heat production by Sauromatum guttatum appendix treated with 2,6-dihydroxybenzoic acid (2,6-DHBA) in the presence of β-amyloid peptide (Aβ 1-42), SEQ ID NO: 2, and SEQ ID NO: 1.

FIG. 7 illustrates effects of SEQ ID NO: 1 on 3-hour post-surgical pain in rats.

FIG. 8 illustrates effects of SEQ ID NO: 1 on 3-day post-surgical pain in rats.

### DETAILED DESCRIPTION OF INVENTION

The present invention relates to compositions and methods for modulating conditions in plants and/or animals. Such conditions include mitochondrial related or metabolic related conditions, pain, plant pathogens, infections, fever, Alzheimer's disease, etc.

In one aspect, a composition herein includes a polypeptide comprising, consisting essentially of, or consisting of any one or more of: SEQ ID NOs: 1-349, or any one or more of: SEQ ID NOs: 1-24, 50-244, 248-249, or 257-349, or any one or more of: SEQ ID NOs: 1-2, 153, or 304-349 or any one or more of SEQ ID NO: 1 or 153 or 304 or 305.

In one aspect, a composition comprises a nucleic acid sequence encoding one or more of the above.

In one aspect, a composition comprises an antibody that specifically binds an epitope comprising one or more of the above polypeptides.

In one aspect, the present invention relates to a method for identifying novel compositions (e.g., polypeptides, peptide nucleic acids, nucleic acids, and small molecules) that modulate conditions in plants or animals as described herein (e.g., pain, fever, neurodegenerative conditions, metabolic conditions, etc.) Such methods include administering a test agent to a thermogenic plant; measuring temperature of said thermogenic plant; and determining if said test agent modulates temperature in said plant.

In one aspect, the compositions herein are used to treat a mitochondrial or metabolic condition selected from the group consisting of: innate immune response activation and ability to fight parasites and pathogens, pain, inflammation, temperature regulation, neoplastic growth (e.g., cancer), skin and dermatological conditions, and neurological and neurodegenerative conditions.

### DEFINITIONS

The term “agonist” as used herein refers to any compound, small molecule, or agent, or a peptide that stimulates a biological activity. Examples of agonists include, but are not limited to, antibodies, antisense nucleic acids, siRNA nucleic acids, and other binding agents. Such agents can stimulate receptors, e.g., morphine antagonist of the opiate μ receptors.

The term “amino acid” or “amino acid residue” refers to an amino acid, which is preferably in the L-isomeric form. When an amino acid residue is part of a polypeptide chain, the D-isomeric form of the amino acid can be substituted for the L-amino acid residue, as long as the desired functional property is retained. NH₂ refers to the free amino group present at the amino terminus of a polypeptide. COOH refers to the free carboxyl group present at the carboxyl terminus of a polypeptide. The amino acids herein can be represented by their standard 1-letter code or 3-letter code. An amino acid residue represented by “X” or “Xxx” refers to any one of the naturally occurring or non-naturally occurring amino acid residues known in the art or to a modification of a nearby residue. In keeping with standard protein nomenclature described in J. Biol. Chem., 1969, 247:3552-59, and adopted at 37 C.F.R. §§1.821-2461.822, all amino acid residue sequences represented herein by formulae have a left to right orientation in the conventional direction of amino-terminus to carboxyl-terminus. In addition, the phrase “amino acid residue” is broadly defined to include modified and unusual amino acids, such as those referred to in 37 C.F.R. §§1.821-1.822, and incorporated herein by reference. In a peptide or polypeptide, suitable conservative substitutions of amino acids are known to those of skill in this art and can be made generally without altering the biological activity of the resulting molecule. Watson et al., book (1987, Molecular Biology of the Gene, 4th Edition, The Benjamin Cummings Pub. Co., p. 224), is incorporated herein by references. Amino acid substitutions are typically of single residues, such substitutions are preferably made with those set forth in Table I., but may be of multiple residues, either clustered or dispersed. An amino acid can be replaced with a different naturally occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size. Additions encompass the addition of one or more naturally occurring or non-conventional amino acid residues. Deletion encompasses the deletion of one or more amino acid residues.

TABLE I
Conservative amino acid substitution

| Original residue | Conservative substitution(s) |
|---|---|
| Ala | Gly; Ser |
| Arg | Lys |
| Asn | Gln; His |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr, Ile |
| Phe | Met; Leu; Tyr |
| Ser | Thr |
| Thr | Ser |
| Trp | Tyr |
| Tyr | Trp; Phe |
| Val | Ile; Leu |

Substitutions encompassed by the present invention may also be “non-conservative”, in which an amino acid residue which is present in a peptide is substituted with an amino acid having different properties, such as naturally-occurring amino acid from a different group (e.g., substituting a charged or hydrophobic amino acid with alanine), or alternatively, in which a naturally-occurring amino acid is substituted with a non-conventional amino acid.

The term “analog(s)” as used herein refers to a composition that retains the same structure or function (e.g., binding to a receptor) as a polypeptide or nucleic acid herein, such as the same gene from a different organism. Examples of analogs include mimetics or peptidomimetics, peptide, nucleic acids, small and large organic or inorganic compounds, as well as derivatives and variants of a polypeptide or nucleic acid herein. Such derivatives and variants refer to peptides and nucleic acids that differ from the naturally occurring polypeptides and nucleic acids by one or more amino acid or nucleic acid deletions, additions, substitutions or side-chain modifications. In some embodiments, a peptide analog is a peptide in which one or more of the amino acids has undergone side-chain modifications. Examples of side-chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄. In some embodiments, a peptide analog is one in which the guanidine group of arginine residue(s) is modified by the formation of heterocyclic condensation products with reagents such as 2,3-butanedione, phenylglyoxal and glyoxal; carboxyl group(s) is modified by carbodiimide activation via O-acylisourea formation followed by subsequent derivitisation, for example, to a corresponding amide; sulphydryl group(s) may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH. In any of the analogs herein, any modification of cysteine residues preferably do not affect the ability of the peptide to form the necessary disulphide bonds. In some embodiments, a peptide analog comprises tryptophan residue(s) that are modified by, for example, by oxidation with N-bromosuccinimide or alkylation of the indole ring with 2-hydroxy-5-nitrobenzyl bromide or sulphenyl halides; tyrosine residues altered by nitration with tetranitromethane to form a 3-nitrotyrosine derivative; imidazole ring(s) of a histidine residue modification accomplished by alkylation with iodoacetic acid derivatives or N-carbethoxylation with diethylpyrocarbonate; proline residue(s) modified by, for example, hydroxylation in the 4-position; glycosylation variants from a completely unglycosylated molecule to a modified glycosylated molecule; and altered glycosylation patterns as a result from expression of recombinant molecules in different host cells.

The term “antagonist” as used herein refers to any compound, small molecule, or agent, a peptide that inhibits or reduces a biological activity. Examples of antagonist molecules include, but are not limited to, peptides, small molecules, antibodies, antisense nucleic acids, siRNA nucleic acids, and other binding agents.

The term “antibody” is used in the broadest sense and specifically covers, for example, polyclonal antibodies, monoclonal antibodies (mAbs) (including agonist, antagonist, and neutralizing antibodies), chimeric antibodies, antibody compositions with mono and polyepitopic specificity, single chain antibodies, anti-idiotypic (anti-Id) antibodies to antibodies that can be labeled in soluble or bound form, polymers and conjugates of immunoglobulins, as well as fragments, regions or derivatives thereof (e.g., separate heavy chains, light chains, Fab, F(ab′)2, Fabc, and Fv). Antibody fragments can be prepared for example by enzymatic cleavage of antibodies with enzymes such as pepsin or papain. Antibody aggregates, polymers and conjugates can be generated by diverse methods, e.g. by thermal treatment, reaction with substances such as glutaraldehyde, reaction with immunoglobulin-binding molecules, biotinylation of antibodies and subsequent reaction with streptavidin or avidin. The term “monoclonal antibody” as used herein refers to an antibody obtained from a population of substantially homogeneous antibodies, i.e., the individual antibodies comprising the population are identical except for possible naturally-occurring mutations that may be present in minor amounts.

The term “antigens” includes monovalent and polyvalent antigens. A polyvalent antigen is a molecule or a molecule complex to which simultaneous binding of more than one immunoglobulin is possible, whereas a monovalent antigen can bind only a single antibody at each particular time. Hapten is normally the designation given to a molecule which is not immunogenic per se but which is normally bound to a carrier for immunization purposes.

The term “effective amount” as used herein when referring to a composition means the amount or dosage of that composition that is required to induce a desired effect. In some embodiments, an effective dose refers to an amount that is required to induce a local analgesic, anti-pyrogenic, flowering, pesticide, anti-dementia, and/or anti-inflammatory effect.

The term “fragment” as used herein refers to a portion of a composition. For example, when referring to a polypeptide, a fragment of a polypeptide is some but not the entire amino acid polymer that comprises the polypeptide. A polypeptide fragment can have up to 99, 95, 90, 85, 80, 75, 70, 65, or 60% of the sequence of the parent polypeptide. In some embodiments, a fragment has between 3-40, 3-30, 4-20, or 4-10 amino acids of the parent sequence.

The terms “gene therapy” and “genetic therapy” refer to the transfer of heterologous nucleic acids to the certain cells, target cells, of a mammal, particularly a human, with a disorder or conditions for which such therapy is sought. The nucleic acid is introduced into the selected target cells in a manner such that the heterologous DNA is expressed and a therapeutic product encoded thereby is produced. Alternatively, the heterologous nucleic acids can in some manner mediate expression of a nucleic acid that encodes the therapeutic product; it can encode a product such as a peptide or RNA that in some manner mediates, directly or indirectly, expression of a therapeutic product. Genetic therapy can also be used to nucleic acid encoding a gene product replace a defective gene or supplement a gene product produced by the mammal or the cell in which it is introduced.

The term “homolog” when referring to a polymer (e.g., a peptide or a nucleic acid) refers to a second polymer that has at least about 50 sequence identity, at least 55% sequence identity, at least 60% sequence identity, at least 65% sequence identity, at least 70% sequence identity, at least 55% sequence identity, at least 80% sequence identity; or at least about 81% sequence identity, at least about 82% sequence identity, or at least about 83% sequence identity, or at least about 84% sequence identity, or at least about 85% sequence identity, or at least about 86% sequence identity, or at least about 87% sequence identity, or at least about 88% sequence identity, or at least about 89% sequence identity, or at least about 90% sequence identity, or at least about 91% sequence identity, or at least about 92% sequence identity, or at least about 93% sequence identity, or at least about 94% sequence identity, or at least about 95% sequence identity or at least about 96% sequence identity, or at least about 97% sequence identity, or at least about 98% sequence identity or at least about 99% sequence identity and preferably the same function. For example, a polypeptide homologous to any of the polypeptides herein (e.g., SEQ ID NOs: 1-305) is one that can have at least 80% sequence identity and similar function of modulating pain or fever, or more preferably acting as an agonist or antagonist for pain receptors.

The term “isolated” means altered from its natural state; i.e., if it occurs in nature, it has been changed or removed from its original environment, or both. For example, a naturally occurring polynucleotide or a polypeptide naturally present in a living animal in its natural state is not “isolated”, but the same polynucleotide or polypeptide separated from the coexisting materials of its natural state is “isolated”, as the term is employed herein. For example, with respect to polynucleotides, the term isolated means that it is separated from the nucleic acid and cell in which it naturally occurs.

The term “protein”, “peptide”, “oligopeptides” or “polypeptide” as used herein refers to any composition that includes two or more amino acids joined together by a peptide bond. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (GPI) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide (e.g., any one or more of SEQ ID NO: 1-349 or any one or more of SEQ ID NO: 1, 153, or 304-349), and a spacer. A DNA-construct encoding the peptide and a spacer alternate repeats can be synthesized using methods known in the art. (Rötzschke et al., 1997, Proc. Natl. Acad. Sci. USA 94:14642-14647). The above methods allow for the amplification of the antigenicity of the peptide and for insertion into an expression vector at high levels.

The term “opioid” as used herein means all agonists and antagonists of opioid receptors, such as mu (μ), kappa (κ), and delta (δ) opioid receptors and subtypes thereof. For a discussion of opioid receptors and subtypes see Goodman and Gilman's The Pharmacological Basis of Therapeutics 9th ed. J. G. Harman and L. E. Limird Eds., McGraw-Hill New York, 1996, pp. 521-555, which is incorporated herein by reference for all purposes. The opioid can be any opioid receptor agonist or antagonist known or to be developed. Preferred opioids interact with the μ-opioid receptor or the κ- and δ-opioid receptors. Preferably, the opioid is an opioid-receptor agonist or antagonist.

The term “organism” as used herein can be, for example, a microorganism (e.g., virus or bacteria), plant (e.g., crop plants such as soy, wheat, barley, rice, corn, sugar, etc.), or animal. Animals include both mammals (e.g., farm animals, donkeys, goats, chicken, rabbits, dogs, cats, swine, cattle, sheep, horses, or primates) and non-mammals, e.g., (e.g., insects and birds). In some cases the animal is a mammal, or a human.

The term “purified” as used herein to describe a polypeptide, polynucleotide, or other compositions, refers to such polypeptide, polynucleotide, or other composition separated from one or more compounds, which are usually associated with it in nature. Such other compositions can be, for example, other polypeptides or polynucleotides, carbohydrates, lipids, etc. The term “purified” can also be used to specify the separation of monomeric polypeptides of the invention from oligomeric forms such as homo- or hetero-dimers, trimers, etc. The term “purified” may also be used to specify the separation of covalently closed (i.e. circular) polynucleotides from linear polynucleotides. A substantially pure polypeptide or polynucleotide typically comprises at least about 50%, 60%, 70%, 80%, or 90% weight/weight of a polypeptide or polynucleotide sample, or at least about 95%, 96%, 97%, 98%, 99%, or 99.5% weight/weight of a polypeptide or polynucleotide sample. As a preferred embodiment, a polypeptide or polynucleotide of the present invention is at least 10%, 20%, 30%, 40%, 50%, 60%, 70%, 80%, 90%, 95%, 96%, 96%, 98%, 99%, or 100% pure relative to heterologous polypeptides and polynucleotides, respectively.

### Compositions

The compositions herein can include any one or more of the polypeptides, nucleic acids, antibodies, or small molecules described herein or any homolog, analog, prodrug, metabolite, salt, or polymorph thereof. The polymorphs contemplated herein are preferably the most stable crystalline form of the composition. For example, the polymorph may be water-soluble. Polymorphs of any of the compositions herein can be detected using any method know in the art such as in vitro testing, where the material is crystallized by different methods. Once a crystal is created, its water solubility is determined and the most thermodynamically stable polymorph is selected.

A composition herein can include an isolated polypeptide comprising, consisting essentially of, or consisting of SEQ ID NOS: 1-349, or any fragment thereof, provided that such polypeptide does not include any or all of SEQ ID NOS: 350-371. Thus, a composition herein could include an isolated polypeptide consisting essentially of FLPS (SEQ ID NO: 1) but specifically excluding any or all of SEQ ID NOS: 350-371 or comprising of FLPS (SEQ ID NO: 1) but being not longer than 100, 50, 30 or 20 amino acids and specifically excluding any or all of SEQ ID NOS: 350-371. In some instances a composition herein comprises an isolated peptide having no more than 100, 50, 30, 20, or 10 amino acids wherein the peptide includes FLPS in its sequence and the peptide specifically excludes any or all of SEQ ID NOS: 350-371. A polypeptide herein can further comprise or consist essentially of FLPS based on any of the other characteristics described herein, provided that the complete polypeptide is not any or all of SEQ ID NOS: 350-371. A polypeptide herein can further consist of FLPS and 0, 1, 2, 3, 4, 5, 6, 7, 8, 9, or 10 additional amino acids, but specifically exclude any or all of SEQ ID NOS: 350-371.

In some embodiments, the invention herein contemplates improving, alleviating, preventing or treating a condition in a plant, wherein the condition is associated with metabolism, heat production, or pathogen resistance by modulating (e.g., by expressing recombinantly or otherwise, or by inhibiting or antagonizing) any one or more of SEQ ID Nos: 1-349, or any one or more of SEQ ID NO: 1, 153, 304-349; or by modulating any one or more of SEQ ID NOS: 245, 246, 247, 253 or any fragment thereof or complex thereof or a protein or complex comprising SEQ ID NOS: 245, 246, 247, 253. The present invention also contemplates alleviating, preventing or treating a condition in a plant by administering to the plant a composition comprising any one or more of SEQ ID Nos: 1-349, or any one or more of SEQ ID NO: 1, 153, or 304-349 or a nucleic acid encoding the same, or an antibody that selectively bind the above, or a small molecule that mimics any of the above or a polymorph of any of the above. The condition of the plant can be, for example, one that is associated with metabolism, heat production, or pathogen resistance.

In some embodiments, the invention herein contemplates improving, alleviating, preventing or treating a condition in an animal, such as a human, wherein the condition can be, e.g., pain, fever, Alzheimer's disease, or a metabolic condition, or in plants, wherein the condition can be, e.g., temperature, resistance to pathogen, and a metabolic condition, by modulating (e.g., using gene therapy, selectively binding using antibodies) antagonizing, or administering as a therapeutic a composition comprising any one or more of SEQ ID NOS: 1-349, or any one or more of SEQ ID NOS: 1-24, 50-152, 248-250, 254-349, or any one or more of SEQ ID NO: 1, 153, 304-349 or any fragment thereof, or complex thereof, or a polymorph of any of the above or a small molecule equivalent to any of the above. (For example, polypeptides such as SEQ ID NO: 250 and all other peptides described herein from human can be genetically engineered into a vector and transplanted into plants.)

A polypeptide herein can further comprise additional amino acid residues in its C-terminus and/or N-terminus For example, the present invention contemplates any one or more of SEQ ID NOS 1-349 or any one or more of SEQ ID NOS: 1, 153, 304-349 further comprising one or more amino acids such as, e.g., KFLPS (SEQ ID NO: 257), FLPSI (SEQ ID NO: 258), RFLPS (SEQ ID NO: 259) or FLPSE (SEQ ID NO: 260). Thus, a polypeptide herein can be a 2-mer (e.g., Lys-Pro, Pro-Lys, Phe-Lys, Lys-Phe, Ser-Ser, Pro-Ser, Ser-Pro, Ser-Leu), a 3-mer (e.g., part of any of the polypeptides herein, such as the middle portion thereof), 4-mer, 5-mer, 6-mer, 7-mer, 8-mer, 9-mer, 10-mer, 11-mer, 12-mer, 13-mer, 14-mer, 15-mer, or larger, and may be up to 40, 30, 20, or 10 amino acids long.

In some cases, the reverse sequences of any of the polypeptides herein can be used. In alternative or in addition to the above, any of the polypeptides herein can comprise one or more D-amino acids. For example, polypeptides of the invention include those comprising, consisting essentially of, or consisting of: any one or more of SEQ ID NOs: 1-349, or any one or more of SEQ ID NO: 1, 153, 304-349; or any one or more of SEQ ID NO: 1-152, 245-256, or 257-349, wherein at least one of the amino acids is a D-amino acid. In some cases the D-amino acids are the first 2 amino acids from the N-terminus. In some cases, the D-amino acids are the first three amino acids from the N-terminus. In some cases all of the amino acids are D-amino acids.

In some embodiments, a composition comprises a polypeptide comprising, consisting essentially of, or consisting of any one or more of SEQ ID NOs: 1-349, or any one or more of SEQ ID NOS: 1-14, 50-152, 248-249, 257-349, or any one or more of SEQ ID NOs: 1, 2, 153, 304-349, or any homolog, analog, prodrug, metabolite, or fragment thereof, or any salt thereof, small molecule thereof, nucleic acid encoding thereof, antibody thereof, or polymorph thereof.

In some embodiments, the compositions herein include a polypeptide having the reverse amino acid sequence of any of the above amino acid sequences, such as e.g., SEQ ID NOs: 1-349 or any one or more of SEQ ID NOS: 153-244, or 280-349 or a homolog, analog, prodrug, metabolite, or fragment thereof, or salt thereof, or small molecule that mimics any of the above, or a nucleic acid encoding any of the above, or antibody that selectively binds any of the above, or a polymorph of any of the above.

In some embodiments, the compositions herein include a peptide having the reverse amino acid sequence of any of the above amino acid sequences, such as SEQ ID NOs: 245-247 or any homolog, analog, prodrug, metabolite, or fragment thereof, or any salt thereof, small molecule thereof, nucleic acid encoding thereof, antibody thereof, or polymorph thereof. For example, a composition herein can comprise a peptide fragment of SEQ ID NO: 245, 246, and/or 247 which is between 3-50, 3-40, 3-30, 3-20, or 3-10 amino acids in length. In some cases, such fragments can have at least one or at least two phenylalanines. In some cases, such peptide fragments can comprise the sequence Phe-Leu-Pro-Ser (SEQ ID NO: 1). Any of the peptides herein are contemplated in both their forward and reverse sequences. SEQ ID NOs: 152-244, 280-298-, are the reverse sequences of SEQ ID NOs: 1-14 and 50-151, 257-279.

In another aspect, the present invention relates to nucleic acids that encode any of the above peptides and antibody that specifically bind any of the above polypeptides.

In some embodiments, the compositions (e.g., polypeptides) herein are used to modulate the effects of SEQ ID NO: 49 or to reduce the effects or treat or prevent Alzheimer's disease.

In some embodiments, the above compositions (e.g., polypeptides) can be used modulate or enhance the effects of SEQ ID NOs: 15-48, 248-249.

In some embodiments, the above compositions (e.g., polypeptides) can be used to create a library to analyze functionality of compounds and compositions (e.g., small molecules) that regulate mitochondria activities.

In some embodiments, a composition herein comprises a small molecule that mimics (e.g., has similar 3 D structure or has similar biological activity) as any of the polypeptides herein.

In some embodiments, a composition herein comprises a polymorph of any of the polypeptides or small molecules herein.

In some embodiments, the compositions herein comprise a salt of any of the above.

In some embodiments, the above compositions (e.g., peptides) can be used for screening antagonists, agonists, and modulators of different mitochondrial activities.

1. Polypeptides

In some aspects a composition herein comprises, consists essentially of, or consists of one or more of the polypeptides described herein such as those that comprise, consist essentially of, or consist of any one of amino acid sequences of any one or more of SEQ ID NO: 1-349 or an analog, salt, polymorph, metabolite, or prodrug thereof or any one or more of SEQ ID NO: 1-244, 248-249, or 257-349 or an analog, salt, polymorph, metabolite, or prodrug thereof; or sequences which are the reverse of the above or a homolog, analog, salt, prodrug, fragment, or metabolite of the above, or a polymorph of any of the above, or a combination thereof.

In some cases, a composition herein comprises a polypeptide comprising any one or more of SEQ ID NO: 1-349. In some cases such polypeptide has up to 10,000, 1,000, 100, 90, 80, 70, 60, 50, 40, 30, 20, 10, 9, 8, 7, 6, 5, 4, or 3 amino acids. The invention herein also contemplates analogs, salts, polymorphs, metabolites, or prodrugs thereof.

In some cases, a composition comprises of a polypeptide that consists essentially of any one or more of SEQ ID NO: 1-349. In some cases such polypeptide has up to 10,000, 1,000, 100, 90, 80, 70, 60, 50, 40, 30, 20, 10, 9, 8, 7, 6, 5, 4, or 3 amino acids. The invention herein also contemplates analogs, salts, polymorphs, metabolites, or prodrugs thereof.

In some cases, a composition comprises of a polypeptide that consists of any one or more of SEQ ID NO: 1-349. In some cases such polypeptide has up to 10,000, 1,000, 100, 90, 80, 70, 60, 50, 40, 30, 20, 10, 9, 8, 7, 6, 5, 4, or 3 amino acids. The invention herein also contemplates analogs, salts, polymorphs, metabolites, or prodrugs thereof.

In some cases, a composition includes more than 1, 2, 3, 4, 5, or 6 of the polypeptides described herein or an analog, salt, polymorph, metabolite, or prodrug thereof.

The invention herein also contemplates compositions comprising peptides such as Leu-Pro and Pro-Leu (e.g., Xxx-Leu-Pro, Xxx-Pro-Leu, Leu-Pro-Xxx, Pro-Leu-Xxx, Xxx-Leu-Pro-Xxx, and Xxx-Pro-Leu-Xxx, as well as longer peptides comprising any of the above sequences). In such embodiments, Xxx can be Phe, Ser, Trp, Tyr, or Pro, for example. Other peptides contemplated herein include: Phe-Leu, Leu-Phe, Pro-Ser, or Ser-Pro and peptides that comprise or consist essentially any of the above.

For example, a herein can comprise, consist essentially of, or consist of a peptide comprising dipeptide Pro-Leu conservative substitution dipeptide thereof, or or a homolog, derivative, or analog thereof, or a small molecule thereof, or a salt, metabolite, or prodrug thereof, or a nucleic acid encoding such peptide; and a pharmaceutical excipient.

The present invention also contemplates a composition comprising, consisting, or consisting essentially of: a peptide comprising Xxx-Leu-Pro or a homolog or analog thereof, or a small molecule thereof, or a salt, metabolite, or prodrug thereof, or a nucleic acid encoding such peptide; and a pharmaceutical excipient. In some cases, Xxx is an aromatic amino acid or a derivative or analog thereof. For example, in some cases Xxx is Tyr or Phe or Trp or His or Pro or a conservative substitution of any of the above, or an analog or a derivative of any of the above.

The present invention also contemplates a composition comprising, consisting, or consisting essentially of: a peptide comprising Xxx-Leu-Phe or a homolog or analog thereof, or a small molecule thereof, or a salt, metabolite, or prodrug thereof, or a nucleic acid encoding such peptide; and a pharmaceutical excipient. In some cases, Xxx is an aromatic amino acid or a derivative or analog thereof. For example, in some cases Xxx is Tyr or Phe or Trp or His or Pro or a conservative substitution of any of the above, or an analog or a derivative of any of the above.

The present invention also contemplates a composition comprising, consisting, or consisting essentially of: a peptide comprising Leu-Pro-Xxx or a homolog or analog thereof, or a small molecule thereof, or a salt, metabolite, or prodrug thereof or a nucleic acid encoding such peptide; and a pharmaceutical excipient. In some cases Xxx is an amino acid such as Tyr, Thr, Glu, Asp or Ser or a conservative substitution of any of the above, or an analog or a derivative of any of the above.

The present invention also contemplates a composition comprising, consisting, or consisting essentially of: a peptide comprising Ser-Leu-Xxx or a homolog or analog thereof, or a small molecule thereof, or a salt, metabolite, or prodrug thereof, or a nucleic acid encoding such peptide; and a pharmaceutical excipient. In some cases, Xxx is an aromatic amino acid or a derivative or analog thereof. For example, in some cases Xxx is Tyr or Phe or Trp or His or Pro or a conservative substitution of any of the above, or an analog or a derivative of any of the above.

The present invention also contemplates a composition comprising, consisting, or consisting essentially of: a peptide comprising Xxx-Leu-Pro-Xxx or a homolog or analog thereof, or a small molecule thereof, or a salt, metabolite, or prodrug thereof, or a nucleic acid encoding such peptide; and a pharmaceutical excipient. In some cases, each of Xxx can be Tyr or Phe or Trp or His or Pro or an analog or a derivative thereof. In some cases, each of Xxx can be Phe or Ser or a conservative substitution of any of the above, or an analog or a derivative of any of the above.

The present invention also contemplates a composition comprising, consisting, or consisting essentially of: a peptide comprising Xxx-Pro-Leu-Xxx or a homolog or analog thereof, or a small molecule thereof, or a salt, metabolite, or prodrug thereof, or a nucleic acid encoding such peptide; and a pharmaceutical excipient. In some cases, each of Xxx can be Tyr or Phe or Trp or His or Pro or an analog or a derivative thereof. In some cases, each of Xxx can be an aromatic amino acid or a derivative or analog thereof. For example, in some cases Xxx is Tyr or Phe or Trp or His or Pro or a conservative substitution of any of the above, or an analog or a derivative of any of the above. In some cases, Xxx is Ser or a conservative substitution of Ser, or an analog or a derivative of Ser.

The present invention also specifically contemplates pharmaceutical formulation comprising a composition comprising, consisting, or consisting essentially of tripeptides such as: Phe-Leu-Pro, Trp-Leu-Prp, Tyr-Leu-Pro, Pro-leu-Phe, Pro-Leu-Trp, and Pro-Leu-Tyr. Any of the above amino acids can be substituted by a conservative substitution, or an analog or derivative of any of the above. The invention also contemplates non-linear (e.g., cyclic) forms of the above and small molecules equivalent to any of the above.

In any of the embodiments described herein a composition (e.g., peptide) can be linear or non-linear (e.g., cyclic). Thus, any one of SEQ ID NOS: 1-349 can be made cyclical. Examples of cyclic peptides contemplated include: cyclo(Xxx-Leu-Pro-Ser), wherein Xxx can be any amino acid or wherein Xxx is Phe, Pro, Ser, Tyr, Trp or His, cyclo(Phe-Leu-Pro-Ser), cyclo(Tyr-Leu-Pro-Ser), cyclo(Trp-Leu-pro-Ser), cyclo(Ser-Pro-Leu-Phe), cyclo(Ser-Pro-Leu-Tyr), and cyclo(Ser-Pro-leu-Trp). Thus, a composition herein can comprise, consist, or consist essentially of a cyclic peptide (e.g., a cyclic peptide of any of the peptides herein such as SEQ ID NO: 1-349) or an analog (e.g., small molecule) thereof or salt, prodrug, or metabolite thereof. In some cases, a composition comprises a non-linear (e.g., cyclic) peptide of SEQ ID NO: 1, 153, 304-349 or an analog thereof.

Any of the peptides described herein can have one or more amino acids replaced by another naturally occurring or non-naturally occurring amino acid, preferably having similar charge, 3 D structure, or by a conservative substitutions.

Any of the peptides herein can further be modified by one or more of the following modifications: acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (GPI) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination

Furthermore, any of the peptides herein can have one or more D-amino acids or one or more L-amino acids. In some cases, a peptide has at least 1, 2, 3, 4, or 5 D amino acids or at least 1, 2, 3, 4, or 5 L-amino acids.

As further described herein, compositions herein include those comprising any of the above peptides and small molecules equivalent thereof and homologs and analogs thereof, and polymorphs thereof, and fragments thereof, as well as prodrugs, metabolites and salts thereof. Such compositions can be used to treat or prevent a condition in a plant or an animal. In some cases, such compositions are used to treat a condition in a human. Such a condition can be e.g., pain, a neurological or neurodegenerative condition (e.g., Alzheimer's), inflammation, addiction, a metabolic disorder, neoplastic growth, or fever. In some cases, such compositions are used to treat a plant infected by a pathogen or to prevent infection of a plant by a pathogen.

In some embodiments, a composition comprises more than 1, 2, 3, 4, and 5 of the polypeptides above.

The polypeptides herein may be created synthetically by any means known in the art (synthetically synthesized or using recombinant DNA technology). In some embodiments, they may include an additional methionine at the N-terminus (e.g., SEQ ID NO: 105, MFAGYFAG) or an N-terminus methionine may be included on to them.

In some embodiments, a polypeptide herein has up to about 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, 5, or 4 amino acids residue; or at least about 3, 4, 5, 6, 7, 8, 9, 10, 11, 12, 12, 13, 14, 15, 16, 17, 18, 19, 20, 25, 30, 35, 40, 45, or 50, amino acid residues; or between about 2-50, 2-40, 2-30 or 2-10 amino acid residues. In some embodiments, a polypeptide herein has up to 500,000, 100,000, 75,000, 50,000, 15,000, 14,000, 13,000, 12,000, 11,000, 10,000, 9,000, 8,000, 7,000, 6,000, 5,000, 4,000, 3,000, 2,000, 1,000, 900, 800, 700, 600, 500, 400, 300, or 200 Daltons. In some embodiments, a polypeptide herein has between 200-200,000, 300-100,000, 400-50,000, or 500-1000 Daltons.

The polypeptides herein are preferably isolated such that it is free of other compounds or molecules that it normally is associated with in vivo. For example, an isolated peptide of the invention can constitute at least about 50%, about 55%, about 60%, about 65%, about 70%, about 75%, about 80%, about 85%, about 90%, about 95%, or about 99% w/w of a sample containing it. In some embodiments, a polypeptide of the invention is purified.

In some embodiments, a polypeptide of the present invention can be present in different conformational states (e.g., extended, single β-bend, and double β-bend (Deschamps, 2005)). The design of potent and selective opioid peptides and small analgesic drugs have involved the application of both conformational and topographical constraints (e.g., unsubstituted Gly 3 residue in of enkephalin can be replaced with d-Ala, or mercaptoproline, 21 or removing the Gly 3 residue to form a more rigid cyclic tetrapeptide (Deschamps, 2005, AAPS J. 7:E813; Hashimoto et al., 2002, Bioorg. Med. Chem. 10:3319; Salvadorii et al., 2002, BBRC, 223:640).

In some embodiments, a polypeptide of the present invention present in different oligomerization states as a result of formulation in saline and other formulations required for bioactivity e.g., aggregations of beta-amyloid peptide (Murphy 2002, or self assembly of peptides (Ulrich et al., 1999).

Polypeptides in different oligomerization states (or not oligomerized) can be used as backbone for cyclization and modification to constrain the conformation state of the bioactive peptide. In some embodiments, a polypeptide herein is modified or adapted for slow-release. Such modification can include substitution of one or more, 2 or more, 3 or more, or 4 or more amino acids residues from an L-amino acid residue to a D-amino acid residue. In some embodiments at least about 10%, 20%, 30%, 40%, 50%, 60%, 70%, 80%, 90% or 100% of the residue in a polypeptide are D-amino acids.

In some embodiments, a polypeptide of the present invention includes one or more post-translational modifications, e.g., N-linked or O-linked carbohydrate chains, processing of N-terminal or C-terminal ends, attachments of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the polypeptide.

Also provided by the invention are chemically modified derivatives of the polypeptides of the invention, which may provide additional advantages such as increased solubility, stability and circulating time of the polypeptide, or decreased immunogenicity. See U.S. Pat. No. 4,179,337. The chemical moieties for derivitization may be selected. See, U.S. Pat. No. 4,179,337, which is hereby incorporated by reference in its entirety. The chemical moieties for derivitization may be selected from water-soluble polymers such as polyethylene glycol (PEG), copolymers, carboxymethylcellulose, dextran, polyvinyl alcohol and the like. Such derivitization may occur at random positions within the molecule, or at predetermined positions within the molecule and may include one, two, three or more attached chemical moieties.

For example, in some embodiments the chemical moiety used for derivitization may be a polymer of any molecular weight, and may be branched or unbranched. If PEG is used for derivitization, the preferred molecular weight is between about 1 kDa and about 100 kDa (the term “about” indicating that in preparations of PEG, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the PEG to a therapeutic polypeptide or analog).

The PEG molecules (or other chemical moieties) should be attached to the polypeptide with consideration of effects on functional or antigenic domains of the polypeptide. There are a number of attachment methods available to those skilled in the art, e.g., EP 0 401 384, (coupling PEG to Granulocyte Colony Stimulating Factor (G-CSF)), Malik et al., (1992), Exp. Hematol. 20:1028-1035. This article reports on pegylation of Granulocyte/Macrophage Colony Stimulating Factor (GM-CSF) using tresyl chloride, which its disclosures is hereby incorporated by reference in its entireties. For example, PEG may be covalently bound through amino acid residues via a reactive group, such as, a free amino or carboxyl group. Reactive groups are those to which an activated PEG molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the PEG molecules. Preferred for therapeutic purposes is attachment at an amino group, such as attachment at the N-terminus or lysine group.

The polypeptides herein can be chemically modified at the N-terminus. Using PEG as an illustration of the present composition, one may select from a variety of PEG molecules (by molecular weight, branching, etc.), the proportion of PEG molecules to polypeptide (polypeptide) molecules in the reaction mix, the type of pegylation reaction to be performed, and the method of obtaining the selected N-terminally pegylated polypeptide. The method of obtaining the N-terminally pegylated preparation (i.e., separating this moiety from other monopegylated moieties if necessary) may be by purification of the N-terminally pegylated material from a population of pegylated polypeptide molecules. Selective polypeptides chemically modified at the N-terminus modification may be accomplished by reductive alkylation, which exploits differential reactivity of different types of primary amino groups (lysine versus the N-terminal) available for derivitization in a particular polypeptide. Under the appropriate reaction conditions, substantially selective derivitization of the polypeptide at the N-terminus with a carbonyl group containing polymer is achieved.

In any of the embodiments herein, a polypeptide of the present invention may be modified in its N-terminus. Examples of N-terminus modifications include an N-terminus methionine, N-terminus signal peptide, or a prosequence. An N-terminus methionine may be used for expression of a polypeptide recombinantly. A “signal sequence” or “presequence” refers to any sequence of amino acids bound to the N-terminal portion of a polypeptide herein (e.g., an amino acid sequence selected from SEQ ID NOs: 1-349, or any homolog or analog thereof), which may participate in the secretion of the polypeptide. The term “prosequence” as used herein refers to a sequence of amino acids bound to the mature form of a polypeptide herein (e.g., an amino acid sequence selected from the group consisting of SEQ ID NOs: 1-349, or any homolog or analog thereof), which when removed results in the appearance of the “mature” form of the polypeptide (e.g., an amino acid sequence selected from the group consisting of SEQ ID NOs: 1-349, or any homolog or analog thereof). Preferably, a prosequence is autocleaved/cleaved by naturally occurring enzymes, which are found at an area in which the mature polypeptide needs to be active.

In some embodiments a polypeptide herein includes one or more conservative substitutions. Such substitutions are selected from the Table I. Other known conserved substitutions may also be known to a person of ordinary skill in the art.

In some embodiments, a polypeptide of the present invention is modified to be more resistant to proteolysis. For example, a polypeptide comprising, consisting essentially of, or consisting of an amino acid sequence of (or encoded by) any one or more of SEQ ID NOs: 1-349, (any of the above with a methionine at the N-terminus) or any homolog, analog or fragment thereof may include one or more peptide bonds in which the —CONH— peptide bond is modified and replaced by a non-cleavable bond, e.g., a (CH₂NH) reduced bond, a (NHCO) retro inverso bond, a (CH₂—O) methylene-oxy bond, a (CH₂—S) thiomethylene bond, a (CH₂CH₂) carba bond, a (CO—CH₂) cetomethylene bond, a (CHOH—CH₂) hydroxyethylene bond), a (N—N) bound, a E-alkene bond or also a —CH—CH-double bond.

In some embodiments, a polypeptide sequence herein is constructed in its reverse sequence to prevent degradation. Examples of reverse sequence include those of SEQ ID NO: SEQ ID NOs: 153-244, 280-298.

2. Nucleic Acids

The present invention also provides for a nucleic acid that encodes any of the polypeptides described herein. For example, in some embodiments, the present invention relates to a nucleic acid sequence that encodes a polypeptide comprising, consisting essentially of, or consisting of an amino acid sequence such as any one or more of SEQ ID NOs: 1-349, or any one or more of SEQ ID NOs: 1-244, 248-249, 257-349, or any one or more of SEQ ID NOs SEQ ID NOs: 1-14 or 50-244, 248-249, 257-349, or any one or more of SEQ ID NOs: 1, 2, 15, 153, 304-349, or SEQ ID NO: 1 (and any of the above with a methionine at the N-terminus) or a fragment, homolog, or analog thereof.

For example, the present invention provides for a polynucleotide sequence comprising, consisting essentially of, or consisting of the following sequence: [SEQ ID NO: 251: ttt ctg ccc tca]; SEQ ID NO: 252: ttt ctg ccc tca gaa ttt gga gta gac gta gac aga] or other nucleic acid sequence that encodes a peptide of the invention including all nucleic acid sequences permitted under codon degeneracy which is the divergence in the genetic code which permits variation of nucleotide sequence without effecting the amino acid sequence of an encoded polypeptide. Accordingly, the instant invention relates to any nucleic acid sequence that encodes all or a substantial portion of the amino acid sequences set forth herein.

Preferably any of the nucleotide sequences herein are preferably isolated and/or purified.

The present invention also includes recombinant constructs comprising one or more of the nucleotide sequences described herein. Such constructs comprise a vector, such as a plasmid or viral vector, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. Appropriate cloning and expression vectors for use with prokaryotic and eukaryotic hosts are also described in Sambrook et al. (2001, Molecular Cloning: A Laboratory Manual, Cold Spring Harbor Press).

Examples of such expression vectors include chromosomal, nonchromosomal and synthetic DNA sequences, e.g., derivatives of or Simian virus 40 (SV40); bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host. The appropriate nucleic acid sequence may be inserted into the vector by a variety of procedures. In general a nucleic acid sequence encoding one of the polypeptides herein is inserted into an appropriate restriction endonuclease site(s) by procedures known in the art. Such procedures and related sub-cloning procedures are deemed to be within the scope of those skilled in the art.

The nucleic acid sequence in the expression vector is preferably operatively linked to an appropriate transcription control sequence (promoter) to direct mRNA synthesis. Examples of such promoters include: the retroviral long terminal (LTR) or SV40 promoter, the E. coli lac or trp promoter, the phage lambda PL promoter, and other promoters known to control expression of genes in prokaryotic or eukaryotic cells or their viruses. The expression vector also contains a ribosome binding site for translation initiation, and a transcription terminator. The vector may also include appropriate sequences for amplifying expression. In addition, the expression vectors preferably contain one or more selectable marker genes to provide a phenotypic trait for selection of transformed host cells such as dihydrofolate reductase or neomycin resistance for eukaryotic cell cultures, or such as tetracycline or ampicillin resistance in E. coli.

The vector containing the appropriate nucleic acid sequences as described above, as well as an appropriate promoter or control sequence, may be employed to transform an appropriate host to permit the host to express the peptides herein (e.g., SEQ ID NOs: 1-256 or SEQ ID NOs: 1-24, 50-244, and 248-249, 257-305, or SEQ ID NO: 1 or 2 or 153 or 304, or 305). Such vectors can be used in gene therapy.

Examples of appropriate expression hosts include: bacterial cells, such as E. coli, Streptomyces, Salmonella typhimurium; fungal cells, such as yeast; insect cells such as Drosophila and Spodoptera frugiperda (Sf9); animal cells such as CHO, COS, HEK 293 or Bowes melanoma; adenoviruses; plant cells, etc. The selection of an appropriate host is deemed to be within the scope of those skilled in the art from the teachings herein. The invention is not limited by the host cells employed.

Thus, in some embodiment, the present invention relates to methods for producing an analgesic peptide by transfecting a host cell (e.g., human cell or plant cell) with an expression vector comprising a nucleotide sequence that encodes a peptide comprising, consisting essentially of, or consisting of an amino acid sequence of (or encoded by) any one or more of SEQ ID NOs: 1-349 or any one or more of SEQ ID NOS: 1-244, 248-249, 257-349, or any of the above described polypeptides (e.g., any of the above with a methionine at the N-terminal, or any analgesic fragment, or any antagonist, or any anti-pyretic thereof, or any homolog or analog thereof or multiple copies of any of the above polypeptides on a single vector). Such host cells are then cultured under a suitable condition, which allows for the expression of such peptides.

The present invention further contemplates gene therapy using nucleic acids encoding one or more of the polypeptides herein (e.g., SEQ ID NOs: 1-349, or SEQ ID NOs: 1-24, 50-244, and 248-249, 257-349, or SEQ ID NOs: 1, 2, 153 or 304-349) or an analog or homolog thereof. Preferably, such gene therapy is targeted. Targeted gene therapy involves the use of vectors (organ- and tumor-homing peptides) that are targeted to specific organs or tissues after systemic administration. The vector consisted of a covalent conjugate of avidin and a monoclonal antibody to a receptor. For example, for delivery to the brain, a chimeric peptide had been monobiotinylated, to a drug transport vector. The vector consisted of a covalent conjugate of avidin and the OX26 monoclonal antibody to the transferrin receptor. Owing to the high concentration of transferrin receptors on brain capillary OX26 targets brain and undergoes receptor-mediated transcytosis through the blood-brain barrier (Bickel et al., 1993, Proc. Nat. Acad. Sci. 90:2618-2622). An other example is vector-mediated delivery of opioid peptides to the brain (NIDA Res Monogr. 1995, 154:28-46).

In some embodiments, such nucleic acids are used to create a transgenic plant or animal, wherein the transgenic plant or animal is transgenic for a polynucleotide of the present invention and expresses a polypeptide of the present invention. For introducing a nucleic acid encoding one or more of the peptides herein into a plant cell, introduction can be carried out by conventional gene engineering techniques, for example, Agrobacterium infection, electroporation into protoplasts, particle gun methods, and the like. In some embodiments, the nucleic acids above are introduced along with a second nucleic acid sequence or gene. In some embodiments, the second nucleic acid sequence can act as a promoter, etc.

Preferably, the nucleic acid that is introduced into a plant cell is integrated into a vector having a selection marker gene. For example, the nucleic acids encoding any one or more of SEQ ID NOs: 1-349 or any one or more of SEQ ID NOs: 1-24, 50-244, 248-249, or 257-349, or any one or more of SEQ ID NOs: 1, 2, 153, or 304-349, or a homolog or analog thereof can be integrated into one of such vectors (e.g., pGEM-T and pBIN binary vectors). The vectors are then introduced into a chromosome of a plant cell by homologous recombination (Fraley et al., 1983, Proc. Natl. Acad. Sci. USA, 80; 4803). Plant cells expressing the nucleic acids can then be selected. Alternatively, the nucleic acids can be introduced into a plant cell in a vector that it is operably linked to a promoter and optionally a terminator both of which can function in the plant cell.

Non-limiting examples of promoters that can function in a plant cell include constitutive promoters derived from T-DNA such as nopaline synthase gene promoter, octopine synthase gene promoter, etc., promoters derived from plant viruses such as 19 S and 35 S promoters derived from cauliflower mosaic virus, etc., inductive promoters such as phenylalanine ammonia-lyase gene promoter, chalcone synthase gene promoter, pathogenesis-related polypeptide gene promoter, etc., and the like.

Non-limiting examples of terminators that can function in a plant cell include terminators derived from T-DNA such as nopaline synthase terminator, terminators derived from plant viruses such as terminators derived from garlic viruses GV1, GV2, and the like.

Plant cells into which such nucleic acids are introduced include plant tissues, whole plants, cultured cells, seeds and the like. Examples of the plant species into which the genes are introduced include dicotyledones such as tobacco, cotton, rapeseed, sugar beet, Arabidopsis thaliana, canola, flax, sunflower, potato, alfalfa, lettuce, banana, soybean, pea, legume, pine, poplar, apple, grape, citrus fruits, nuts, etc.; and monocotyledones such as corn, rice, wheat, barley, rye, oat, sorghum, sugar cane, lawn, etc. The second gene may also be introduced into such plant cells.

The transformant plant cells expressing one or more of the polypeptides herein or homologs thereof can be obtained by culturing cells into which the gene is transferred in a selection culture medium corresponding to a selection marker joined to the locus on the gene, for example, a culture medium containing a cell growth inhibitor, or the like, and isolating a clone capable of growing in the culture medium. Further, the selection culture medium should also correspond to a selection marker joined to the locus of the second gene when the altered form of enzymatic activity is also present in the transformant plant cells. Alternatively, the above transformant plant cells can be selected by culturing plant cells into which the gene is introduced in a culture medium containing the weed control compound to which the resistance is given, and isolating clones capable of growing in the culture medium.

The plant expressing the desired peptide can be obtained from the transformant cells thus obtained by regenerating the whole plant according to a conventional plant cell culture method, for example, that described in Plant Gene Manipulation Manual, Method for Producing Transgenic Plants, 1996, UCHIMIYA, Kodansha Scientific). Thus, the transformed plants such as plant tissues, whole plants, cultured cells, seeds and the like can be obtained.

For example, rice and Arabidopsis thaliana expressing a gene encoding a desired peptide having the characteristics of having (i) anti-pyrogenic, (ii) anti-inflammatory, (iii) anti-neoplastic activity, or (iv) expressing resistance against pathogen, or (v) expressing developmental changes such as an increase in the number of flowers (e.g., any one or more of SEQ ID NOs: 1-349 or any one or more of SEQ ID NOs: 1-24, 50-244, and 248-249, 257-349, or any one or more of SEQ ID NOs: 1-2, 153, or 304-349, or a homolog or analog thereof) can be obtained according to the method described in Experimental Protocol of Model Plants, Rice and Mouse-Ear Cress Edition, Chapter 4 (1996, Supervisors: Shimamoto and Okada, Shujun-sha), Further, according to the method, soybean expressing a gene encoding a desired peptide by introducing the gene into soybean adventitious embryo with a particle gun. Likewise, according to the method described by Fromm et al., 1990, Bio/Technol., 8:838, corn expressing the gene can be obtained by introducing the gene into adventitious embryo with a particle gun. Wheat expressing the gene by introducing the gene into sterile-cultured wheat immature scutellum with a particle gun according to a conventional method described by Takumi, 1995, J. Breeding Soc., 44: Extra, 1:57. Likewise, according to a conventional method described by Hagio et al., 1995, J. Breeding Soc., 44; Extra, 1:67, barley expressing the gene encoding the above polypeptide can be obtained by introducing the gene into sterile-cultured barley immature scutellum with a particle gun.

An other embodiment is directed to fragments of the correspondent nucleic acid sequences, or the complement thereof, which may find use as, for example, hybridization probes or as antisense oligonucleotides. Such nucleic acid fragments are usually at least about 10 nucleotides in length, or at least about 20 nucleotides in length, or at least about 30 nucleotides in length, at least about 40 nucleotides in length, yet at least about 50 nucleotides in length, yet more, wherein in this context the term “about” means the referenced nucleotide sequence length plus or minus 10% of that referenced length.

In some embodiments, the present invention relates to methods for isolating a gene or gene fragment encoding a peptide of the invention (any one or more of SEQ ID NOs: 1-349 or a homolog or analog thereof) and homologs or analogs thereof from various organisms. Such gene or gene fragment can have (i) anti-pyrogenic, (ii) anti-inflammatory, (iii) anti-neoplastic activity, or (iv) expressing resistance against pathogen, or (v) expressing developmental changes such as an increase in the number of flowers. Such gene or gene fragment can be identified by performing PCR using genomic DNA or cDNA of an organism having the desired gene as a template and primers produced on the basis of nucleotide sequences corresponding to those about the N- and C-termini of the polypeptide to amplify the desired gene. Further, genes encoding a peptide can be obtained different organisms (e.g., a clone, a plant, an animal, etc.). For example, first, a cDNA library is constructed by obtaining mRNA from an organism and synthesizing cDNA by using the mRNA as template with reverse transcriptase and integrating the cDNA into a phage vector such as ZAP II, etc. or a plasmid vector such as pUC, etc. The cDNA library may be introduced into Escherichia coli followed by subjecting a complementation test to select clones containing the desired gene derived from the desired organism. Further, for amplifying a DNA fragment containing at least a part of the desired gene, PCR can be carried out by using the above-constructed cDNA library as a template and primers designed and synthesized on the basis of nucleotide sequences of the peptide. Screening of the cDNA library can be carried out by using the DNA fragment thus obtained as a probe to select positive clones. The desired gene, i.e., a gene encoding a peptide substantially having at least one characteristics of (i) to, (v), can be confirmed by determination of the nucleotide sequence of the selected clone.

3. Antibodies

In another embodiment, the invention provides for antibodies that specifically bind to any of the polypeptides herein. For example, the present invention contemplates an antibody that specifically binds to a peptide of having an amino acid sequence comprising, consisting essentially of, or consisting of any one or more of SEQ ID NOs: 1-349 or any one or more of SEQ ID NO: 1-244, 248-249, and 257-349, or any one or more of SEQ ID NOs: 1-14, 50-244, and 257-349, or any one or more of SEQ ID NOs: 1-2, 153, 304-349 or SEQ ID NO: 1, or any of the above with a methionine at the N-terminus, or any fragment, homolog, or analog thereof. The term “antibodies” is meant to include polyclonal antibodies, monoclonal antibodies, fragments thereof such as F(ab′)₂, and Fab fragments, as well as any recombinantly produced binding-partners.

Antibody can be prepared by conventional methods, e.g. by immunization of a human or of an animal, such as, for example, mouse, rat, guinea pig, rabbit, horse, sheep, goat, chicken (see also Messerschmid, 1996, BIOforum, 11:500-502), and subsequent isolation of the antiserum; or by establishing hybridoma cells and subsequent purification of the secreted antibodies; or by cloning and expression of the nucleotide sequences, or modified versions thereof, which encode the amino acid sequences which are responsible for the binding of the natural antibody to the antigen and/or hapten. Antibodies of the invention are in particular those antibodies which bind to a polypeptide comprising, consisting essentially of, or consisting of any one or more of: SEQ ID NOs: 1-349, or any one or more of SEQ ID NOs: 1-24, 50-244, 248-249, and 257-349, or any one or more of SEQ ID NOs: 1-24, 50-163, 248-249, and 257-349, or any one or more of SEQ ID NOs: 1-2, 153, 304-349 or SEQ ID NO: 1. In some embodiments, such polypeptide has less than 100 amino acid residues, 90, 80, 70, 60, 50, 40, 30, 20, 10, 9, 8, 7, 6, 5, or 4 amino acid residues. In some embodiments, such polypeptide has between 4-100, 4-50, 4-40, 4-30, or 4-20 amino acid residues. In some cases, such antibody selectively binds to the amino acid sequence Phe-Leu-Pro-Ser-Glu-Phe-Gly-Val-Asp-Val-Asp-Arg (SEQ ID NO: 2) or amino acid sequence Phe-Leu-Pro-Ser (SEQ ID NO: 1).

The antibodies herein can specifically bind with a Kₐ of greater than or equal to about 10⁴M⁻¹, or about 10⁵M⁻¹, or about 10⁶M⁻¹ or about 10⁷M⁻¹. Affinities of binding-partners or antibodies can be readily determined using conventional techniques, for example those described by Scatchard et al., 1949 (Ann. N.Y. Acad. Sci. 51:660) or, by surface plasmon resonance described by Wolff et al., 1993 (Cancer Res. 53:2560; BIAcore/Biosensor, Piscataway, N.J.), which are incorporated herein by reference for all purposes.

Polyclonal antibodies can be readily generated from a variety of sources, for example, horses, cows, goats, sheep, dogs, chickens, rabbits, mice or rats, using procedures that are well known in the art. In general, an isolated polypeptide of the invention (e.g., a polypeptide comprising, consisting essentially of, or consisting of an amino acid sequence of (or encoded by) any one or more of SEQ ID NOs: 1-349 or any of the above with an N-terminal methionine) that is appropriately conjugated, is administered to the host animal typically through parenteral injection. The immunogenicity of the polypeptide may be enhanced through the use of an adjuvant, for example, Freund's complete or incomplete adjuvant. Following booster immunizations, small samples of serum are collected and tested for reactivity to the polypeptide. Examples of various assays useful for such determination include those described in Antibodies. A Laboratory Manual, Harlow and Lane (eds.), Cold Spring Harbor Laboratory Press, 1988; as well as procedures such as countercurrent immuno-electrophoresis (CIEP), radioimmunoassay (RIA), radioimmunoprecipitation, enzyme-linked immunosorbent assays (ELISA), dot blot assays, and sandwich assays, see, e.g., U.S. Pat. Nos. 4,376,110 and 4,486,530, or other similar assays known in the art.

Monoclonal antibodies specific for a desired polypeptide antigen (such as the peptides described herein) may be readily prepared using well-known procedures, see for example, the procedures described in Current Protocols in Immunolog (Wiley & Sons, NY, Coligan et al., eds., 1994; U.S. Pat. Nos. RE 32,011, 4,902,614, 4,543,439 and 4,411,993; Monoclonal Antibodies, Hybridomas: A New Dimension in Biological Analyses, 1980, Plenum Press, Kennett et al., eds.). Briefly, the host animals, such as mice are injected intraperitoneally at least once, or at least twice at about three-week intervals with isolated and purified polypeptide herein (e.g., a polypeptide comprising, consisting essentially of, or consisting of an amino acid sequence of (or encoded by) any one or more of SEQ ID NOs: 1-349, or, conjugated polypeptide herein, optionally in the presence of adjuvant. Mouse sera are then assayed by conventional dot blot technique or antibody capture (ABC) to determine which animal is most suitable as a source of splenocytes for fusion to a myeloma partner cell-line. Approximately 2-3 weeks later, the mice are given an intravenous boost of the polypeptide. Mice are later sacrificed and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, PEG. The cell suspension containing fused cells is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as, ¹²⁵I-conjugated polypeptide (e.g., a polypeptide comprising, consisting essentially of, or consisting an amino acid sequence of (or encoded by) any one or more of SEQ ID NOs: 1-349, or any of the above with an N-terminal methionine) is added to each well followed by incubation. Positive wells can be subsequently detected by autoradiography. Positive clones can be grown in bulk culture and supernatants are subsequently purified over a Polypeptide A column (Pharmacia).

The monoclonal antibodies of the invention can be produced using alternative techniques, such as those described by Alting-Mees et al., 1990 (Monoclonal Antibody Expression Libraries: A Rapid Alternative to Hybridomas, Strategies, Mol. Biol. 3:1-2469). Similarly, binding partners can be constructed using recombinant DNA techniques to incorporate the variable regions of a gene that encodes a specific binding antibody. Larrick et al. describe such technique in Biotechnol, 7:394, 1989.

Other types of antibodies may be produced using the information provided herein in conjunction with the state of knowledge in the art. For example, antibodies that have been engineered to contain elements of human antibodies that are capable of specifically binding any of the peptide or to a polypeptide containing the peptide sequence herein are also encompassed by the invention. An additional method for selecting antibodies that specifically bind to a polypeptide, peptide or fragment thereof is by phage display, e.g., Winter et al., 1994, Annu. Rev. Immunol. 12: 433; Burton et al., 1994, Adv. Immunol. 57:191. Human or murine immunoglobulin variable region gene combinatorial libraries may be created in phage vectors that can be screened to select Ig fragments (Fab, Fv, sFv, or multimers thereof) that bind specifically to a polypeptide, peptide, or fragment thereof. See, e.g., U.S. Pat. No. 5,223,409; Huse et al., 1989, Science:1275; Kang et al., 1991 Proc. Natl. Acad. Sci. USA 88:4363; Hoogenboom et al., 1992, J. Molec. Biol. 227:381; Schlebusch et al., 1997, Hybridoma 16:47, and references cited therein. For example, a library containing a plurality of polynucleotide sequences encoding Ig variable region fragments may be inserted into the genome of a filamentous bacteriophage, such as M13 or a variant or analog thereof, in frame with the sequence encoding a phage coat polypeptide, for instance, gene III or gene VIII of M13, to create an M13 fusion polypeptide. A fusion polypeptide may be a fusion of the coat polypeptide with the light chain variable region domain and/or with the heavy chain variable region domain. Once isolated and purified, the antibodies may be used to detect the presence of a polypeptide, or a peptide of the present invention in a sample using established assay protocols. Further, the antibodies of the invention may be used therapeutically to bind to the peptides of the invention and alter their activity in vivo.

### Formulations

Any of the composition herein may be formulated into pharmaceutical, veterinary, cosmetic and/or agricultural formulations for administration to an organism.

Typically such formulations will include one or more acceptable carriers, excipients, or diluents. Pharmaceutically acceptable carriers for therapeutic use are well known in the pharmaceutical art, and are described, e.g., in Remington's Pharmaceutical Sciences, Gennaro, A R, ed., 20th edition, 2000: Williams and Wilkins Pa., USA. which is incorporate herein by reference for all purposes. Agriculturally acceptable carriers for therapeutic or prophylactic treatment of plants are also known in the art. Cosmetic and veterinary excipients are also known in the art.

For example, the compositions herein may be combined with one or more natural or synthetic, organic or inorganic material to facilitate their application into the plant. Such a carrier will generally be inert and acceptable in agriculture. Such carrier can be solid (e.g., clays, natural or synthetic silicates, silica, resins, waxes, or solid fertilizers) or liquid (e.g., water, alcohols, ketones, petroleum fractions, aromatic or paraffinic hydrocarbons, chlorinated hydrocarbons, or liquefied gases).

A pharmaceutical or agricultural formulation can also contain any kind of other compatible ingredients such as, for example, protective colloids, adhesives, thickening agents, thixotropic agents, penetrating agents, stabilizing agents, sequestering agents, fertilizers, anti-freeze agents, repellents, color additives, corrosion inhibitors, water-repelling agents, UV-stabilizers, pigments, dyes or polymers.

In some embodiments, the compositions herein may be formulated as a salt and be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5 that is combined with buffer prior to use. After pharmaceutically and physiologically acceptable compositions have been prepared, they can be placed in an appropriate container and labeled for treatment of an indicated condition.

While any suitable carrier known may be employed in a pharmaceutical formulation of this invention, the type of carrier will vary depending on the mode of administration and whether a sustained release is desired. Routes of delivery may include oral, inhaled, buccal, intranasal, and transdermal routes, as well as novel delivery systems such as the protective liposomes for oral delivery of peptides.

For agricultural uses, formulations can be in a liquid or spray or any other dry formulations.

For parenteral administration, such as subcutaneous injection, the carrier can include, e.g., any one or more of the following ingredients: water, saline, alcohol, a fat, a wax or a buffer.

For oral administration, a carrier can comprise of carbohydrate or polypeptide fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and polypeptides such as gelatin and collagen. If desired, disintegrating or solubilizing agents may be added, such as the cross-linked polyvinyl pyrrolidone, agar, alginic acid, or a salt thereof, such as sodium alginate. If desirable, the drug can be delivered in nanocapsules that would protect against proteolysis by proteases. Such carriers enable the compositions herein to be formulated as tablets, pills, dragees, capsules, liquids, gels, syrups, slurries, suspensions, and the like, for ingestion by the patient. Pharmaceutical preparations for oral use can be obtained through a combination of active compounds with solid excipient, suiting mixture is optionally grinding, and processing the mixture of granules, after adding suitable auxiliaries, if desired, to obtain tablets or dragee cores. As the composition may be peptide, such peptides can be put into a liposomal formulation to avoid degradation.

The pharmaceutical formulations herein are administration by intravenous injection or by local applications (e.g., topical or subdermal).

Formulations for topical administration can use a carrier that is a solution, emulsion, and ointment or gel base. The base, for example, may comprise one or more of the following: petrolatum, lanolin, PEGs, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109. In this regard, it is preferable that the microsphere be larger than approximately 25 μm.

Pharmaceutical compositions may also contain diluents such as buffers, antioxidants such as ascorbic acid, low molecular weight (less than about 10 residues) polypeptides, polypeptides, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. In some cases the compositions herein are formulated as a lyophilizate using appropriate excipient solutions (e.g., sucrose) as diluents.

Pharmaceutically acceptable formulations include compositions wherein the active ingredients (e.g., a polypeptide comprising of, consisting essentially of, or consisting of an amino acid sequence of (or encoded by) any one or more of SEQ ID NOs: 1-349, or any analog, or homolog thereof) are contained in an effective dose to achieve the intended purpose. The determination of an effective amount or dosage is well within the capability of those skilled in the art. Typically, an effective dose of a polypeptide of the present invention (e.g., a polypeptide comprising of, consisting essentially of, or consisting of an amino acid sequence of (or encoded by) any one or more of SEQ ID NOs: 1-349, or any analog or homolog thereof) for systemic administration is between about 0.001 μg to about 100 g, or between about 0.01 μg to about 50 g, or between about 0.1 μg to about 1 g, or between about 1 mg to about 500 mg per dose. For topical administration, the compositions herein may be delivered at dosage up to about 99, 95, 90, 80, 70, 60, 50, 40, or 30% w/w of the composition.

In some embodiments, the therapeutic effective dosages of SEQ ID NOs: 1-349 are the serum concentrations that in the range of 1-1000 mg/L, 5-500 mg/L, or 10-100 mg/L, or 10-20 mg/L or the active ingredient.

Any of the compositions herein may be co-formulated or co-administered with a second therapeutic agent. Examples of therapeutic agents include, but are not limited to, analgesic, antipyretic medicaments (fever reducers), anesthetics, anti-rheumatic agents, anti-inflammatory agents, antidepressants, anti-neoplastic agents, antimicrobial agents (e.g., antibiotics, antiviral agents, and antifungal agents), pesticides, herbicides, angiogenic agents, anti-angiogenic agents, inhibitors of neurotransmitters or neurotransmitters, any agent known to treat neurodegenerative conditions and wound healing, and combinations thereof.

The concentration of an active ingredient in the composition of the present invention, as applied to plants can be within the range of 0.01 to 30.0% by weight, especially 0.1 to 30% % by weight. In a primary composition, the amount of active ingredient can vary widely and can be, for example, from 5 to 95% by weight of the composition.

For any of the compositions herein, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans. Those of ordinary skill in the art are well able to extrapolate from one model (be it an in vitro or an in vivo model). A therapeutically effective dose refers to that amount of active ingredient, for example a polypeptide comprising of, consisting essentially of, or consisting of an amino acid sequence of (or encoded by) any one or more of SEQ ID NOs: 1-349 or any fragment, analog, or homolog thereof, which ameliorates the symptoms or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. Pharmaceutically and physiologically acceptable compositions, which exhibit large therapeutic indices, are preferred. The data obtained from cell culture assays and animal studies is used in formulating a range of dosage for human use. The dosage contained in such compositions can be within a range of circulating concentrations that include the ED₅₀ with little or no toxicity. The dosage varies within this range depending upon the dosage form employed, sensitivity of the patient, and the route of administration. The practitioner, in light of factors related to the subject that requires treatment, will determine the exact dosage. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutically and physiologically acceptable compositions maybe administered every 3 to 4 days, every week, or once every two weeks depending on half-life and clearance rate of the particular formulation.

Normal dosage amounts may vary from 0.001 μg to 100 g, up to a total dose of about 1 g, depending upon the route of administration. Guidance as to particular dosages and methods of delivery is provided in the literature and generally available to practitioners in the art. Those skilled in the art will employ different formulations for nucleotides than for polypeptides or their inhibitors. Similarly, delivery of polynucleotides or polypeptides will be specific to particular cells, cell types, organism being treated, conditions, locations, etc.

For example, for the prevention or treatment of pain, the appropriate dosage of an anti-pain medicament will depend on the type of condition to be treated, as defined above, the severity and course of the disease, whether the agent is administered for preventive or therapeutic purposes or, as a combination with other drugs, previous therapy, the patient's clinical history and response to the agent, and the discretion of the attending physician. The agent is suitably administered to the patient at one time or over a series of treatments.

Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” in Toxicokinetics and New Drug Development, Yacobi et al., eds., Pergamon Press, New York 1989, pp. 42-96. For example, depending on the type and severity of the disease, about 0.001 μg/kg to 1000 mg/kg of a therapeteuric agent (peptide or small molecule described herein) is administered to a patient.

For example, a daily dosage might range from about 0.1 mg/kg to 100 g/kg or more, depending on the factors mentioned above. In some cases, such as the administration of a composition comprising a peptide comprising Leu-Pro or Pro-Leu, an oral dose may be less than 100 mg/kg. Such dose can be repeated daily or at least 2, 3, 4, 5, 6, 7, 8, 9, or 10 times daily. Similar or greater dosages can be administered to a patient when a composition (pharmaceutical formulation) herein is administered topically. For example, a topical dose might range from 1 mg/m² to 100 g/m² and such dose can be applied topically daily or at least 2, 3, 4, 5, 6, 7, 8, 9, or 10 times daily. The compositions herein can also be administered subdermally or subquetaneoulsy at appropriate ranges such as those described herein for example.

For local administration or topical administration lower dosage may be required. For repeated administrations over several days or longer, depending on the condition, the treatment is sustained until a desired suppression of disease symptoms occurs. However, other dosage regimens may be useful. The progress of this therapy is easily monitored by conventional techniques and assays. Guidance as to particular dosages and methods of delivery is provided in the literature; see, for example, U.S. Pat. Nos. 4,657,760; 5,206,344; or U.S. Pat. No. 5,225,212. It is anticipated that different formulations will be effective for different treatment compounds and different disorders, that administration targeting one organ or tissue, for example, may necessitate delivery in a manner different from that to another organ or tissue.

The compositions may be administered in the form of a solid, liquid, gel or gas (aerosol). For example, for oral administeration, the composition (pharmaceutical formulation) can be delivered as a syrup, lozenger, pill, gel capsule, etc. For subdermal or subquetaneous delivery, it can be delivered in a liquid formulation. For topical administeration, the pharmaceutical composition can be delivered as a gel, cream or patch.

The present invention contemplates a pharmaceutical formulation comprising: a peptide comprising dipeptide Leu-Pro or conservative substitution dipeptide thereof, or a homolog or analog thereof, or a small molecule thereof, or a salt, metabolite, or prodrug thereof, or a nucleic acid encoding such peptide; and a pharmaceutical excipient.

The present invention also contemplates a pharmaceutical formulation comprising: a peptide comprising dipeptide Pro-Leu conservative substitution dipeptide thereof, or or a homolog, derivative, or analog thereof, or a small molecule thereof, or a salt, metabolite, or prodrug thereof or a nucleic acid encoding such peptide; and a pharmaceutical excipient.

The present invention also contemplates a pharmaceutical formulation comprising: a peptide comprising Xxx-Leu-Pro or a homolog or analog thereof, or a small molecule thereof, or a salt, metabolite, or prodrug thereof, or a nucleic acid encoding such peptide; and a pharmaceutical excipient. In some cases, Xxx is an aromatic amino acid or a derivative or analog thereof. For example, in some cases Xxx is Tyr or Phe or Trp or His or Pro or a conservative substitution of any of the above, or an analog or a derivative of any of the above.

The present invention also contemplates a pharmaceutical formulation comprising: a peptide comprising Xxx-Leu-Phe or a homolog or analog thereof, or a small molecule thereof, or a salt, metabolite, or prodrug thereof, or a nucleic acid encoding such peptide; and a pharmaceutical excipient. In some cases, Xxx is an aromatic amino acid or a derivative or analog thereof. For example, in some cases Xxx is Tyr or Phe or Trp or His or Pro or a conservative substitution of any of the above, or an analog or a derivative of any of the above.

The present invention also contemplates a pharmaceutical formulation comprising: a peptide comprising Leu-Pro-Xxx or a homolog or analog thereof, or a small molecule thereof, or a salt, metabolite, or prodrug thereof, or a nucleic acid encoding such peptide; and a pharmaceutical excipient. In some cases Xxx is an amino acid such as Tyr, Thr, Glu, Asp or Ser or a conservative substitution of any of the above, or an analog or a derivative of any of the above.

The present invention also contemplates a pharmaceutical formulation comprising: a peptide comprising Ser-Leu-Xxx or a homolog or analog thereof, or a small molecule thereof, or a salt, metabolite, or prodrug thereof, or a nucleic acid encoding such peptide; and a pharmaceutical excipient. In some cases, Xxx is an aromatic amino acid or a derivative or analog thereof. For example, in some cases Xxx is Tyr or Phe or Trp or His or Pro or a conservative substitution of any of the above, or an analog or a derivative of any of the above.

The present invention also contemplates a pharmaceutical formulation comprising: a peptide comprising Xxx-Leu-Pro-Xxx or a homolog or analog thereof, or a small molecule thereof, or a salt, metabolite, or prodrug thereof, or a nucleic acid encoding such peptide; and a pharmaceutical excipient. In some cases, each of Xxx can be Tyr or Phe or Trp or His or Pro or an analog or a derivative thereof. In some cases, each of Xxx can be Phe or Ser or a conservative substitution of any of the above, or an analog or a derivative of any of the above.

The present invention also contemplates a pharmaceutical formulation comprising: a peptide comprising Xxx-Pro-Leu-Xxx or a homolog or analog thereof, or a small molecule thereof, or a salt, metabolite, or prodrug thereof or a nucleic acid encoding such peptide; and a pharmaceutical excipient. In some cases, each of Xxx can be Tyr or Phe or Trp or His or Pro or an analog or a derivative thereof. In some cases, each of Xxx can be an aromatic amino acid or a derivative or analog thereof. For example, in some cases Xxx is Tyr or Phe or Trp or His or Pro or a conservative substitution of any of the above, or an analog or a derivative of any of the above. In some cases, Xxx is Ser or a conservative substitution of Ser, or an analog or a derivative of Ser.

The present invention also specifically contemplates pharmaceutical formulation comprising a composition comprising, consisting, or consisting essentially of tripeptides such as: Phe-Leu-Pro, Trp-Leu-Prp, Tyr-Leu-Pro, Pro-leu-Phe, Pro-Leu-Trp, and Pro-Leu-Tyr. Any of the above amino acids can be substituted by a conservative substitution, or an analog or derivative of any of the above. The invention also contemplates non-linear (e.g., cyclic) forms of the above and small molecules equivalent to any of the above.

Any of the excipients described herein or any other ones known in the art can be used according to the present invention.

The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred compositions contain, in addition to the compositions herein one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Injectable formulations of the compositions herein are preferably sterile. Means for achieving sterility are well known in the art.

For delivery to the dermis and/or epithelium, dermal patches and delivery systems, utilizing active or passive transdermal delivery carriers may be prepared suing well known methods and materials, including, for example, microporous membranes, silicon polymers and diffusion matrixes. Such materials and methods are described, for example, in: Remington's Pharmaceutical Sciences, supra.

For use in plants, the compositions of the invention are generally applied to seeds, plants or their habitat. Thus, the compositions herein can be applied directly to the soil before, at or after drilling so that the presence of active compound in the soil can control the growth of pathogens, which may attack the seeds. When the soil is treated directly with a composition herein, it can be applied in any manner which allows it to be intimately mixed with the soil, e.g., by spraying, by broadcasting a solid form of granules, or by applying the active ingredient at the same time as drilling by inserting it in the same drill as the seeds. A suitable application rate is within the range of from 0.005 to 1000 g per hectare, or from 0.10 to 500 g per hectare.

Alternatively, the active compounds can be applied directly to a plant by, for example, spraying or dusting either at the time when a pathogen has begun to appear on the plant or before the appearance of a pathogen as a protective measure. In both such cases the preferred mode of application is by foliar spraying. It is generally important to obtain good control of pathogens in the early stages of plant growth, as this is the time when the plant can be most severely damaged.

The spray or dust can further contain a pre- or post-emergence herbicide if this is thought necessary. Sometimes, it is practicable to treat the roots, bulbs, tubers or other vegetative propagule of a plant before or during planting, for example, by dipping the roots in a suitable liquid or solid composition. When the active compound is applied directly to the plant a suitable rate of application is from 0.002 to 5 kg per hectare, or from 0.005 to 1 kg per hectare, or from 0.01 to 0.05 kg per hectare.

### Conditions Affecting Animals and Plants

In some aspects, the present invention relates to uses of compositions such as the peptides disclosed herein (Table VI) for modulating, preventing, or treating condition(s) in an organism. Such organisms can be animals and/or plants. Examples of animals include domesticated animals, such as dogs, cats, horses, cows, goats, sheep, chicken, and birds. Animals can also be humans. Plants can be crops such as wheat, barley, rice, corn, sugar, or soy; vegetables or fruits, such as apples, pears, citrus fruits, berries and nuts; and/or flowering plants such as roses, gardenias, orchids, carnations, bird of paradise, etc. But other plants or parts of plants are also contemplated herein (e.g., trees for lumber, such as fir, redwoods, pine, etc.).

The conditions that are modulated, prevented, or treated by the compositions herein can be broadly classified as metabolic or mitochondrial conditions. More specifically, such conditions are e.g., thermogenic or pyrogenic conditions. Such conditions can be associated with, for example, pain, temperature regulation, inflammation, neoplastic growth (e.g., cancer), innate immune response activation and ability to fight parasites and pathogens, skin and dermatological conditions, diabetes related disorders, wound healing, undesirable drag side effects, and neurological and neurodegenerative conditions.

Such conditions can occur in a cell, group of cells, or an entire organism to be treated herein.

1. Pain

In one aspect the present invention relates to treatment of pain. Examples of pain conditions contemplated by the invention include, but are not limited to, headaches (e.g., trigeminal neuralgia, sinusitis, cluster headaches, migraines, etc.), low back pain, cancer pain, arthritis pain, muscle spasm pain (muscle cramps), bone pain, pain resulting from burns, pain associated with bumps, pain associated with bruises, inflammatory pain (from an infection or arthritic disorder), pain from obstructions, myofascial pain, pain from Nerve trauma (dystrophy/causalgia), phantom limb pain, entrapment neuropathy (e.g., carpal tunnel syndrome), peripheral neuropathy, and pain from wounds, e.g., surgical, accidental, or self-inflicted wounds.

The pathophysiology of pain can be broadly divided into three categories: (i) nociceptive pain, (ii) neuropathic pain, and (iii) idiopathic pain. (Willis, W. D., 1985, The Pain System. The Neural Basis of Nociceptive Transmission in the Mammalian Nervous System. Pain and Headache, vol. 8, Gildenberg PL (Ed.) Karger Publishers, New York).

Nociceptive pain is the result of receptor stimulation by tissue injury. It involves the normal activation of the nociceptive system by noxious stimuli. Examples of nociceptive pain include sprains, bone fractures, burns, bumps, bruises, inflammation (from an infection or arthritic disorder), obstructions, myofascial pain (which may indicate abnormal muscle stresses) headaches, low back pain, cancer pain, and arthritis pain. In some embodiments, the compositions herein are used to prevent or treat nociceptive pain. Such compositions include, e.g., those that comprise a peptide comprising any one or more of SEQ ID NOs: 1-349, or SEQ ID NO: 1-244, 248-249, and 257-349 or: SEQ ID NO: 1, 2, 153, 304-349, or SEQ ID Nos: 1. Such composition can comprise a nucleic acid encoding an amino acid sequence of any one or more of SEQ ID NOs: 1-349, or any one or more of SEQ ID NO: 1-244, 248-249, and 257-349 or any one or more of: 1, 2, 153, or 304-349. Such compositions can also include a small molecule that mimics a peptide comprising any one or more of: SEQ ID NOs: 1-349, or any one or more of SEQ ID NO: 1-244, 248-249, and 257-349 or any one or more of: SEQ ID NO: 1, 2, 153, 304-349, or SEQ ID NO: 1. In some embodiments, second therapeutic agent(s) such as NSAIDs (Non-Steroidal Anti-Inflammatory Drugs), and/or opioids, and/or antagonists can be used in combination with the compositions herein to treat nociceptive pain.

Thus, in one aspect, the present invention relates to uses of the compounds herein for treating nociceptive pain. Such methods involve administering one or more of the compositions herein to a subject suffering or susceptible of suffering nociceptive pain. Such composition can include, e.g., a polypeptide comprising, consisting essentially of, or consisting of an amino acid sequence of any one or more of SEQ ID NOs: 1-349, or any one or more of SEQ ID NOs: 1-14, 50-152, 248-249, and 257-349, or any one or more of SEQ ID NOs: 1-2, 153, 304-349 or SEQ ID NO: 1. In one embodiment, such composition comprises a nucleic acid sequence that encodes a polypeptide comprising, consisting essentially of, or consisting of an amino acid sequence of any one or more of SEQ ID NOs: 1-349, or any one or more of SEQ ID NOs: 1-14, 50-152, 248-249, and 257-349, or any one or more of SEQ ID NOs: 1-2, 153, 304-349 or SEQ ID NO: 1. In one embodiment, such composition comprises an antibody that specifically binds a polypeptide comprising, consisting essentially of, or consisting of an amino acid sequence of any one or more of SEQ ID NOs: 1-349, or any one or more of SEQ ID NOs: 1-14, 50-152, 248-249, and 257-349, or any one or more of SEQ ID NOs: 1-2, 153, 304-349 or SEQ ID NO: 1. In some embodiment, the composition comprises a small molecule that mimics a polypeptide of any one or more of SEQ ID NO: 1-349, or any one or more of SEQ ID NOs: 1-14, 50-152, 248-249, and 257-349, or any one or more of SEQ ID NOs: 1-2, 153, 304-349 or SEQ ID NO: 1.

The second category of pain, neuropathic pain, is the result of an injury or malfunction in the peripheral or central nervous system. Examples of neuropathic pain include post herpetic (or post-shingles) neuralgia, reflex sympathetic dystrophy/causalgia (nerve trauma), components of cancer pain, phantom limb pain, entrapment neuropathy (e.g. carpal tunnel syndrome), and peripheral neuropathy most commonly caused by diabetes or chronic alcohol use.

Neuropathic pain is often triggered by an injury, but this injury may or may not involve actual damage to the nervous system. For example, nerves can be infiltrated or compressed by tumors, strangulated by scar tissue, or inflamed by infection, which may cause neuropathic pain. Neuropathic pain may persist for months or years beyond the apparent healing of any damaged tissues. Therefore, neuropathic pain is frequently chronic, not fully reversible, and tends to have a less robust response to treatment with opioids, but may respond to drugs such as anticonvulsants (carbamazepine and valproic acid, and gabapentin) and neuromodulating drugs (including tricyclic antidepressants, such as amitriptyline, imipramine, and desipramine).

The present invention contemplates uses of the compositions herein for treatment of neuropathic pain. In particular, the present invention includes methods for treating neuropathic pain in a subject by administering one or more of the compositions herein to the subject in a therapeutically effective amount to treat or prevent neuropathic pain. In preferred embodiment, the composition herein used to treat neuropathic pain comprises or consists essentially or consists of a polypeptide comprising, consisting essentially of, or consisting of an amino acid sequence such as any one or more of SEQ ID NO: 1-349, or an analog, salt, polymorph, metabolite, or prodrug thereof. In one embodiment, such composition comprises a nucleic acid sequence that encodes a polypeptide comprising, consisting essentially of, or consisting of an amino acid sequence such as any one or more of SEQ ID NO: 1-349, or a homolog or an analog thereof. In one embodiment, such composition comprises an antibody that specifically binds a polypeptide comprising, consisting essentially of, or consisting of an amino acid sequence such as any one or more of SEQ ID NO: 1-349 or an analog or homolog thereof.

The third category of pain, idiopathic pain, is a diagnosis of exclusion in which a patient suffers pain for longer than 6 months for which there is no physical cause and no specific mental disorder. Examples of idiopathic pain include, but are not limited to, arthritis, fibromyalgia, chronic fatigue syndrome, irritable bowel syndrome, interstitial cystitis, vulvadynia, carpal tunnel syndrome, etc.

In one aspect, the present invention relates to uses of the compounds herein for treating idiopathic pain. Such methods involve administering one or more of the compositions herein to a subject suffering or susceptible of suffering idiopathic pain. Such composition preferably include a polypeptide comprising, consisting essentially of, or consisting of an amino acid sequence such as any one or more of SEQ ID NO: 1-349, or an analog, salt, polymorph, metabolite, or prodrug thereof. In one embodiment, such composition comprises a nucleic acid sequence that encodes a polypeptide comprising, consisting essentially of, or consisting of an amino acid sequence such as any one or more of SEQ ID NO: 1-349, or a homolog or an analog thereof. In one embodiment, such composition comprises an antibody that specifically binds a polypeptide comprising, consisting essentially of, or consisting of an amino acid sequence such as any one or more of SEQ ID NO: 1-349 or an analog or homolog thereof.

Any of the compositions herein can be administered either singly or in combination with a second therapeutic agent such as an analgesic pain reliever or anti-inflammatory. In some embodiments, the second therapeutic agent is co-formulated with one or more of the compositions (e.g., polypeptides or analogs, i.e., small molecules equivalent thereof) herein.

According to the National Drug Classification (NDC), analgesics can be categorized in to the following group: general analgesic, narcotic analgesic, non-narcotic analgesic, anti-arthritics, anti-migraine/headache, central pain syndrome, NSAID, anti-pyretic, and anti-menstrual pain products. These categories can be combined into broader categories of analgesics entitled: narcotic analgesics, non-narcotic analgesics, and NSAIDs.

The present invention relates to a pharmaceutical formulation comprising the combination of the compositions herein (e.g., polypeptides, small molecules equivalent thereof, nucleic acids, antibodies) with one or more analgesic agents selected from the group consisting of general analgesic, narcotic analgesic, non-narcotic analgesic, anti-arthritics, anti-migraine/headache, central pain syndrome, NSAID, anti-pyretic, and anti-menstrual pain products. The present invention also relates to a pharmaceutical formulation comprising the combination of the polypeptides herein with one or more analgesics selected from the group consisting of narcotic analgesics, non-narcotic analgesics, and NSAIDs. The present invention also relates to methods of treating a subject suffering from pain (e.g., nociceptive pain, neuropathic pain, and idiopathic pain) comprising administered to the subject the one or more compositions herein and the one or more analgesics described herein (either separately or in combination, as a co-formulation or in two separate formulations).

Examples of narcotic analgesics include, but are not limited to, Alfentanil; Allylprodine; Alphaprodine; Amiphenazole, Anileridine, Benzoylhydrazone, Benzylmorphine, Benzitramide, Nor-Binaltorphimine, Bremazocine; Bupremorphine; Butorphanol (Stadol); Clonitazene; Codeine; CTOP; Cyclazocine; DAMGO; Desomorphine; Dextromoramide; Dezocine; Diampromide; Dihydrocodeine; Dihydrocodeine enol acetate; Dihydromorphine; Dimenoxadol; Dimepheptanol; Dimethylthiambutene; Dioxaphetyl Butyrate; Dipipanone; Diprenorphine; DPDPE; Eptazocine; Ethoheptazine; Ethylketocyclazocine; Ethylmethylthiambutene; Etonitazene; Etorphine; Fentanyl (Sublimaze, Duragesic); Hydrocodone; Hydromorphone (Dilaudid); Hydroxypethidine; Isomethadone; ketobemidone; Levorphanol; Levallorphan; Lofentanil; Loperamide; Meperidine (Demerol); Meptazinol; Metazocaine; Methadone (Dolophine); Metopon; Morphine (Roxanol); Myrophine; Nalbuphine; Nalmefene; Nalorphine; Naloxone; Naltrindole; Naltrexone; Narceine; Nicomorphine, Norlevorphanol; Normethadone; Normorphine; Norpipanone; Opium; Oxycodone (OxyContin); Oxymorphone; Papaveretum; Papaverine; Pentazocine; Phenadoxone; Phenazocine; Phenoperidine; Piminodine; Pirtramide; Proheptazine; Promedol; Propiram; Propoxyphene (Darvon); Remifentanil; Spiradoline; Sufentanil; Tilidine; U50,488; and U69,593. Some products are combination drugs; codeine/acetaminophen (APAP; Tylenol #3); hydrocodone/acetaminophen (Vicodin); Oxycodone/ASA (Percodan); oxycodone/APAP (Percocet); propoxyphene/ASA (Darvon Compound); propoxyphene/napsylate (Darvocet-N); hydrocodone/ibuprofen (Vicoprofen); pentazocine/naloxone (Talwin-Nx).

Examples of non-narcotic analgesics, include, but are not limited to, Acetaminophen (Paracetamol; Tylenol); aspirin (acetylsalicylic acid; Anacin, Ascriptin, Bayer, Bufferin, Ecotrin, Excedrin); Aminobenzoic Acid; Capsaicin (Zostrix and Zostrix-HP); Carbaspirin Calcium; Choline and Magnesium Salicylates (CMT, Tricosal, Trilisate); Choline Salicylate (Arthropan); Etanercept; Fluprednisolone; Gold sodium Thiomalate; Gold Sodium Thiosulfate; Hyaluronic Acid; Homomethyl Salicylate; Leflunomide; Magnesium Salicylate (Arthritab, Bayer Select, Doan's Pills, Magan, Mobidin, Mobogesic); Menthol; Methorexate; Octyl Salicylate; Oxyphenbutazone; Phenyl Salicylate; Phenylbutazone; Prednisolone; Salicylamide; Salsalate (Amigesic, Anaflex 750, Disalcid, Marthritic; Mono-Gesic, Salflex, Salsitab); Sodium Hyaluronate; Sodium Salicylate; o-Acetylsalicyloyl Chloride; Sodium Thiosalicylate (Thiocyl); Tramadol; Triamcinilone; Triethanolamine Salicylate (Trolamine); Zomepirac. Some products, such as Excedrin, are combination drugs (Excedrin is acetaminophen, ASA, and caffeine). Other non-narcotic gabapentin (Neurontin); lamotrigine and, anti-convulsants and tricyclic anti-depressants such as carbamazepine, pregabalin and duloxetine.

Examples of NSAIDS include, but are not limited to, Bromfenac Sodium; Celecoxib (Celebrex); Diclofenac Potassium (Cataflam); Diclofenac Sodium (Voltaren, Voltaren XR); Diclofenac Sodium with misoprostol (Arthrotec); Diflunisal (Dolobid); Etodolac (Lodine, Lodine XL); Etadolac; Fenoprofen calcium (Nalfon); Flurbiprofen (Ansaid); Ibuprofen (Motrin, Advil, Nuprin); Indomethacin (Indocin, Indocin SR); Ketoprofen (Actron, Orudis, Orudis KT, Oruvail); Meclofenamate Sodium (Meclomen); Mefenamic acid (Ponstel); Meloxicam (Mobic); Nabumetone (Relafen); Naproxen (Naprosyn, Naprelan, Alleve, Anaprox); Oxaprozin (Daypro); Piroxicam (Feldene); Piroxicam (Feldene); Rofecoxib (Vioxx); Sulindac (Clinoril); Suprofen; Tolmetin Sodium (Tolectin); Valdecoxib (Bextra).

Examples of antagonists include, but are not limited to, Alvimopan, trans-3,4-dimethyl-4-(3-hydroxyphenyl)piperidine; ANTI, 5′-acetamidinoethylnaltrindole; 4-Aminoquinoline; N-(4-amino-2-methylquinolin-6-yl)-2-(4-ethylphenoxymethyl)benzamide monohydrochloride; Benzimidazolinone; 7-Benzylidenenaltrexone; Binaltorphimine; nor-Binaltorphimine; Butorphanol (17-cyclobutylmethyl-3,14-dihydroxymorphinan) tartrate; CTAP, D-Phe-Cys-Tyr-D-Trp-Arg-Thr-Pen-Thr-NH2; CTOP, D-Phe-Cys-Tyr-D-Trp-Orn-Thr-Pen-Thr-NH2; Cyclazocine; Cyprodime; 1,3-Dimethyl-4-piperidinone; Ethylketocyclazocine; β-Funaltrexamine; GNTI, 5′-Guanidinonaltrindole; ICI 174864, N,N-diallyl-Tyr-Aib-Aib-Phe-Leu; Indolomorphinan; 5¢-Isothiocyanate; J-113397, 1-[(3R,4R)-1-Cyclooctylmethyl-3-hydroxymethyl-4-piperidyl]-3-ethyl-1,3-dihydro-2H-benzimidazol-2-one; JDTic, (3R)-7-Hydroxy-N-[(1S)-1-[[(3R,4R)-4-(3-hydroxyphenyl)-3,4-dimethyl-1-piperidinyl]methyl]-2-methylpropyl]-1,2,3,4 tetrahydro-3-isoquinoline-carboxamide 3-Quadazocine; Loperamide; Methoxynaltrexone; Methylnaltrexone; Mr 2266; Nalmefene; Nalorphine; Naloxone; Naloxone methiodide; Naloxazone; Naltrexone; β-Naltrexamine; Naltriben; Naltrindole; Phenylpiperidine; SB-612111, (-)-cis-1-methyl-7-[[4-(2,6-dichlorophenyl)piperidin-1-yl]methyl]-6,7,8,9-tetrahydro-5H-benzocyclohepten-5-ol; SoRI 9409, 59-(4-chlorophenyl)-17-(cyclopropylmethyl)-6,7-didehydro-3,14-dihydroxy-4,5a-epoxypyrido-[29,39:6,7]morphinan, SNC 80; TIPP-y, Tyr-Tic-Phe-Phe; Triethyleneglycolnaltrexamine.

Furthermore, there are various naturally occurring and synthetic opioids that can be used to treat pain. See Table II below.

TABLE II
List of opioid peptides

| | |
|---|---|
| Enkephalins | |
| [Leu]-enkephalin | YGGFL |
| | (SEQ ID NO: 15) |
| [Met]-enkephalin | YGGFM |
| | (SEQ ID NO: 16) |
| Rimorphin | YGGFLRRQFKVVT |
| | (SEQ ID NO: 248) |
| Leumorphin | KYPKRSSEVAGEGDGD |
| | SMGHEDLYKRYGGFLR |
| | RIRPKLKWDNQKRYGG |
| | FLRRQFKVVTRSQEDP |
| | NAYSGELFDA |
| | (SEQ ID NO: 249) |
| Endorphins | |
| α-Neoendorphin | YGGFLRKYPK |
| | (SEQ ID NO: 17) |
| β-Neoendorphin | YGGPLRKYP |
| | (SEQ ID NO: 18) |
| β-human-Endorphin | YGGFMTSEKSQTPLVTLFKN |
| | AIIKNAYKKGE |
| | (SEQ ID NO: 19) |
| α-human-Endorphin | YGGFMTSEKSQTPLVT |
| | (SEQ ID NO: 20) |
| Dynorphins | |
| DynorphinA | YGGFLRRIRPKLKWDNQ |
| | (SEQ ID NO: 21) |
| Dynorphin B | YGGFLRRQFKVVT |
| | (SEQ ID NO: 22) |
| Endomorphins | |
| Endomorphin-1 | YPTF |
| | (SEQ ID NO: 23) |
| Endomorphin-2 | YPFF |
| | (SEQ ID NO: 24) |
| Synthetic peptides | |
| [D-Ala2, N-Me-Phe4, | [Tyr-D-Ala-Gly-N- |
| Gly5-ol]-enkephalin | Methyl-Phe-Gly-ol |
| (DAMGO; DAGO) | (SEQ ID NO: 25) |
| D-Pen2,5]-enkephalin | [Tyr-D-Pen-Gly-Phe- |
| (DPDPE) | D-Pen |
| | (SEQ ID NO: 26) |
| D-Pen2,5]-enkephalin | Tyr-D-Pen-Gly-D- |
| (pCl-DPDPE) | Chloro-Phe-D-Pen |
| | (SEQ ID NO: 27) |
| [D-Pen2, Pen5]-enkephalin | Tyr-D-Pen-Gly-Pen- |
| (DPLPE) | Pen |
| | (SEQ ID NO: 28) |
| [D-Ser2, D-Leu5]-enkephalin- | Tyr-D-Ser-Gly-Phe- |
| Thr (DSLET) | Leu-Thr |
| | (SEQ ID NO: 29) |
| [D-Ala2, D-Leu5]-enkephalin | Tyr-D-Ala-Gly-Phe-D- |
| (DADLE) | Leu |
| | (SEQ ID NO: 30) |
| Met-enkephalin-Arg-Phe (MERF) | Tyr-Gly-Gly-Phe-Met- |
| | Arg-Phe |
| | (SEQ ID NO: 31) |
| CTOP | D-Phe-Cys-Tyr-D-Trp- |
| | Orn-Thr-Pen-Thr |
| | (SEQ ID NO: 32) |
| Ac-RYYRIK | Ac-Arg-Tyr-Arg-Ile- |
| | Lys |
| | (SEQ ID NO: 33) |
| ([D-Arg2, Lys4]-Dermorphin1)- | Tyr-D-Arg-Phe-Lys |
| amide(DALDA) | (SEQ ID NO: 34) |
| (D-Ala2,N-Methyl-Phe4,Met | Tyr-D-Ala-Gly-N- |
| (O)5-ol]-enkephalin | Methyl-Phe-Gly-ol |
| (FK-33824) | (SEQ ID NO: 35) |
| [D-Ala2,Leu5,Cys6]- | Tyr-D-Ala-Gly-Phe-D- |
| enkephalin (DALCE) | Leu-D-Cys |
| | (SEQ ID NO: 36) |
| [D-Ala2 Glu4]-Deltorphin II | Tyr-D-Ala-Phe-Glu- |
| | Val-Val-Pro Gly- |
| | amide |
| | (SEQ ID NO: 37) |
| [D-Ala2]Deltorphin 1 | Tyr-D-Ala-Phe-Asp- |
| | Val-Val-Gly |
| | (SEQ ID NO: 38) |
| PL-017 | Tyr-Pro-Methyl-Phe- |
| | D-Pro |
| | (SEQ ID NO: 39) |
| ICI 174,8674 | N,N-diallyl-Tyr-Aib- |
| | Aib-Phe-Leu |
| | (SEQ ID NO: 40) |
| Others | |
| Morphiceptin | YPPP |
| | (SEQ ID NO: 41) |
| Nociceptin orphanin FQ | FGGFTGARKSARKLANQ |
| | (SEQ ID NO: 42) |
| Nocistatin | TEPGLEEVGEIEGQKQLQ |
| | (SEQ ID NO: 43) |
| Neuropeptide AF human (NPAF) | AGEGLNSQFWSLAAPQRF |
| | (SEQ ID NO: 44) |
| Neuropeptide SF human (NPSF) | SQAFLFQPQRIF |
| | (SEQ ID NO: 45) |
| Substrate P | RPKPQQFFGLM |
| | (SEQ ID NO: 46) |
| β-human-Casamorphin | YPPVEPIP |
| | (SEQ ID NO: 47) |
| β-bovine-Casomorphin | YPFPGPI |
| | (SEQ ID NO: 48) |

Of the above opioids β-endorphins, enkephalins, and dynorphin are three human endogenous opioids. β-Endorphins are primarily found in the arcuate nucleus of the hypothalamus and in the pituitary gland, a feature that distinguishes this group from the enkephalins, which are not present in that area. Enkephalins may be broken down into two types, methionoine enkephalin (met-enkephalin) and leucine enkephalin (leu-enkephalin), and their ratio is 4:1 respectively. They are more widely distributed in the brain than β-endorphins, being present in several areas including hypothalamic nuclei, limbic structures, caudate-putamen, the brain stem, several layers of the dorsal horn, peripheral nerves, and the adrenal medulla. The most powerful of the opioids, dynorphins, are found throughout the central and peripheral nervous systems. Some research supports the theory that they regulate pain at the spinal cord level, influence feeding behavior at the hypothalamic level, and function with other endogenous opioids to regulate the cardiovascular system. Dynorphins also may be involved in inhibiting intestinal motility, a phenomena that occurs when the body perceives pain. The presence of a large precursor to this opioid in the anterior pituitary suggests that it has many peripheral targets. An other opioid called neo-endorphin also is classified in the Dynorphin group.

The endogenous opioid system has been used to treat chronic pain through a technique called neuroaugmentation that involves electrical stimulation of specific areas of the brain to increase the quantity and reactivity of endogenous opioids. Partial or complete pain relief has been noted in patients treated with neuroaugmentation; lower levels of efficacy were observed in severely ill cancer patients. Spinal cord stimulation was found to be successful in treating chronic pain not associated with malignancy.

In some embodiments, the present invention contemplates the use of an opioid, e.g., any of the opioids herein (whether naturally occurring or not) to modulate heat production, or to modulate the innate immune mechanisms, or to modulate mitochondrial activity in plants and/or animals.

It should be noted that for the treatment/prevention/alleviation of pain, the compositions described herein can be used as analgesics as well as anesthetics.

2. Temperature Regulation

In one aspect, the compositions herein can also be used to regulate body temperature of an animal or a plant by modulating the outflow and inflow of heat from the body. Fever, an elevated core body temperature, is the most common thermoregulatory change that often accompanies inflammation and/or infection. One main source of heat in both plants and animals is the mitochondria. An other major source of heat in animals is muscular contraction. However, in plants, it is often difficult to measure their body/appendix temperature because the amount of heat produced is relatively small and the there is a large amount of heat loss to the environment.

In humans, opioids that have been used to alleviate pain have been linked to some degree with temperature regulation in humans. In particular, there is some evidence that suggests a link between the nervous system and thermoregulation (Thermoregulation, Tenth International Symposium on the Pharmacology of Thermoregulation. Blatteis C M, Ed., Annal. NY Acad. Sci. vol., 813, 1997), and more specifically between opioid peptides and thermoregulation (Adler M W et al, 1988, Opioid System and Temperature Regulation, Annu. Rev. Pharmacol. Toxicol., Vol. 28: 429-450).

In one aspect, the present invention relates to the surprising discovery that analgesics, such as opioids useful in treating pain in humans, can also be used to modulate heat production in plants. Thus, the present invention relates to uses of any of the compositions herein including for example (i) polypeptides comprising, consisting essentially of, or consisting of, any one or more of amino acid sequences SEQ ID NO: 1-349 or any one or more of amino acids sequences SEQ ID NOS: 50-244, 248-249, 257-349, or any one or more of amino acid sequences SEQ ID NO: 1-14, 50-244, 257-349, or any one or more of amino acid sequences SEQ ID NO: 1-2 or SEQ ID NO: 1 or an analog or homolog of any of the above; (ii) nucleic acids encoding any of the above polypeptides; (iii) antibody or antibody fragment that specifically bind any of the above polypeptides, or small molecules or nucleic acids; and (iv) small molecules or analogs of the above polypeptides.

Such heat production modulation can be used e.g., to prevent frost damage to the seed and plants. It can also increase the innate immune response of the plants (discussed in more detail below). The above uses can be accomplished by administering to a plant or seed any of the above compositions with an agricultural excipient via spray, drip irrigation or other irrigation, dipping at least a portion of said plant or seed in said composition, coating at least partially said plant or seed with said composition, etc. In another embodiment, a nucleic acid sequence encoding any of the above compositions can be used to transfect plants such that their heat production is regulated.

In another aspect, the present invention relates to methods of using the above compositions for modulating mitochondrial activity in plants and animals. Modulating mitochondrial activity can be used to prevent, treat, or ameliorate mitochondrial conditions in plants and animals.

Examples of mitochondrial conditions include, but are not limited to, Alpers disease (progressive infantile poliodystrophy); Barth syndrome (cardiomyopathy-neutropenia syndrome); lethal infantile cardiomyopathy (LIC); Beta-oxidation defects; carnitine deficiency and disorders; chronic progressive external opthalmoplegia syndrome (CPEO); Kearns-Sayre syndrome (KSS); lactic acidosis; Leber hereditary optic neuropathy (LHON); Leigh disease (subacute necrotizing encephalomyelopathy); long-chain acyl-CoA dehydrogenase deficiency (LCAD); Luft disease; medium-chain acyl-CoA dehydrogenase deficiency (MCAD); mitochondrial cytopathy; mitochondrial encephalomyopathy tactic acidosis and stroke-like episodes (MELAS); mitochondrial encephalopathy; mitochondrial myopathy; multiple acyl-CoA dehydrogenase deficiency (MAD); glutaric aciduria Type II; myoclonic epilepsy and ragged-red fiber disease (MERRF); myoneurogastointestinal disorder and encephalopathy (MNGIE); neuropathy ataxia and retinitis pigmentosa (NARP); pearson syndrome; pyruvate carboxylase deficiency; pyruvate dehydrogenase deficiency (PHD); and short-chain acyl-CoA dehydrogenase deficiency (SCAD).

Other examples of mitochondrial conditions include respiratory chain disorders such as: Complex I: NADH dehydrogenase (NADH-COQ reductase) deficiency; Complex II: Succinate dehydrogenase deficiency; Complex III: ubiquinone-cytochrome c oxidoreductase deficiency; Complex IV: cytochrome c oxidase (COX) deficiency; and Complex V: ATP synthase deficiency.

In one embodiment, an organism such as a plant or animal can be diagnosed for the presence of a mitochondrial condition by genetically screening the organism. In some embodiments, the organisms' genetic DNA or mitochondrial DNA can be analyzed. In some embodiments, the organism's RNA, mRNA, siRNA, or cRNA is analyzed.

An organism having by or susceptible of having a mitochondrial condition can then be administered one or more of the compositions disclosed herein to modulate, treat, or prevent the condition. Such compositions include, but are not limited to: polypeptides comprising, consisting essentially of, or consisting of, any one or more of amino acid sequences SEQ ID NO: 1-349, or any one or more of amino acids sequences SEQ ID NOS: 50-244, 248-249, 257-349, or any one or more of amino acid sequences SEQ ID NO: 1-14, 50-244, 257-349, or any one or more of amino acid sequences SEQ ID NO: 1-2 or SEQ ID NO: 1 or an analog or homolog of any of the above; (ii) nucleic acids encoding any of the above polypeptides; (iii) an antibody or antibody fragment that specifically binds a polypeptide comprising any one or more amino acid sequences: SEQ ID NO: 1-304 or any one of amino acid sequences SEQ ID NOS: 1-14, 50-244, 257-349; and (iv) small molecules or analogs of the above polypeptides.

3. Inflammation

Acute and chronic pain is frequently associated with inflammation as a result of tissue destruction, abnormal immune reactivity or nerve injury.

In one aspect, the compositions herein can also be used to treat, modulate, or prevent inflammation in an organism. The inflammation can be due to a variety of external or internal insults, such as infectious agents, physical injury, hypoxia, or disease processes in nearly any organ or tissue in the body with one or more of the following symptoms: redness, heat, tenderness/pain, and swelling. Other examples are inflammatory diseases which the compositions herein can be used to treat include those such as rheumatoid arthritis, inflammatory bowel disease, scleroderma, cutaneous lupus erythematosus, systemic lupus erythematosus, type 1 and II diabetes, asthma, multiple sclerosis, abscess, wounds, meningitis, encephalitis, vasculitis, and cardiovascular diseases.

Since the discovery of salicylic acid (SA) as an anti-inflammatory compound and the subsequent synthesis of aspirin (ASA) over a century ago, several classes of structurally diverse compounds have become available for the treatment of human inflammatory disorders. These compounds are collectively known as NSAIDs and share with ASA a common mechanism by which they exert their anti-inflammatory action. Inflammation is now recognized as a type of immune response that directs immune system components to the site of injury or infection and is a major contributor to many diseases. Inflammation can be localized to a wound or an injury site and it can be systemic. Recent studies show a possible link between cardiovascular diseases and inflammation, e.g., the levels of C-reactive polypeptide, a molecular marker of inflammation, rank with cholesterol levels as indicators of future coronary heart disease.

In one aspect, the present invention relates to the use of the compositions herein including for example (i) polypeptides comprising, consisting essentially of, or consisting of, an amino acid sequence such as any one or more of SEQ ID NO: 1-349, or any one or more of SEQ ID NO: 1-14, 50-244, 248-249, 257-349, or SEQ ID NO: 1; (ii) nucleic acids encoding any of the above polypeptides; (iii) antibodies that specifically bind any of the above polypeptide; and (iv) small molecules or analogs of the above polypeptides.

In some embodiments, the compositions above can be used in combination with one or more other anti-inflammatory agents to relief the inflammation.

4. Neoplastic Growth

Cell division and growth are essential for development and repair of organs and tissues. However excess or uncontrolled growth is important causes of disease such as cancer. Endogenous opioid peptides have played a role in regulating immunity and tumor growth. In addition to their use in the treatment of pain, opioids, appears to be important in the growth regulation of normal and neoplastic tissue (Rasmussen et al., 2002, NEL. 23:193-198). For example, release of endogenous opioids has been found to stimulate growth of 1 breast cancer in rats and opiate receptor antagonists have reduced the growth of these tumors (Balslev et al., 1989, Am. J. Path., 134:473-479). In another example, cyclooxygenase-2 (COX-2) and the prostaglandins resulting from its enzymatic activity have also been shown to play a role in modulating cell growth and development of human neoplasia. Evidence includes a direct relationship between COX-2 expression and cancer incidence in humans and animal models, increased tumorigenesis after genetic manipulation of COX-2, and significant anti-tumor properties of NSAIDs in animal models and in some human cancers. Moreover, recent data showed that COX-2 and the derived prostaglandins are involved in control of cellular growth, apoptosis, and signal through a group of nuclear receptors named peroxisome proliferator-activated receptors (PPARs; Trifan and Hla, 2003, J. Cell. Mol. Med. 7:207-222; Martinsgreen et al., 1994, Cancer Res. 54:4334-4341).

Thus, any of the compositions herein can also be used for the treat, prevent or modulate aberrant cell growth and in particular, cancer.

Non-limiting examples of cancers that may be modulated, treated, or prevented by the compositions herein include, but are not limited to, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, reticulum cell sarcoma, myeloma, giant cell tumor, small-cell lung tumor, gallstones, islet cell tumor, primary brain tumor, acute and chronic lymphocytic and granulocytic tumors, hairy-cell leukemia, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforme, leukemias, lymphomas, malignant melanomas, epidermoid carcinomas, and other carcinomas and sarcomas.

The largest class of tumors falls into the ectoderm/endoderm class. This class includes the leading causes of death in humans (bronchogenic carcinoma, colon adenocarcinoma, breast carcinoma and prostate carcinoma and the most frequently occurring (though usually non-lethal) tumors of humans (squamous cell carcinoma of skin and basal cell carcinoma of skin). The other tumor groups are tumors of mesodermal lineage (including all sarcomas) and tumors of neuroectodermal lineage.

Thus, in some embodiments, a composition herein (e.g., SEQ ID NO: 1) can be administered to a subject susceptible of or having cancer to treat, modulate, or prevent the condition. Such compositions include for example (i) polypeptides comprising, consisting essentially of, or consisting of, an amino acid sequence such as any one or more of SEQ ID NOS: 1-349 or any one or more of SEQ ID NOS: 1-244, 248-249, 257-349, or any one or more of SEQ ID NO: 1-14, 50-244, 257-349, or any one or more of SEQ ID NO: 1, 153, 304-349 or any one or more of SEQ ID NO: 1; (ii) nucleic acids encoding any of the above polypeptides; (iii) an antibody or antibody fragment that specifically binds any of the above polypeptide, for the treatment of inflammatory conditions; and (iv) small molecules or analogs of the above polypeptides.

Such compositions can be administered along with one or more anti-neoplastic agents or be co-formulated with one or more anti-neoplastic agents to increase their therapeutic effect.

Anti-neoplastic agents can be grouped into the following general categories: alkylating agents, anti-metabolites, mitotic inhibitors, anti-neoplastic antibiotics, hormonal agents, and miscellaneous. Example for an alkylating agent is Mechlorethamine hydrochloride that is used to treat Hodgkin's disease in man. Example for antimetabolites is methotrexate, an inhibitor of dihydrofolate reductase. Examples for mitotic inhibitors are Paclitaxel and docetaxel that are antimicrotubule agents. Examples for antineoplastic antibiotics are Mitoxantrone, an anthracenedione related to the anthracycline antibiotics, Doxorubicin and Bleomycin. Examples for hormonal agents are glucocorticoids.

Additional examples of antineoplastic agents include, but are not limited to: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Imofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycinl, Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

5. Innate Immune System

An organism has inborn defense mechanisms or innate immune system that allows it to defend itself against invasions by pathogens. The compositions herein can also be used to modulate, prevent and/or treat pathogen invasions (bacteria, virus, and fungi, crop pests, etc.) in humans and plants.

In humans, the few microbes that manage to cross the barriers of skin, mucus, cilia, and pH are usually eliminated by innate immune system, which commence immediately upon pathogen entry. If phagocytosis cannot rapidly eliminate pathogen, inflammation is induced with the synthesis of cytokines and acute phase polypeptides. This early-induced response is not antigen-specific. Only if the inflammatory process is unsuccessful at eliminating pathogen, the adaptive immune system is activated.

Plants also possess the mechanism of self-defense against pathogens and other abiotic stresses. (Cohen et al., (2001) Curr. Opin. Immunol. 13:55-62) Salicylic acid plays an important role in the induction of resistance to a broad spectrum of widely different pathogens such as fungi, bacteria or viruses such as the bacteria Pseudomonas syringae and the tobacco mosaic virus (TMV). While conventional pesticides targets the pathogens, most non-conventional pest control chemicals (biopesticides) are based on small molecule production either by added genetic material or microorganisms, which increases a plants ability to fight pathogens.

Thus, in some embodiments, the compositions herein are used to increase a seed, plant (e.g., crop) or plant cuttings' innate immune response to pathogen (e.g., bacteria, viruses, fungi, crop pests). This can help reduce crop losses. Other examples of conditions that may result in crop losses that can be preventable or diminished by the compositions herein include stress conditions such as drought, freezing or reduced temperatures, and other unfavorable environmental conditions (see discussion of temperature regulation above).

Examples of plants that may be treated with the compositions herein include, culture plans such as wheat, barley, rye, oats, rice, sorghum and the like; including Chenopodiaceae, e.g., sugar beet and fodder beet; pome and stone fruits and berries, e.g., apples, pears, plums, peaches, almonds, cherries, strawberries, raspberries and blackberries; Legume, e.g., beans, lentils, peas, soy beans; Brassicaceae, e.g., rape, mustard, cabbages and turnips. Cucurbitaceae, e.g., pumpkins, gherkins, melons, cucumbers, squashes; fibrous plants, e.g., cotton, flax, hemp, jute; citrus fruits, e.g., orange, lemon, grapefruit, mandarin; vegetables, e.g., spinach, lettuce, asparagus, ground-nuts; carrots, onions, tomatoes, potatoes, hot and sweet peppers; laurel-like plants, e.g., avocado, cinnamon, camphor tree; or plants such as maize, tobacco, nuts, coffee, sugar-cane, tea, vines, hops, bananas, rubber plants, poppy, olive, sunflower, coconut, castor-oil plant, cocoa as well as ornamental plants, e.g., flowers, shrubs, deciduous trees and evergreen trees such as conifers. This list is given with the purpose of illustrating the invention and not to delimiting it thereto.

Thus, in some embodiments, a composition herein can be administered to a plant or animal to prevent or treat a pathogen invasion. Such compositions include for example (i) polypeptides comprising, consisting essentially of, or consisting of, an amino acid sequence such as any one or more of SEQ ID NOS: 1-349 or any one or more of SEQ ID NOS: 1-244, 248-249, 257-349, or any one or more of SEQ ID NO: 1-14, 50-244, 257-349, or any one or more of SEQ ID NO: 1, 153, 304-349 or any one or more of SEQ ID NO; 1; (ii) nucleic acids encoding any of the above polypeptides; (iii) an antibody or antibody fragment that specifically binds any of the above polypeptide, for the treatment of inflammatory conditions; and (iv) small molecules or analogs of the above polypeptides. Such compositions can further include a veterinary excipient, pharmaceutical excipient or agricultural excipient.

6. Neurological Condition

The present invention contemplates treating or preventing a neurological and/or nuerodegenerative condition using one or more of the compositions herein. Such compositions include for example (i) polypeptides comprising, consisting essentially of, or consisting of, an amino acid sequence such as any one or more of SEQ ID NOS: 1-349 or any one or more of SEQ ID NOS: 1-244, 248-249, 257-349, or any one or more of SEQ ID NO: 1-14, 50-244, 257-349, or any one or more of SEQ ID NO: 1, 153, 304-349 or any one or more of SEQ ID NO: 1; (ii) nucleic acids encoding any of the above polypeptides; (iii) an antibody or antibody fragment that specifically binds any of the above polypeptide, for the treatment of inflammatory conditions; and (iv) small molecules or analogs of the above polypeptides.

Examples of neurological and neurodegenerative conditions that may be modulated, treated, or prevented by the compositions herein include, but are not limited to, anxiety disorder, panic disorder, obsessive-compulsive disorder, post-traumatic stress disorder, social phobia (or social anxiety disorder), specific phobias, and generalized anxiety disorder. Any of the above conditions can also be accompanied by or manifested by other conditions such as depression, drug abuse, alcoholism, Aicardi syndrome, Alzheimer's disease, amnesia, amyotrophic lateral sclerosis (Lou Gehrig's disease), anencephaly, aphasia, arachnoiditis, Arnold Chiari malformation, Batten disease, Bell's Palsy, brachial plexus injury, brain injury, brain tumors, Charcot-Marie tooth disease, dystonia, encephalitis, epilepsy, essential tremor, Guillian-Barre syndrome, hydrocephalus, hyperhidrosis, Krabbes disease, leukodystrophy, meningitis, Moebius syndrome, multiple sclerosis, muscular dystrophy, Parkinson's disease, peripheral neuropathy, postural orthostatic tachycardia syndrome, progressive supranuclear palsy, prosopagnosia, shingles, Shy-Drager syndrome, spasmodic torticollis, spina bifida, spinal muscular atrophy, stiff man syndrome, synesthesia, syringomyelia, thoracic outlet syndrome, tourette syndrome, toxoplasmosis, and trigeminal neurolagia.

For example, a composition comprising a polypeptide comprising any one or more of SEQ ID NOs: 1-349 or a small molecule thereof, or a nucleic acid encoding the above can be used to treat a neurological and/or nuerodegenerative condition such as Alzheimer's disease. Polymorphs, salts, metabolites, and prodrugs of the above are also contemplated herein.

7. Addiction

Any of the compositions herein can also be used to treat an addiction in an animal. Such compositions include for example (i) polypeptides comprising, consisting essentially of, or consisting of, an amino acid sequence such as any one or more of SEQ ID NOS: 1-349 or any one or more of SEQ ID NOS: 1-244, 248-249, 257-349, or any one or more of SEQ ID NO: 1-14, 50-244, 257-349, or any one or more of SEQ ID NO: 1, 153, 304-349 or any one or more of SEQ ID NO: 1; (ii) nucleic acids encoding any of the above polypeptides; (iii) an antibody or antibody fragment that specifically binds any of the above polypeptide, for the treatment of inflammatory conditions; and (iv) small molecules or analogs of the above polypeptides.

In some cases, a composition comprising SEQ ID NO: 1, or a small molecule thereof is used to treat an addiction.

In some cases, the composition further comprises one or more antagonists.

Examples of antagonists include those selected from the group consisting of Alvimopan, trans-3,4-dimethyl-4-(3-hydroxyphenyl)piperidine; ANTI, 5′-acetamidinoethylnaltrindole; 4-Aminoquinoline; N-(4-amino-2-methylquinolin-6-yl)-2-(4-ethylphenoxymethyl)benzamide monohydrochloride; Benzimidazolinone; 7-Benzylidenenaltrexone; Binaltorphimine; nor-Binaltorphimine; Butorphanol (17-cyclobutylmethyl-3,14-dihydroxymorphinan) tartrate; CTAP, D-Phe-Cys-Tyr-D-Trp-Arg-Thr-Pen-Thr-NH2; CTOP, D-Phe-Cys-Tyr-D-Trp-Orn-Thr-Pen-Thr-NH2; Cyclazocine; Cyprodime; 1,3-Dimethyl-4-piperidinone; Ethylketocyclazocine; β-Funaltrexamine; GNTI, 5′-Guanidinonaltrindole; ICI 174864, N,N-diallyl-Tyr-Aib-Aib-Phe-Leu; Indolomorphinan; 5¢-Isothiocyanate; J-113397, 1-[(3R,4R)-1-Cyclooctylmethyl-3-hydroxymethyl-4-piperidyl]-3-ethyl-1,3-dihydro-2H-benzimidazol-2-one; JDTic, (3R)-7-Hydroxy-N-[(1S)-1-[[(3R,4R)-4-(3-hydroxyphenyl)-3,4-dimethyl-1-piperidinyl]methyl]-2-methylpropyl]-1,2,3,4-tetrahydro-3-isoquinoline-carboxamide 3-Quadazocine; Loperamide; Methoxynaltrexone; Methylnaltrexone; Mr 2266; Nalmefene; Nalorphine; Naloxone; Naloxone methiodide; Naloxazone; Naltrexone; β-Naltrexamine; Naltriben; Naltrindole; Phenylpiperidine; SB-612111, (−)-cis-1-methyl-7-[[4-(2,6-dichlorophenyl)piperidin-1-yl]methyl]-6,7,8,9-tetrahydro-5H-benzocyclohepten-5-ol; SoRI 9409, 59-(4-chlorophenyl)-17-(cyclopropylmethyl)-6,7-didehydro-3,14-dihydroxy-4,5a-epoxypyrido-[29, 39:6,7]morphinan, SNC 80; TIPP-y, Tyr-Tic-Phe-Phe; Triethyleneglycolnaltrexamine.

The antagonist can be co-formulated with the compositions herein or co-administered (administered separately).

Non-limiting examples of addictions that can be treated (prevented, reduced, or cured) using the methods herein include alcoholism, addiction to cocaine, addiction to morphine, addiction to heroine, and addiction is to a painkiller.

### Screening Assays

The present invention also contemplates screening assays designed to identify agents that alter (e.g., increase or decrease in a statistically significant manner) one or more of the compositions herein (e.g., a polypeptide comprising, consisting essentially of, or consisting of an amino acid sequence of (or encoded by) SEQ ID NOs: 1-349, or any of the above with a methionine at the N-terminus). The present invention also contemplates screening assays that identify small molecules or analogs of the compositions herein (e.g., any one of SEQ ID NOS: 1-349). Finally, the present invention contemplates assays that identify targets of the compositions herein (e.g., SEQ ID NOS: 1-349).

For example, in certain embodiments, when the compositions herein are contacted with a known polypeptide in the absence and presence of a candidate agent and under conditions and for a time sufficient for binding to the polypeptide to occur, and the effect of the agent on the binding interaction between the polypeptide and a composition herein is determined. A candidate agent may alter any of the herein described parameters directly (e.g., by physical contact with the polypeptide at a site of ligand binding) or indirectly (e.g., by interaction with one or more proximal or distal sites within the polypeptide, as may according to non-limiting theory alter the described parameter by interacting with other than a site of ligand binding, for instance, electron transfer or UV absorbance, or changing the conformation of the polypeptide. In some embodiments, the candidate agent may be a peptide, polypeptide, polypeptide or small molecules, and in certain preferred embodiments the candidate agent may be a structural mimetic of one or more of the compositions herein. Typically, and in more preferred embodiments such as for high throughput screening, candidate agents are provided as “libraries” or collections of compounds, compositions or molecules. Such molecules typically include compounds known in the art as “small molecules” and having molecular weights less than 10⁴ daltons, preferably less than 10⁵ daltons. For example, members of a library of test compounds can be administered to a plurality of samples, each containing at least one homolog of a polypeptide herein and a known polypeptide as provided herein, and then assayed for their ability to alter at least one of the above-described parameters.

In another example, small molecules are screened to identify ones that interact with or mimic (have similar 3D structure) any one or more of SEQ ID NOS: 1-349. Candidate agents can be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides. Those having ordinary skill in the art will appreciate that a diverse assortment of such libraries may be prepared according to established procedures, and tested using the known polypeptides as a target.

There are a variety of assay formats known to those of ordinary skill in the art for detecting binding interactions between polypeptides and their cognate ligands. See, e.g., Harlow and Lane, 1988 In: Antibodies: A Laboratory Manual, Cold Spring Harbor Laboratory. Within one embodiment, a polypeptide or polypeptide is immobilized on a solid support prior to contact with the ligand. Binding may then be detected using a detection reagent that specifically binds to the polypeptide, for example, at a site known or suspected of being a site of ligand interaction (e.g., an antibody or fragment thereof, or using a detectable portion of the polypeptide (e.g., direct detection of a UV-absorbing moiety, or detection of electron transfer to an acceptor molecule).

A solid support may be any material known to those of ordinary skill in the art. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. A polypeptide may be immobilized on the solid support using a variety of techniques known to those of skill in the art, which are amply described in the patent and scientific literature. In the context of the present invention, the term “immobilization” refers to both non-covalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time.

Binding is generally allowed to occur under solution conditions and for an amount of time sufficient to detect the bound ligand. An appropriate amount of time may generally be determined by assaying the level of binding that occurs over a period of time. After incubating under conditions and for a time sufficient to permit interaction of a polypeptide of the invention and candidate receptor agent, the level of the ligand-receptor binding is detected and compared to the level of binding in the presence and absence the polypeptide of the invention.

For example, following a suitable interval for competitive ligand binding, unbound ligand is removed, and bound ligand is detected using a linked reporter group or a separate detectable marker comprising a reporter group. The method employed for detecting binding depends upon the nature of the reporter group employed. When electron transfer is detected, fluorescence or colorimetric or other techniques may be used. For radiometric quantification of ligand binding (or, e.g., competitive inhibition by a candidate agent of the binding site for a polypeptide comprising, consisting essentially of, or consisting of an amino acid sequence of (or encoded by) any one or more SEQ ID NOs: 1-349, or any of the above with a methionine at the N-terminus, of a detectably labeled ligand comprising a radioactive group), scintillation counting or auto-radiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

An agent that binds to a polypeptide of the invention and/or to a polypeptide complex comprising a polypeptide of the invention may result in a detectable decrease or increase in binding the polypeptide to its natural receptor. Such altered levels of ligand-receptor binding can be detected by a statistically significant increase or decrease in binding to the receptor. Such agents that interfere with the ligand-receptor binding may be used as inhibitors of the compositions herein.

One or more of above peptides can be used to screen small molecules and other compounds (e.g., antibodies, peptides, peptide nucleic acids, and nucleic acids) that interact with any one or more of SEQ ID NOs: 1-349. Such library of compounds can include at least 1, 2, 3, 4, 5, 6, 7, 8, 9, 10, 15, 20, 30, 40, 50, 60 or 70 agents.

The present invention provides compositions, methods and kits for use in a phage display peptide library in which a library of analogs or variants of one or more of SEQ ID NOs: 1-349, is expressed on the outside of a phage virion, and the DNA encoding each variant or analog resides inside the virus. This creates a physical linkage between each variant protein sequence and the DNA encoding it, which allows rapid partitioning based on binding affinity to a given target molecule (antibodies, enzymes, cell-surface receptors, etc.) by an in vitro selection process called panning (Whaley et al., 2000, Nature 405:665-668).

The present invention also provides methods for identifying lead compounds for treatment of mitochondrial and metabolic conditions such as pain, inflammation, fever, Alzheimer's disease and any other disease mentioned herein. Such methods involve the use of a thermogenic plant for studying thermoregulation activity by candidate agents. Examples of thermogenic plants include those such as the Sauromatum guttatum, members of the Araceae family, Amorphophallus konjac, Arum italicum, A. dioscoridis, Dracunculus vulgaris; lotus (Nelumbonaceae), Dutchman's pipes (Aristolochiaceae), palms (Arecaceae and Cyclanthaceae), custard apples (Annonaceae), magnolias (Magnoliaceae), Illicium (Illiciaceae), Rafflesia (Rafflesiaceae), winter's bark (Winteraceae) and cycads (Cycadaceae).

In one example, using Sauromatum guttatum as the experimental module, on the day of inflorescence-opening, the Sauromatum appendix (a 20-40 cm-long, slender organ) becomes warm reaching a 32° C. temperature (Skubatz et al., 1991, Plant Physiol. 95:1084-1088). The heat generated by the appendix is generated by the mitochondria. This mitochondrial activity can be triggered by the addition of phenolic compounds, including but not limited to, salicylic acid, aspirin, and 2,6-dihydroxybenzoic acid. Test agents can be applied to the plant and the plant's temperature may be monitored in vivo.

Test agents (e.g., polypeptide, such as those disclosed herein) that can module (increase, decrease, sustain, or shorten) heat generated by the plants can be used as lead compounds in animal models to test their ability to treat/modulate a metabolic condition. In some embodiments, small molecules or mimetics of the test compounds are applied to the plants. A small molecule or mimetic that has the same effect as a test agent such as a polypeptide may be then tested in an animal model for its ability to treat/modulate a metabolic condition. In some embodiments, such test compounds are agonists, antagonists and/or other modulators of mitochondrial activities.

### Administration

The compositions (including formulations) herein can be administered systemically or locally to a plant or animal by any means known in the art. For example, to an animal such as a human, the compositions herein can be administered parenterally (which includes subcutaneously, intravenously, intramuscularly, intrasternally, intracavernously, intrathecally, and intraurethrally), intracranially, intraorbitally, intracapsularly, intraspinally, intracistemally, intrapulmonaryly (via inhalation), orally, intravenously, intra-arterially, intramedullary, intrathecally, intraventricularly, intrameatally, transdermally, subcutaneously, intraperitoneally, intranasally, enterally, vaginally, sublingually, or rectally. Preferably, the compositions herein are administered to an animal topically, subdermally or intravenously. In some embodiments, the composition/formulations herein are administered using insert(s), bead(s), timed-release formulation(s), patch(es) or fast-release formulation(s).

For plants, the compositions herein can be administered by any method known in the art, including, but not limited to, spray, drip irrigation or other irrigation, dipping at least a portion of said plant or seed in said composition, coating at least partially said plant or seed with said composition, etc. In another embodiment, a nucleic acid sequence encoding any of the above compositions can be used to transfect plants such that their heat production is regulated.

The compositions/formulations herein are preferably administered in an effective dose. It will be evident to those skilled in the art that the number, frequency, and duration of administration will be dependent upon the response of the host.

For therapeutic delivery, agents at concentrations of about 0.01 μg/kg to about 1000-mg/kg-body weight may be administered, typically by the intradermal, subcutaneous, intramuscular or intravenous route, or by other routes. A preferred dosage is about 1 μg/kg to about 1000 mg/kg, or about 5 μg/kg to about 500 mg/kg, or about 10 μg/kg to about 100 mg/kg.

For agricultural delivery, agents may be administered at a concentration that is agriculturally therapeutically effective, e.g., about 50-3000 grams per hectare, preferably from about 50-1500 gram per hectare, and more preferably from about 150-300 gram per hectare. Assuming a composition is comprised of 100% active ingredients, then, in general, the amount of the subject composition used will range from about 0.005% to 25% of the weight of the seed, and more preferably, from about 0.01% to about 10% of the weight of the seed. In yet another embodiment the amount of the subject composition used will be in the range of 0.01% to 1% of the active ingredients relative to the weight of the seed, or 0.05% to 0.5%.

### EXAMPLES

### Example 1

A total of forty (40) male Sprague Dawley rats (Harlan Sprague Dawley Inc., Indianapolis, Ind., USA) were used in the study. The rats are specific pathogen free and approximately 250 grams upon arrival. The rats were housed in the vivarium in clear polycarbonate plastic cages (48×27×20 cm); 2 rats per cage until a few days prior to surgery at which point they were singularly housed for the remainder of the study procedures. The bedding material is irradiated corn-cob bedding (Bed-O-Cob, The Andersons, Maumee, Ohio, USA) that was changed weekly. The rats were acclimated for two weeks prior to the commencement of the experimental procedures. The room in which the rats were housed throughout the study was supplied with HEPA filtered air at the rate of 10-15 air changes per hour. The temperature was maintained at 18-26° C. with a relative humidity of 30-70%. Illumination is approximately 300 lumens/m² at 1 m above floor level on a 12-hour light/dark cycle. The rats had ad libitum access to oval pellet Certified Picolab Rodent Diet 20 (PMI Feeds Inc., Richmond, Ind., USA) and deionized water.

All rats were anesthetized with Inhalation anesthetic (Isoflurane). The plantar aspect of the foot was cleaned and prepped for aseptic surgery. The animals were placed in ventral recumbancy. A 1-246 cm longitudinal incision was made with a #11 blade, through the skin and fascia of the plantar aspect of the foot, starting 0.5 cm from the proximal edge of the heel and extending towards the toes. The plantaris muscle was elevated and incised longitudinally. The muscle origin and insertion remained intact. Gentle pressure was applied for hemostatis, if needed. The skin was closed with suture material and the wound site covered with a triple antibiotic ointment. The rats were allowed to recover in their cage until regaining full mobility.

On days 1 and 3 post-surgery, animals were dosed with the appropriate test or control compound (Table III).

TABLE III
Treatment groups for the first study of the analgesic property
of SEQ ID NO: 1 on post-operative pain in rats.

| Group No. | Surgery | Treatment | Dose | | Route |
|---|---|---|---|---|---|
| 1 | Sham (No surgery) | Vehicle | 0.1 | mL | Topical |
| | | 0.17% DMSO, 0.05% Silwet | | | |
| | | in H2O | | | |
| 2 | Brennan model | Vehicle | 0.1 | mL | Topical |
| 3 | Brennan model | Morphine in PBS | 5 | mg/kg | Subcutaneous |
| 4 | Brennan model | SEQ ID NO: 1 in vehicle | 150 | μg/0.15 mL | Topical |
| 5 | Brennan model | SEQ ID NO: 1 in PBS | 150 | μg/0.1 mL | Subdermal |

On days 1 and 3 post-surgery, the rats underwent Von Frey testing for mechanical allodynia. Tactile sensitivity (i.e. mechanical allodynia) was measured using calibrated filaments touched to the plantar surface of the affected limb.

Procedurally, the rats were placed in a plastic cage with a wire mesh bottom and allowed to acclimate for 5 to 10 minutes. Once the animals settled, the plantar surface of the right hind paw was touched with a 2.0 g von Frey filaments. In the absence of a paw withdrawal response to the initially selected filament, a stronger stimulus was presented; in the event of paw withdrawal, the next weaker stimulus was chosen. In this fashion, the resulting pattern of positive and negative responses was used to determine the paw withdrawal threshold.

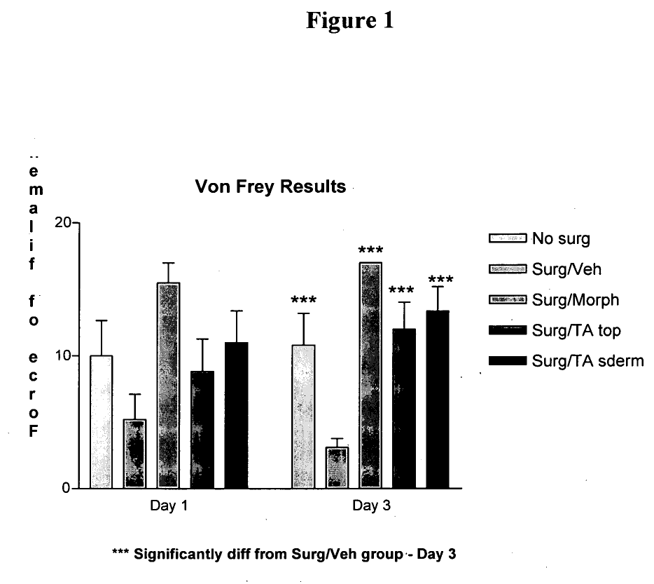

FIG. 1. Data were analyzed using a one-way ANOVA followed by Newman-Keuls' Multiple Comparison. Statistical significance was p<0.05. It shows that SEQ ID NO: 1 was able to significantly reduce pain. Abbreviations: TA, test article, which is SEQ ID NO: 1; sderm, sub-dermal injection; top, topical application; veh, vehicle; surg, surgery; morph, morphine. The rats appeared relaxed and with less anxiety.

### Example 2

In a second independent study the rat hind paw withdrawal sensitivity was again evaluated after 3 h and 3 day post-surgery when the rats had received SEQ ID NO: 1 at three different concentrations: 1, 15 and 50 mg/kg. The dosing route was subdermal injection adjacent to the wound site. The rats received one dose 3 hours after the surgery and on Day 3 post-surgery, group 3 was dosed again as described in the Table IV. Pain measurements were taken 15-20 min after application of the drug. Baseline pain behavior was measured as follows: Withdrawal responses to mechanical stimulation were determined using von Frey filaments applied from underneath the cage through openings (12×12 mm in the plastic mesh floor to an area adjacent to the intended incision. Each von Frey filament (Target force of 0.008 g to 300 g) was applied once starting with 0.008 g filament and continuing until a withdrawal response occurred or 300 g force was reached. The median force producing a response, determined from three tests given over a 10-min period was considered the withdrawal threshold.

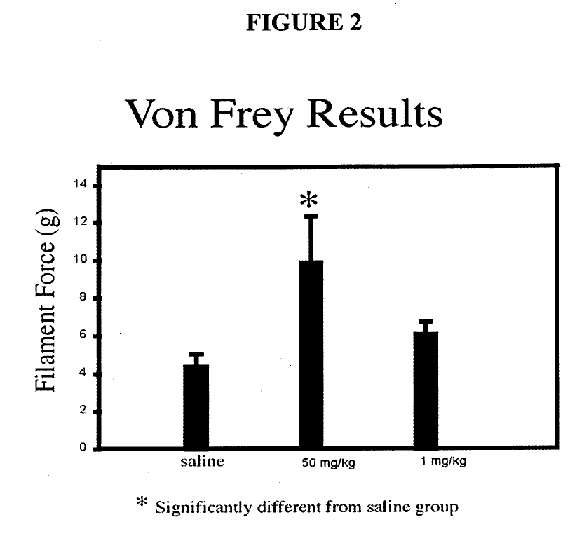

FIG. 2 illustrates withdrawal results show that SEQ ID NO: 1 has analgesic and property 3 h after surgery. In the saline treated animals, the control, 3-hour post-surgery were 25.1, 4.4 and 12.7 g, indicating significant hyperalgesic response immediately after surgery which subsided by Day-3. In the animals treated with 50 and 15 mg/kg doses of SEQ ID NO: 1 subdermally, the pre-dose, pre-surgery responses were 30.2 and 28.1 g, which were comparable to the saline treated group indicating uniformity of the pain response in all the three groups. The withdrawal response in the 50 mg/kg SEQ ID NO: 1 administered on Day-1 was 9.9 g compared to the 4.4 g for the saline group at the same time-point, indicating a 125% effect. The data expressed as mean±SE, were analyzed using ANOVA followed by Tukey-HSD Multiple Comparison Test. Statistical significance was p<0.05.

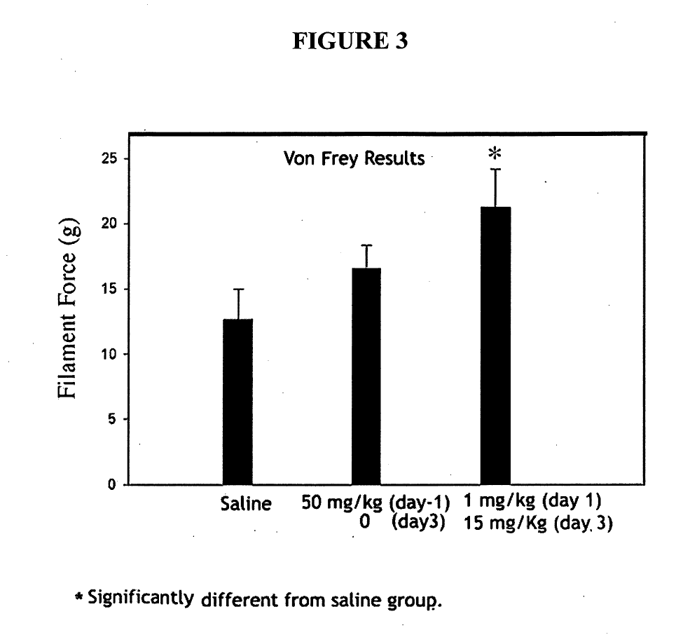

FIG. 3. Withdrawal results show that SEQ ID NO: 1 has analgesic and property on Day 3 post-surgery. The group, which did not receive any further dosing showed a 31% increase in response time on Day 3, compared to the saline group at the respective time point. In the group of animals administered with 1 mg/kg NPL/PA2 on day 1 post-surgery, the 3-hour post surgery measurements showed a 39% increase in the pain threshold. On Day-3, when these animals were administered with 15.25 mg/kg of SEQ ID NO: 1 subdermally, the withdrawal response was at 21.3 g, compared to the withdrawal response of saline group at 12.7 g, constituting an increase of 68%. The data expressed as mean±SE, were analyzed using ANOVA followed by Tukey-HSD Multiple Comparison Test. Statistical significance was p<0.05.

TABLE IV
Treatment groups for the first study of the analgesic property
of SEQ ID NO: 1 on post-operative pain in rats.

| | Number of | | | | |
|---|---|---|---|---|---|
| | Animals | | | Concentration | |
| Group | Male | Route | Time of Dosing | (mg/kg) | |
| 1 | 10 | saline, vehicle | Day 1, 3 h after | 0 | |
| | | | surgery | | |
| 2 | 10 | subdermal | Day 1, 3 h after | 50 | mg/kg |
| | | | surgery | | |
| 3 | 10 | subdermal | Day 1, 3 h after | 1 | mg/kg |
| | | | surgery | | |
| | | | Day 3 | 15 | mg/kg |

### Example 3

An adult male subject suffering from chronic pain as a result from of multiple fractures to both tibia and fibula in both legs and severe traumatic soft tissue damage was topically administered a composition containing a polypeptide of SEQ ID NO: 1 at regions experiencing pain (mostly knees and ankles). The pharmaceutical formulation administered comprised of 5 μM of SEQ ID NO: 1 in 0.01% Silwet L-77. Relief was noticed within 15 min. after administration of a single dose of about 5 μg/10 cm2 of SEQ ID NO: 1. The relief lasted for more than a week. A scaled score of 1 to 10 was used to evaluate treatment efficacy where a score of 10 represented a patient with sever pain discomfort and complete inability to walk. A scaled score of 1 represented a patient experiencing no pain and able to freely walk or move the legs. Prior to treatment, the patient scored an 8-9 representing chronic pain and difficulties in walking long distance. After the treatment, the patient scored a 2-3 representing a significant decrease in pain. Treatment efficacy lasted for about 10 days.

### Example 4

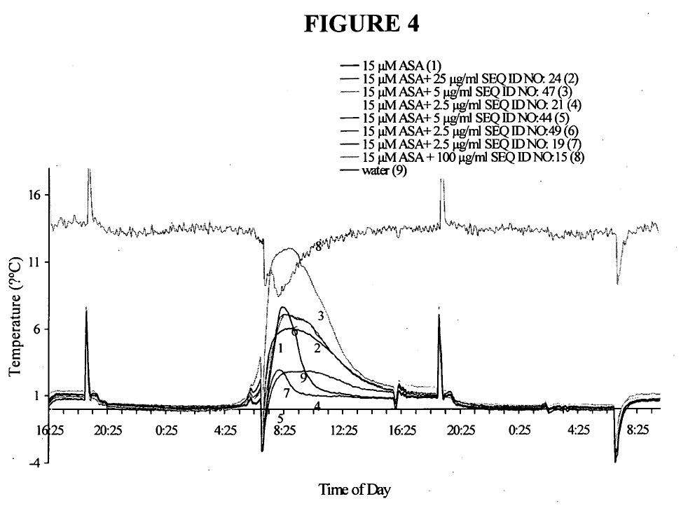

FIG. 4 illustrates modulation of heat generated by aspirin (ASA) in Sauromatum guttatum appendix in the presence of various opioid peptides and the Alzheimer's peptide, Aβ 1-42. One day before heat-production, sections of the appendix were placed in different aqueous solutions containing ASA with or without an opioid peptide. [Leu]-Enkephalin, SEQ ID NO: 15; Human β-Endorphin; SEQ ID NO: 19; Dynorphin A, SEQ ID NO: 21; Endomorphin 2, SEQ ID NO: 24; Neuropeptide AF, SEQ ID NO: 44; β-human-Casomorphin, SEQ ID NO: 47; Alzheimer's peptide, SEQ ID NO: 49. Sections of the appendix were placed in distilled water that was not generated any heat is the control (9). Temperature was recorded with thermocouples attached to the section every 5 min. The y-axis is the appendix temperature above ambient and the x-axis shows the time of the day. This figure illustrates that opioid peptides and the neurotoxic Alzheimer's peptide, Aβ 1-42 can modulate thermogenicity in plants as well as animals and act as mitochondrial modulators.

### Example 5

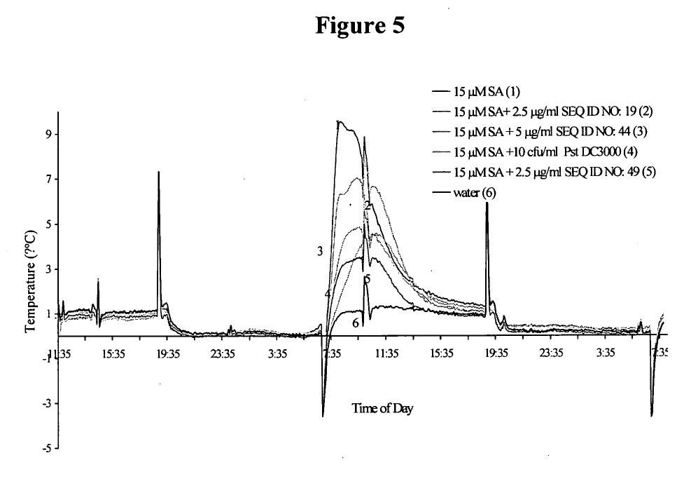

FIG. 5 illustrates modulation of heat generated by salicylic acid (SA) in the presence of human opioid peptides (β-Endorphin, SEQ ID NO: 19 and Neuropeptide AF, SEQ ID NO: 44) and, the Alzheimer's peptide, Aβ 1-42, SEQ ID NO: 49, and, a plant virulent bacterial pathogen (Pst DC3000). One day before heat-production, sections of the appendix were placed in different aqueous solutions containing salicylic acid (SA) with or without a peptide or with the bacterial plant pathogen, Pseudomonas syringae pv. Tomato, DC3000. Sections from the appendix were placed in distilled water that was not generated any heat is the control (water). Temperature was recorded with thermocouples attached to the sections every 5 min. The y-axis is the appendix temperature above ambient and the x-axis shows the time of the day. This figure illustrates that these peptides modulate thermogenicity in plants as well as animals and act as mitochondrial modulators.

### Example 6

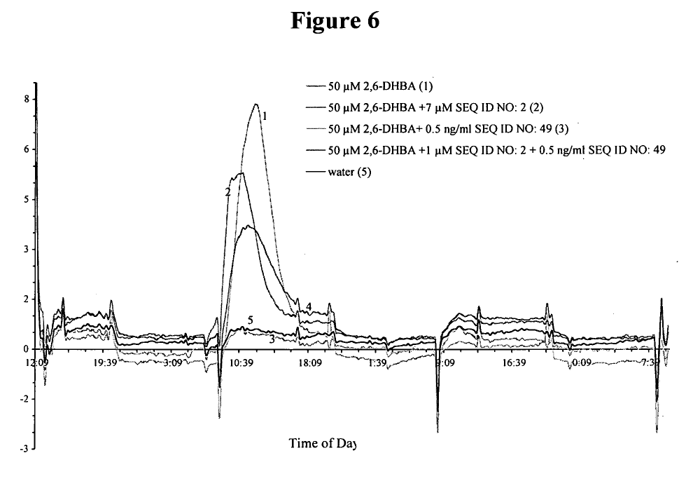

FIG. 6 illustrates modulation of heat generated by 2,6-dihydroxybenzoic acid in the presence of the Alzheimer's peptide, Aβ 1-42, SEQ ID NO: 49 and a plant gene derived sequence in the Sauromatum guttatum appendix. One day before heat-production, sections of the appendix were placed in different solutions containing 2,6-dihydroxybenzoic acid (2,6-DHBA) with or without SEQ ID NO: 2 (FLPSEFGVDVDR) and SEQ ID NO: 51. Heat-production was monitored with thermocouples attached to the sections. A section placed in distilled water that was not generated any heat is the control (water). The y-axis is the appendix temperature above ambient and x-axis shows the time of the day. The temperature was recorded every 5 min. Moreover, in a second experiment, application of SEQ ID NO: 2 up to 50 μM did not generate heat (data not shown). This figure illustrates that these peptides modulate thermogenicity in plants as well as animals and act as mitochondrial modulators.

### Example 7

Administration of 10 μm of SEQ ID NO: 1 in 0.01% Silwet L-77 to Arabidopsis thaliana plants induces early flowering and abundance of flowers and pods.

### Example 8

Seeds of Arabidopsis ecotype Columbia (Col-0) were planted in potting soil. Plants were cultivated in a growth chamber with 10-h d (200 μmol m⁻²s⁻¹ at 22° C.) and 14-h night (18° C.) cycles and 80% RH. Once a week plants were supplied with water and modified one-half strength Hoagland nutrient solution: 2 mM KNO 5 mM Ca(NO₃)₂ and trace elements, pH 7.

A virulent plant pathogen, Pst Pseudomonas syringae pv tomato, DC3000 was grown at 28° C. on King's medium B containing 40 mg/L tetracycline. Plants were inoculated with 1×10⁷ cfu/ml of the pathogen in 0.01% Silwet L77 (v/v) (a surfactant) and distilled water 5 weeks after sowing. The bacterial suspension or a control solution (0.01% Silwet L77 in water) was then sprayed on the plant once.

Disease symptoms in Arabidopsis are water-soaked, spreading lesions, sometimes surrounded by chlorotic margin that eventually lead to yield loss and plant death.

To test the efficacy of SEQ ID NO: 15, some healthy and some disease plants were sprayed with salicylic acid in 0.01%. Silwet L77; some healthy and some disease plants were sprayed with SEQ ID NO: 15 in 0.01%. Silwet L77, and some healthy and some disease plants were sprayed with salicylic acid in combination with SEQ ID NO: 15 in 0.01%. Silwet L77. A control group of some healthy and some disease plants were sprayed with water in 0.01%. Silwet L77.

Symptoms of plants were classified by the percentage of infected leaves and the severity of the infection.

It was noted that application of 20 μl salicylic acid at a concentration of 1 mM and 20 μl SEQ ID NO: 15 to the leaves is enough to trigger systemic resistance to the virulent bacteria concentration.

### Example 9

### Original of Peptides

The human preprodynorphin is encoded by the PYND gene known also as preproenkephalin B gene (Horikawa et al. 1983). The 28-kD precursor, preprodynorphin, is post-translationally cleaved to form 5 secreted opioid peptides: beta-neoendorphin, dynorphin, leu-enkephalin, rimorphin, and leumorphin. The precursor consists of 254 aa with a signal sequence (region 1-20 aa) that precedes a conserved region of about 50 residues; a variable-length region; and the sequence of the neuropeptides (Beta-neoendorphin, 175-183; Dynorphin 207-223; Leumorphin, 226-254; Rimorphin, 226-238; Leu-enkephalin 226-230). These peptides are ligands for the kappa-type of opioid receptor. SEQ ID NO: 1 identified herein is residues 91-94 in the variable region. This region does not contain any known active peptides.

Incision activates the nociceptive system as a result of receptor stimulation by tissue injury. A reduction in the threshold of the nociceptors that transfer input from peripheral targets (skin, muscle, joints and the viscera) to the spinal cord and CNS causes the pain perception in the brain (Treede 1995; Brennan 1999). It has been shown that the receptive fields of dorsal horn neurons develop exaggerated responses to mechanical stimuli after plantar incision (Brennan 1999; Zahn et al. 2002). This pain is usually time limited and when the tissue damage heals, the pain typically resolves. Examples of noniceptive pain are: inflammation (from an infection or arthritic disorder), sprains, bone fractures, burns, bumps, bruises, and obstructions, and myofascial pain and they respond well to treatment with opioids.

Since the peptide affects the wounded area at very low concentrations, it is conceivable that it exerts it effects on the periphery and possibly on the brain. Peripheral opioid receptors are not active in normal tissue but become so within minutes to hours at the onset of inflammation (Stein 1995; Schäfer 1999; Wenk & Honda 1999). It seems that their activity is due to high level of μ-opioid receptor mRNA (Schäfer 1999) and mu-opioid binding sites (Stein 1995; Stein et al. 2003). These facts have demonstrated that opioid receptors are present on sensory nerve terminals before inflammation begins. Thus, peripheral opioid effects must be due to some mechanisms induced by the inflammatory process. Recent studies indicate that opioids gain easier access to neuronal opioid receptors during inflammation because of disruption of the perineurium, an impermeable sheath encasing peripheral nerve fibers (Schäfer 1994). Further, the number of opioid receptors on cutaneous nerve fibers in the inflamed footpad also increases over several days (Stein 1993). These studies demonstrate that inflammation stimulates the axonal transport of opioid receptors to the periphery and increases their number (up-regulation) on peripheral nerve terminals.

The endogenous ligands of peripheral opioid receptors are opioid peptides (endorphin, enkephalin, dynorphin) that have been detected in immune cells within inflamed tissue of animals and humans (Stein 1990, 1993). These opioid peptides occupy their receptors during chronic persistent inflammation.

Pain is basically managed by two classes of drugs. The NSAIDs that block the pain-causing COX enzymes and narcotics, which are the most effective drugs for pain, act on the brain. The peptide has no anti-inflammatory properties and this differentiates it pharmacologically from analgesics in the NSAID's. The exposure of rats to high levels of the peptide did not invoke an abnormal feeding behavior for at least 3 days. Clearly it does not adversely affect brain function under these conditions.

The NSAIDs is in general well tolerated but can induce gastric bleeding and even stroke and heart attack. It is administered orally or topically whereas, the narcotics are administered systemically and their downside is severe side effects. There are growing evidence that some narcotics can be applied peripherally (Stein 1993).

Opioid peptides are administrated directly to the spinal cord but their use is limited because of their short lasting effects, sometimes only a few min. However, some can be given subcutaneously e.g., [Dmt1]DALDA (Neilan 2001; Schiller 2005) or are administrated systemically and get into the brain (Weber et al. 1991). Their metabolism and clearance from the body is complete different routes than small molecules. For example, the liver Cyt P450 is not involved in their breakdown, thus liver toxicity is not an issue. The peptides are degraded by cellular proteases. Peptides at this size (less than 500 Dalton) on one hand lack immunogenicity but on the other hand can be very potent.

The novel peptide may have all the benefits of peptide drugs with high specificity to a specific receptor. A topical application of this peptide analgesic lowers the risk of unforeseen side effects and the small amount needed makes this application safe. Furthermore, its ability to increase the baseline threshold for pain can be used in local anesthetic to block nerve impulses.

### Example 10

The study was conducted with 2 groups of 10 male Sprague Dawley rats, 5-6 weeks old. The rat is a standard species used for the evaluation of potential analgesic properties of a test article. The rats were anesthetized and subjected to a surgical incision to the plantar surface of the right hind paw. Rats were administered the 0.9% saline or SEQ ID NO: 1 at 100 mg/kg 3 hours after surgery and tested for pain responses using von Frey filaments through openings in the cage floor 15-20 minutes later on Day 1 and again on Day 3.

The test article was SEQ ID NO: 1, a white powder, which was delivered in 0.9% saline. The test article formulations were prepared on the day of study and kept at room temperature until dosing. 100 mg/kg at 1 mL/kg=100 mg/mL 1.4 mL of saline added to 141.5 mg of test article, soluble.

Rats were group housed in a room with 12 hour light/12 hour dark at a temperature of 18.9 to 22.2° C.

All animals had access to Harlan Teklad Rodent Diet (certified) ad libitum except during fasting. No contaminants were known to be present in the certified diet at levels that would be expected to interfere with the results of this study. Tap water was available ad libitum, to each animal, via an automatic watering device. No contaminants were known to be present in the water at levels that would be expected to interfere with the results of this study. Study animals were acclimated to their housing for 6 days prior to their first day of dosing.

All animals received for this study were assessed as healthy prior to initiation of the study. Rats were randomly assigned to study groups. The group assignment and dose levels received by each group are illustrated in the table V below.

TABLE V
Group Assignments and Dose Levels

| | Number of | | | Volume |
|---|---|---|---|---|
| | Animals | | | Injected |
| Group | Male | Drug | Dose (mg/kg) | (mL/kg) |
| 1 | 10 | Vehicle | 0 | 1 |
| | | Sterile | | |
| | | saline | | |
| 2 | 10 | SEQ ID | Day 1 (100 mg/kg) | 1 |
| | | NO: 1 | | |

Dosing was administered by subdermal injection adjacent to the wound site 3 hours after the surgery. Each animal received a bolus dose at 1 mL/kg as described in the table.

Animals were weighed, and placed in groups. Animals were anesthetized with 1.8 to 4% isofluorane (delivered via a nose cone) and each received an intramuscular injection of penicillin (30,000 IU) in the triceps muscle after preparation of the foot with betadine and alcohol (SOPs VET-1 and VET-8). A 1 cm long incision of the skin and fascia was made in the plantar aspect (heel, midfoot or distal pad area) of the right hind paw. No underlying muscle was incised. After hemostasis with gentle pressure, the incision was closed with one or two sutures (5-0 silk suture on a taper TF needle). The wound site was covered with a mixture of polymixin B, neomycin, and bacitracin ointment. After surgery, rats were allowed to recover in their cages until behavioral testing. Before the experiment, the rats were placed individually on an elevated plastic mesh floor covered with a clear plastic cage top and allowed to acclimate. Baseline pain behavior was measured as follows: Withdrawal responses to mechanical stimulation was determined using von Frey filaments applied from underneath the cage through openings (12×12 mm in the plastic mesh floor to an area adjacent to the intended incision. Each von Frey filament (Target force of 0.008 g to 300 g) was applied once starting with 0.008 g filament and continuing until a withdrawal response occurred or 300 g force was reached. The median force producing a response, determined from three tests given over a 10-min period was considered the withdrawal threshold. Rats were tested for responses on the day of surgery, 15-20 min after application of the drug and 3 days post-surgery. The test article/vehicle was administered 3 hours after surgery and pain measurements were taken 15-20 min after application of the drug. Rats were euthanized after the measurements were taken on Day 3.

Using Systat v.9.01 software, the data was analyzed by an unpaired t test to compare the vehicle control and the test article groups. Statistical significance was accepted if p≦0.05.

In the saline treated animals, the predose, 3 hour post-surgery and Day 3 post-surgery responses were 34.0, 4.2 and 15.7 g, indicating significant hyperalgesic response immediately after surgery which subsided by Day-3.

In the animals treated with SEQ ID NO: 1 at 100 mg/kg subdermally, the pre-dose, pre-surgery responses were 32.5 g, which was comparable to the saline treated group indicating uniformity of the pain response in both of the groups.

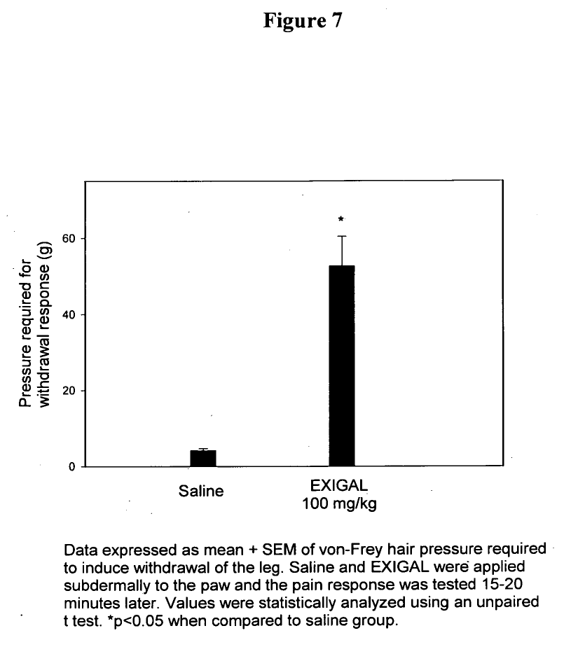

As presented in FIG. 7, the withdrawal response in the 100 mg/kg SEQ ID NO: 1 administered group on Day 1 was 52.7 g compared to the 4.2 g for the saline group at the same time-point, indicating a 1,155% effect which was statistically significant (p≦0.05) compared to the saline treated group.

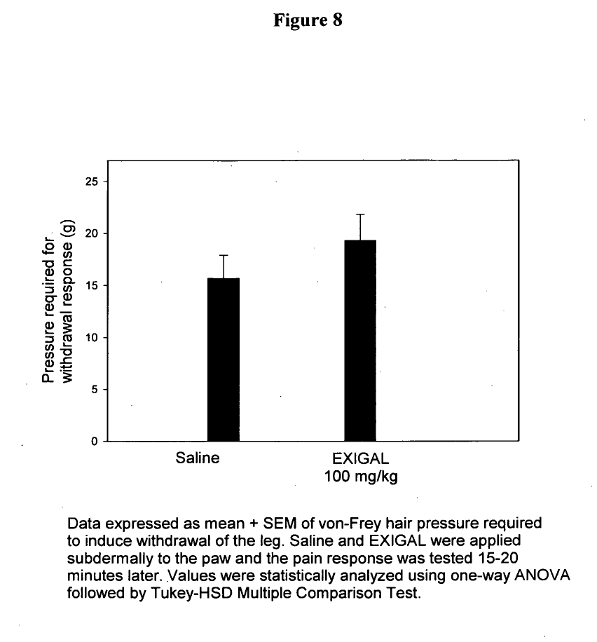

On Day 3, the withdrawal response for the SEQ ID NO: 1 group was 19.3 g, which was comparable to the withdrawal response of 15.7 g observed in the saline group (FIG. 8).

In this assay, SEQ ID NO: 1 administered subdermally at a dose of 100 mg/kg produced a statistically significant (analyzed by unpaired t test) increase in pain threshold in the Brennan model of post-incisional pain in rats.

TABLE VI
Sequence listings.

| | |
|---|---|
| SEQ | |
| ID | |
| NO | Sequence |

| | |
|---|---|
| 1 | FLPS |
| 2 | FLPSEFGVDVDR |
| 3 | KRFLPSEFGVDVDR |
| 4 | KRFFPSEFGLDVDR |
| 5 | KRFLPSEFGFDVDH |
| 6 | KRFFPSEFGNDVDK |
| 7 | KRFFPSEFGTDVDR |
| 8 | KRFLPSEFGMDPPR |
| 9 | KRFLPSEFGMDPAL |
| 10 | RRFLPSEFGLDPDH |
| 11 | KRFLPSEFGMDPDI |
| 12 | KRFFPSEFGNDVDR |
| 13 | KKFYPSEFGNDVDR |
| 14 | VKRFFPSEFGLDVDR |
| 15 | YGGFL |
| 16 | YGGFM |
| 17 | YGGFLRKYPK |
| 18 | YGGPLRKYP |
| 19 | YGGFMTSEKSQTPLVTLFKNAIIKNAYKKGE |
| 20 | YGGFMTSEKSQTPLVT |
| 21 | YGGFLRRIRPKLKWDNQ |
| 22 | YGGFLRRQFKVVT |
| 23 | YPTF |
| 24 | YPFF |
| 25 | Tyr-D-Ala-Gly-N-Methyl-Phe-Gly-ol- |
| 26 | Tyr-D-Pen-Gly-Phe-D-Pen |
| 27 | Tyr-D-Pen-Gly-D-Chloro-Phe-D-Pen |
| 28 | Tyr-D-Pen-Gly-Pen-Pen |
| 29 | Tyr-D-Ser-Gly-Phe-Leu-Thr |
| 30 | Tyr-D-Ala-Gly-Phe-D-Leu |
| 31 | Tyr-Gly-Gly-Phe-Met-Arg-Phe |
| 32 | D-Phe-Cys-Tyr-D-Trp-Orn-Thr-Pen-Thr- |
| 33 | Ac-Arg-Tyr-Arg-Ile-Lys |
| 34 | Tyr-D-Arg-Phe-Lys |
| 35 | Tyr-D-Ala-Gly-N-Methyl-Phe-Gly-ol |
| 36 | Tyr-D-Ala-Gly-Phe-D-Leu-D-Cys |
| 37 | Tyr-D-Ala-Phe-Glu-Val-Val-Pro Gly-amide |
| 38 | Tyr-D-Ala-Phe-Asp-Val-Val-Gly |
| 39 | Tyr-Pro-Methyl-Phe-D-Pro |
| 40 | N,N-diallyl-Tyr-Aib-Aib-Phe-Leu |
| 41 | YPFP |
| 42 | FGGFTGARKSARKLANQ |
| 43 | TEPGLEEVGEIEGQKQLQ |
| 44 | AGEGLNSQFWSLAAPQRF |
| 45 | SQAFLFQPQRF |
| 46 | RPKPQQFFGLM |
| 47 | YPFVEPIP |
| 48 | YPFPGPI |
| 49 | DAEFRHDSGYEVHHQKLVFFAEDVGSNGAIIGLMVGGVVIA |
| 50 | NPFLPS |
| 51 | NPYLPS |
| 52 | NPWLPS |
| 53 | NPHLPS |
| 54 | DPFLPS |
| 55 | DPYLPS |
| 56 | DPWLPS |
| 57 | DPHLPS |
| 58 | FLPPPPSS |
| 59 | YLPPPPSS |
| 60 | WLPPPPSS |
| 61 | HLPPPPSS |
| 62 | GIPYTY |
| 63 | SIPYTY |
| 64 | TIPYTY |
| 65 | GVPYTY |
| 66 | GIPFTY |
| 67 | NIPFTY |
| 68 | GIPFTF |
| 69 | GIPHTY |
| 70 | YTIKAVDD |
| 71 | YTINAVDD |
| 72 | YTIEAVDD |
| 73 | YTINSVDD |
| 74 | YTIRAAND |
| 75 | YTIKTIDD |
| 76 | FTIKAAND |
| 77 | FTIKAVDD |
| 78 | SFGVEASELYPDVKYT |
| 79 | SWGLEASELYPDVKYT |
| 80 | SFGVEATALYPDVKYT |
| 81 | HAVFVNG |
| 82 | HCVEVKG |
| 83 | HSVFVNG |
| 84 | HSAFVKG |
| 85 | HAAFVRG |
| 86 | HAGYIRG |
| 87 | HTAFVKG |
| 88 | HSSYVKG |
| 89 | |
| 90 | DFTSFTIDPSFG |
| 91 | CFAGLFLFVPCLGGCH |
| 92 | HCGGLCPVFLFLGAFC |
| 93 | GAFYAAFC |
| 94 | GFSPDITFSTFD |
| 95 | TLFYGAFC |
| 96 | PLSYGAFF |
| 97 | PLSYGGFY |
| 98 | GAFYGAFM |
| 99 | FFAGYFLP |
| 100 | CFGGYYLP |
| 101 | CFAAYFAG |
| 102 | CFAGYFLT |
| 103 | FFAGYSLP |
| 104 | YFGGYSLP |
| 105 | MFAGYFAG |
| 106 | YFAGYALP |
| 107 | WFADFFLP |
| 108 | PLFYGAFF |
| 109 | PLYYGGFC |
| 110 | TGYIGKFLV |
| 111 | TGYIGKFIV |
| 112 | TGYIGKFVA |
| 113 | TGYIGKYIV |
| 114 | TGYIGKYLV |
| 115 | TGYIGRHV |
| 116 | TGYLGRHV |
| 117 | TGYIGKRIV |
| 118 | TGFIGKRIV |
| 119 | VLFKGIYGT |
| 120 | VIFKGIYGT |
| 121 | AVFKGIYGT |
| 122 | VIYKGIYGT |
| 123 | VLYKGIYGT |
| 124 | VHRGIYGT |
| 125 | VHRGLYGT |
| 126 | TYKVDPYLESAEVGFS |
| 127 | TYKVDPYLESAELGWS |
| 128 | TYKVDPYLATAEVGFS |
| 129 | TYKVDPYLESAEVGFS |
| 130 | TYKVDPYLESAELGWS |
| 131 | TYKVDPYLATAEVGFS |
| 132 | KYILIK |
| 133 | KIYLVK |
| 134 | RIYLIK |
| 135 | RIYLVK |
| 136 | KILYIK |
| 137 | KVLYIK |
| 138 | KVLYIR |
| 139 | IKKEWL |
| 140 | IKNEWL |
| 141 | TKKEWL |
| 142 | LKEWI |
| 143 | IKDEWL |
| 144 | IKGEWL |
| 145 | ILEEWK |
| 146 | LWEKKI |
| 147 | LWENKI |
| 148 | LWEKKT |
| 149 | IWEKL |
| 150 | LWEDKI |
| 151 | LWEGKI |
| 152 | KWEELI |
| 153 | SPLF |
| 154 | RDVDVGFESPLFRK |
| 155 | PFPY |
| 156 | SPLFPN |
| 157 | SPLYPN |
| 158 | SPLWPN |
| 159 | SPLHPN |
| 160 | SPLFPD |
| 161 | SPLYPD |
| 162 | SPLWPD |
| 163 | SPLHPD |
| 164 | SSPPPPLF |
| 165 | SSPPPPLY |
| 166 | SSPPPPLW |
| 167 | SSPPPPLH |
| 168 | GTYPIG |
| 169 | YTYPIS |
| 170 | YTYPIT |
| 171 | YTYPVG |
| 172 | YTFPIG |
| 173 | YTFPIN |
| 174 | FTFPIG |
| 175 | YTHPIG |
| 176 | DDVAKITY |
| 177 | DDVANITY |
| 178 | DDVAEITY |
| 179 | DDVSNITY |
| 180 | DNAARITY |
| 181 | DDITKITY |
| 182 | DNAAKITF |
| 183 | GNVFVAH |
| 184 | GKVFVCH |
| 185 | GNVFVSH |
| 186 | GKVFASH |
| 187 | GVFAAH |
| 188 | GRIYGAH |
| 189 | GKVFATH |
| 190 | GKVYSSH |
| 191 | GKVYSSH |
| 192 | CFAYFAG |
| 193 | |
| 194 | |
| 195 | |
| 196 | |
| 197 | PLFYGAFF |
| 198 | PLYYGGFC |
| 199 | GAFYAAFC |
| 200 | |
| 201 | |
| 202 | |
| 203 | |
| 204 | PLAYGAFY |
| 205 | PLFFDAFW |
| 206 | FFAGYFPL |
| 207 | |
| 208 | VLFKGIYGT |
| 209 | VIFKGIYGT |
| 210 | AVFKGIYTG |
| 211 | VIYKGIYGT |
| 212 | VLYKGIYGT |
| 213 | VHRGIYGT |
| 214 | VHRGLYGT |
| 215 | VIRKGIYGT |
| 216 | VIRKGIFGT |
| 217 | TGYIGKFLV |
| 218 | TGYIGKFIV |
| 219 | TGYIGKFVA |
| 220 | TGYIGKYIV |
| 221 | TGYIGKYLV |
| 222 | TGYIGRHV |
| 223 | TGYLGRHV |
| 224 | KILYIK |
| 225 | KVLYIK |
| 226 | KILYIR |
| 227 | KVLYIR |
| 228 | KIYLIR |
| 229 | KIYLVK |
| 230 | RLYLVK |
| 231 | LWEKKI |
| 232 | LWENKI |
| 233 | LWEKKT |
| 234 | IWEKL |
| 235 | LWEDKI |
| 236 | LWEGKI |
| 237 | KWEELI |
| 238 | IKKEWL |
| 239 | IKNEWL |
| 240 | IKKEL |
| 241 | LKEKL |
| 242 | IKDEWL |
| 243 | IKGEWL |
| 244 | ILEEWK |
| 245 | MDKKSRVLIVGGTGFIGKRIVKASLALGHPTYVLFRPEALSYID |
| | KVQMLISFKQLGAKLLEASLDDHQGLVDVVKQVDVVISAVSG |
| | GLVRHHILDQLKLVEAIKEAGNIKR [symbol] EFGMDPDVVEDPLE |
| | PGNITFIDKRIKVRRAIEAATIPYTYVSSNMFAGFFAGSLAQLQD |
| | APRMMPARDKVLIYGDGNVKGVYVDEDDAGIYIVKSIDDPRT |
| | LNKTVYIRPPMNILSQKEVVEIWERLSGLSLEKIYVSEDQLLNM |
| | KDKSYVEKMARCHLYHFFIKGDLYNFEIGPNATEGTKLYPEV |
| | KYTTMDSYMERYL |
| 246 | MGESKRTEKTRVLVVGATGYIGKRIVRACLAEGHETYVLQRP |
| | EIGLEIEKVQLFLSFKKLGARIVEGSFSDHQSLVSAVKLVDVVV |
| | SAMSGVHFRSHNILVQLKLVEAIKEAGNVKR [symbol] EFGMDPPR |
| | MGHALPPGRETFDQKMERQAIEAAGIPYTYVVGACFAAYFAG |
| | NLSQMVTLLPPKEKVNIYGDGNVKVVFADEDDIAKYTAKTLN |
| | DPRTLNKTVNIRPPDNVLTQLELVQIWEKLTGKELEKTNINAQ |
| | DFLANIEQMEIPHQAGIGHFYHIFYEGCLTDHEVGEDEEASSLY |
| | PDVKYKRMDDYLRMFL |
| 247 | MATEKSKILVIGGTGYIGKFLVEASAKAGHSTFALVREATLSD |
| | PVKGKTVQSFKDLGVTILHGDLNDHESLVKAIKQVDVVISTVG |
| | SMQILDQTKIISAIKEAGNVKR [symbol] EFGVDVDRTSAVEPAKSA |
| | FAGKIQIRRTIEAEGIPYTYAVTGCFGGYYLPTLVQFEPGLTSPP |
| | RDKVTILGDGNAKAVINKEEDIAAYTIKAVDDPRTLNKILYIKP |
| | SNNTLSMNEIVTLWEKKIGKSLEKTHLPEEQLLKSIQESPIPINV |
| | VLSINHAVFVNG |
| | DTNISIEPSFGVEASELYPDVKYTSVDEYLSYFA |
| 248 | YGGFLRRQFKVVT |
| 249 | KYPKRSSEVAGEGDGDSMGHEDLYKRYGGFLRRIRPKLKWD |
| | NQKRYGGFLRRQFKVVTRSQEDPNAYSGELFDA |
| 250 | MAWQGLVLAACLLMFPSTTADCLSRCSLCAVKTQDGPKPINP |
| | LICSLQCQAALLPSEEWERCQSFSFFTPSTLGLNDKEDLGSKSV |
| | GEGPYSELAKLSGSFLKELEKSK [symbol] ISTKENTLSKSLEEKL |
| | RGLSDGFREGAESELMRDAQLNDGAMETGTLYLAEEDPKEQ |
| | VKR [symbol] [symbol] KRSSEVAGEGDGDSMGHEDLYKR [symbol] |
| | [symbol] [symbol] [symbol] [symbol] [symbol] [symbol] |
| 251 | ttt ctg ccc tca |
| 252 | ttt ctg ccc tca gaa ttt gga gta gac gta gac aga |
| 253 | MVKKIANDVSNKLFPLPKGFGDFVGIEDHIKAIKSILCLESKEA |
| | RIMVGIWGQSGIGKSTIGRALFSQLSSQFHHRAFITYKSTSGSD |
| | VSGMKLSWEKELLSEILGQKDIKIDHFGVVEQRLKHKKVLILL |
| | DDVDNLEFLKTLVGKAEWFGSGSRIIVITQDKQLLKAHEIDLV |
| | YEVELPSQGLALKMISQYAFGKDSPPDDFKELAFEVAELVGSL |
| | PLGLSVLGSSLKGRDKDEWVKMMPRLRNDSDDKIEETLRVGY |
| | DRLNKKNRDNVKELLEDDVGLTMLADKSLIRITPDGDIEMHN |
| | LLEKLGREIDRAKSKGNPAKRQFLTNFEDIQEVVTEKTGTETV |
| | LGIRVPPTVLFSTRPLLVINEFSFKGMQIGLWSKIDLPQGLVYLP |
| | LKLKLLKWNYCPLKSLPSTFKAEYLVNLIMKYSKLEKLWEGT |
| | LPLGSLKXMDLGCSNNLKEIFDLSLAINLEELNLSKCESLVTLP |
| | SSIQNAIKLRTLYCSGVLLIDLKSLEGMCNLEYLSVDWSSMEG |
| | TQGLIYLPRKLKRLWWDYCPVKRLPSNFKAEYLVELRMENSD |
| | LEKLWDGTQPLGSLKEMYLHGSKYLKEIPDLSLAINLERLYLF |
| | GCESLVTLPSSIQNATKLINLDMRDCKKLESFPTDLNLESLEYL |
| | NLTGCPNLRNFPAIKMGCSYFEILQDRNEIEVEDCFWNKNLPA |
| | GLDYLDCLMRCMPCEFRPEYLTFLDVSGCKHEKLWEGIQIHA |
| | LLDGYELAGHLDGSIETPAPTLTTNNVVSANPQYTLWKRQDR |
| | LIFSALIGAISPPVQPLVSRATKASQIWKTLTNTYAKSSYDHIKQ |
| | LRTQIKQLKKGTKTIDEYVLSHTTLLDQLAILGKPMEHEEQVE |
| | RILEGLPEDYKTVVDQIEGKDNTPSITEIHERLINHEAKLLSTAA |
| | LSSSSLPMSANVAQQRHHNNNRNNNQNKNRTQGNTYTNNW |
| | QPSANNKSGQRPPKPYLGKCQICNVQGHSARRCPQLQAMQPS |
| | SSSSASTFTPWQPRANLAMGAPYTANNWLLDSGATHHITSDL |
| | NALALHQPYNGDDVMIADGTSLKITKTGSTFLPSNARDLTLNK |
| | VLYVPDIQKNLVSVYRLCNTNQVSVEFFPASFQVKDLNTGTLL |
| | LQGRTKDELYEWPVTNPKATALFTTPSPKTTLSSWHSRLGHPS |
| | SSILNTLISKFSLPVSVSASNKLACSDCFINKSHKLPFSISSIKSTS |
| | PLEYIFSDVWMSPILSPDNYKYYLQKSQVKSTFIAFKALVENRF |
| | QAKIRTLYSDNGGEFIALREFLVSNGISHLTSPPHTPEHNGLSER |
| | KHRHIVETGLTLLTQASVPREYWPYAFAAAVYLINRMPTPVLS |
| | MESPFQKLFGSKPNYERLRVFGCLCFPWLRPYTHNKLEERSRR |
| | CVFLGYSTQTAYLCFDVEHKRLYTSRHVVFDEASFPFSNLTSQ |
| | NSLPTVTFEQSSSPLVTPILSSSSVLPSCLSSPCTVLHQQQPPVTT |
| | PNSPHSSQPTTSPAPLSPHRSTTMDFQVPQPTAPNENGPEPEAQ |
| | SPPIGPLSNPTHEAFIGPLPNPNRNPTNEIEPTPAPHPKPVKPTTT |
| | TTTPNRTTVSDASHQPTAPQQNQHNMKTRAKNNIKKPNTKFS |
| | LTATLPNRSPSEPTNVTQALKDKKWRFAMSDEFDAQQRNHT |
| | WDLVPHESQLLVGCKWVFKLKYLPNGAIDKYKARLVAKGFN |
| | QQYGVDYAETFSPVIKSTTIRLVLDVAVKKDWEIKQLDVNNA |
| | FLQGTLTEEVYMAQPPGFIDKDRPTHVCRLRKAIYGLKQAPRA |
| | WYMELKQHLFNIGFVNSLSDASLFIYWSDKSSIDAVLTSLAER |
| | FSIKDPTDLHYFLGIEATRTKQGLHLMQRKYIKDLLAKHNMA |
| | DAKPVLTPLPTSPKLTLHGGTKLNDASEYRSVVGSLQYLAFTR |
| | PDIAYAVNRLSQLMPQPTEDHWQAAKRVLRYLAGTSTHDWA |
| | GDSDDYVSTNAYVIYLGKNPISWSSKKQRGVARSSTESEYRA |
| | VANAASEVKWLCSLLSKLHIRLPIRPSIFCDNIGATYLCANPVF |
| | HSRMKHIAIDYHFVRNMIQSGALRVSHVSTRDQLADALTKPLS |
| | RAHFQSARFKIGVRQLPPS |
| 254 | MSTSSLRRQMKNIVHNYSEAEIKVREATSNDPWGPSSSLMSEI |
| | ADLTYNVVAFSEIMSMIWKRLNDHGKNWRHVYKAMTLMEY |
| | LIKTGSERVSQQCKENMYAVQTLKDFQYVDRDGKDQGVNVR |
| | EKAKQLVALLRDEDRLREERAHALKTKEKLAQTATASSAAVG |
| | SGPPPEAEQAWPQSSGEEELQLQLALAMSKEEADQPPSCGPED |
| | DVQLQLALSLSREEHDKEERIRRGDDLRLQMAIEESKRETGGK |
| | EESSLMDLADVFTTPAPPQASDPWGGPASVPTAVPVAAAASD |
| | PWGAPAVPPAADPWGGAAPTPASGDPWRLPAAPTGPSVDPWG |
| | GTPAPAAGEGPTSDPWGSADGGAPVSGPPSSDPWAPAPAFSDP |
| | WGGSPAKPSSNGTAVGGFDTEPDEFSDFDRLRTALPTSGSSTG |
| | ELELLAGEVPARSPGAFDMSGVGGSLAESVGSPPPAATPTPTPP |
| | TRKTPESFLGPNAALVDLDSLVSRPGPTPPGAKASNP [symbol] GAP |
| | ATGPSVTNPFQPAPPATLTLNQLRLSPVPPVPGGAPPTYISPLGG |
| | GPGLPPMMPPGPPAPNTNPFLL |
| 255 | MEPPLPVGAQPLATVEGMEMKGPLREPCALTLAQRNGQYELII |
| | QLHEKEQHVQDIIPINSHFRCVQEAEETLLIDIASNSGCKIRVQG |
| | DWIRERRFEIPDEEHCLKFLSAVLAAQKAQSQLLVPEQKDSSS |
| | WYQKLDTKDKPSVFSGLLGFEDNFSSMNLDKKINSQNQPTGIH |
| | REPPPPPFSVNKMLPREKEASNKEQPKVTNTMRKLFVPNTQSG |
| | QREGLIKHILAKREKEYVNIQTFRFFVGTWNVNGQSPDSGLEP |
| | WLNCDPNPPDIYCIGFQELDLSTEAFFYFESVKEQEWSMAVER |
| | GLHSKAKYKKVQLVRLVGMMLLIFARKDQCRYIRDIATETVG |
| | TGIMGKMGNKGGVAVRFVFHNTTFCIVNSHLAAHVEDFERRN |
| | QDYKDICARMSFVVPNQTLPQLNIMKHEVVIWGDLNYRLCMP |
| | DANVKSLINKKDLQRLLKFDQLNIQRTQKKAFVDFNEGEIKF |
| | IPTYKYDSKTDRWDSSGKCRVPAWCDRILWRGTNYNQLNYR |
| | SHMELKTSDHKPVSALFHIGVKVVDERRYRKVFEDSVRIMDR |
| | MEND [symbol] LELSREFVFENVKFRQLQKEKFQISNNGQVPCHF |
| | SFIPKLNDSQYCKPWLRAEPFEGYLEPNETVDISLDVYVSKDS |
| | VTILNSGEDKIEDILVLHLDRGKDYFLTISGNYLPSCFGTSLEAL |
| | CRMKRPIREVPVTKLIDLEEDSFLEKEKSLLQMVPLDEGASERP |
| | LQVPKLEIWLLVDHLFKYACHQEDLFQTPGMQEELQQIIDCLDT |
| | SIPETIPGSNHSVAEALLIFLEALPEPVICYELYQRCLDSAYDPRI |
| | CRQVISQLPRCHRNVFRYLMAFLRELLKFSEYNSVNANMIATL |
| | FTSLLLRPPPNLMARQTPSDRQRAIQFLLGFLLGSEED |
| 256 | MSESGNTTSMPGCGRMCALRSTWSKRAFLVACKDGALTSDG |
| | RCPQYGCGALVSITKGVQQPKKTASAKVVKCLCWVQPARWC |
| | EKHSKGPASPNGSVTTKRSNSARAAPAPLPYKKQTGDVVVTV |
| | GPLELVYPALVSEELPTPVAATPTKVEEVPIPELPLWLALPAWM |
| | VEQPYAATPEVLVLTQREEFALLKKRLTRKGKLLQRRATHAR |
| | FEARAALARVRAATQRKVEEVTALVIKGRRILAAHQLLRELEE |
| | VAPLSQAQEQLVASSCAAAAARQEECASFLRRAKAWRKSISA |
| | TPPVAATAVASKVSATMPWAHLGLSLGGLLAVPTLDGTLGA |
| | KQWNAKTIATWVLKPVVSCVQSVHAKVRDWLHSQPEVGVT |
| | NTKVPLVLPEVCLGVLSPPSLSEEIVDNPQETSQSGIWHPEMGV |
| | RNIYVFHDDSWETSPEEDENYTYTFSRQCGIPYLLVEGRGAEE |
| | RKNTILGWDFSLHNDGFEFLPSPEEGYTKELVTPVALEEEDKY |
| | STASSCGFFSLDDVSSAITIQCPGLLSADADVHFFDGPGYRCSS |
| | RPRDFRPPVVRGCDYESRVKASIQRKIENPLQERFITVLREKRK |
| | KNKKKEFHSFSACFAFKRKQIQWPPTPNEMVNEWEEYCIAQA |
| | WLPFEVVVTDEIEDVTPLYPGGRDYNCNSQLLFPLAPLSTVYC |
| | DDSCFHPNDGWTTDGNGKHFRLSPQFVLPDVPIPIVHRVTRQL |
| | PQFLYDLGIGDLTCNSGYQAENLQEEIQERMEDRSEEKPVPSL |
| | DTLISKLSKRSTKVKGAGENRYADRHSLTEKAIFHQPGALSRM |
| | RSGKEKTIVAANHNSDQISVRMAECGKPVFTPLPRMSDEMLR |
| | KFLEKGLGSTSTVALDIFIQSHIPQGMPTVAFVNVMDTRIEDPL |
| | YSSLCGSYIDLGRDRAKTLCLPLVNFPMSKLAEDVDDVLNGL |
| | MLCTHFQDSTKFGVGKPAFQYGTLEFQEFKPSAYSDFSRVRD |
| | NWDAIAKQQNTPNDRILAGFSVLGAVSQAYNQALPVFKSVEL |
| | VAPPKRKPVVATFQNPTTLGRSNTTRSFRMPTMDLPRSTGRDA |
| | PIPIVHRRNNNDVHFDEATPARFSTCDSGLVADTTLAFAKMYQ |
| | CKKDAKAGHVLATIDIQECVFEDNRRVALDWLAHGLASFKY |
| | DLQLTVDSNPFVGVTLGITVDAFDRLLPQISDEVIAVPLAFQLP |
| | TYLFPISKKGTFTQTIDFAAIAGYNFFPHVAAFGRPKIIVYIVSD |
| | NDLPASDTWMCLVELHMTRLESSTLACSPTLVLPQAFGGDLP |
| | LDLWRGPYTFPLGGGTKRLSTSLDIGTSTTTVSGWRTVSPAAY |
| | ALFLQGHGGSLVGEVVHTGSAAVSCALHLCISFGGAPPTLEEA |
| | LVFPGFRLPSGEGKFHIKVQTPYGRLSTLTPDCALYVYLAGGPI |
| | AVAPMSVPYQFCIHLERLVDDGAPPRTIGLIREFNWATINNFKS |
| | DDITFAIPARLSDLVLTCGDVTMSTNPLALLIGSCGFFRGNLTV |
| | VLEWATFLKAGDKEGTVQLTTCRGMINNVKGVRNAIQKKVV |
| | NLSLVGSVSRYLNVGDFTGFAQSGGQVGYDEIFLEFSTNKAKQ |
| | IRYLNINVELDENFELYGRTIIPLKNTAPAFASTSSAPNES |
| 257 | KFLPS |
| 258 | FLPSI |
| 259 | RFLPS |
| 260 | FLPSE |
| 261 | SFLK |
| 262 | WERC |
| 263 | FSFFTP |
| 264 | FFNP |
| 265 | FPST |
| 266 | SFLG |
| 267 | YSEL |
| 268 | SYLG |
| 269 | SWLG |
| 270 | FFTPS |
| 271 | SFLSFFTPS |
| 272 | NPFQP |
| 273 | MSFLK |
| 274 | FLPPPS |
| 275 | FLPPS |
| 276 | MFPST |
| 277 | PLPPPPPPS |
| 278 | FLPPPPPS |
| 279 | FGGFIM |
| 280 | KLFS |
| 281 | CREW |
| 282 | PTFFSF |
| 283 | PNFF |
| 284 | TSPF |
| 285 | GLFS |
| 286 | LESY |
| 287 | GLYS |
| 288 | GLWS |
| 289 | SPTFF |
| 290 | SPTFFSLFS |
| 291 | PQFPN |
| 292 | KLFSM |
| 293 | SPPPPLF |
| 294 | SPPLF |
| 295 | TSPFM |
| 296 | SPPPPPPPLF |
| 297 | SPPPPPPLF |
| 298 | MIFGGF |
| 299 | MAWQGLVLAACLLMFPSTTADCLSRCSLCAVKTQDGPKPINP |
| | LICSLQCQAALLPSEEWERCQSFLSFFTPSTLGLNDKEDLGSKS |
| | VGEGPYSELAKLSGSFLKELEKSKFLPSISTKENTLSKSLEEKLR |
| | GLSDGFREGAESELMRDAQLNDGAMETGTLYLAEEDPKEQVKR |
| 300 | MARFLTLCTWLLLLGPGLLATVRAECSQDCATCSYRLVRPADI |
| | NFLACVMECEGKLPSLKIWETCKELLQLSKPELPQDGTSTLRE |
| | NSKPEESHLLAKRY |
| 301 | MARFLTLCTWLLLLGPGLLATVRAECSQDCATCSYRLVRPADI |
| | NFLACVMECEGKLPSLKIWETCKELLQLSKPELPQDGTSTLRE |
| | NSKPEESHLLAKRYGGFMKRYGGFMKKMDELYPMEPEEEAN |
| | GSEILAKRYGGFMKKDAEEDDSLANSSDLLKELLETGDNRERS |
| | HHQDGSDNEEENSKRYGGFMRGLKRSPQLEDEAKELQKRYG |
| | GFMRRVGRPEWWMDYQKRYGGFLKRFAEALPSDEEGESYSK |
| | EVPEMEKRYGGFMRF |
| 302 | MPRSCCSRSGALLLALLLQASMEVRGWCLESSQCQDLTTESN |
| | LLECIRACKPDLSAETPMFPGNGDEQPLTENPRKYVMGHFRW |
| | DRFGRRNSSSSGSSGAGQKREDVSAGEDCGPLPEGGPEPRSDG |
| | AKPGPREGKRSYSMEHFRWGKPVGKKRRPVKVYPNGAEDES |
| | AEAFPLEFKRELTGQRLREGDGPDGPADDGAGAQADLEHSLL |
| | VAAEKKDEGPYRMEHFRWGSPPKDKRYGGFMTSEKSQTPL |
| | VTLFKNAIIKNAYKKGE |
| 303 | MPRSCCSRSGALLLALLLQASMEVRGWCLESSQCQDLTTESN |
| | LLECIRACKPDLSAETPMFPGNGDEQPLTENPRKYVMGHFRW |
| | DRFGRRNSSSSGSSGAGQKREDVSAGEDCGPLPEGGPEPRSDG |
| | AKPGPREGKRSYSMEHFRWGKPVGKKRRPVKVYPNGAEDES |
| | AEAFPLEFKRELTGQRLREGDGPDGPADDGAGAQADLEHSLL |
| | VAAEKKDEGPYRMEHFRWGSPPKDKR |
| 304 | FLPSPLF |
| 305 | FLPSSPLF |
| 306 | PFLP |
| 307 | SLFP |
| 308 | FPSA |
| 309 | |
| 310 | AFLP |
| 311 | TLPF |
| 312 | PPLF |
| 313 | FPSP |
| 314 | FLVS |
| 315 | PLFP |
| 316 | FLFS |
| 317 | LFSF |
| 318 | MFTS |
| 319 | PSLF |
| 320 | PSSF |
| 321 | FTPS |
| 322 | FLSF |
| 323 | LPSF |
| 324 | FKPS |
| 325 | FLSP |
| 326 | FPLS |
| 327 | FPSL |
| 328 | FSPL |
| 329 | FSLP |
| 330 | LFPS |
| 331 | LFSP |
| 332 | LPFS |
| 333 | LSPF |
| 334 | LSFP |
| 335 | PFLS |
| 336 | PFSL |
| 337 | PLSF |
| 338 | PLFS |
| 339 | PSFL |
| 340 | SFLP |
| 341 | SFPL |
| 342 | SPFL |
| 343 | SLPF |
| 344 | FLP |
| 345 | FPL |
| 346 | PLF |
| 347 | LPF |
| 348 | LFP |
| 349 | PFL |
| 350 | FLPSDFPPSV |
| 351 | TPPAYRPPNAPILFLPSDFFPSV-NH2 |
| 352 | TPPAYRPPNAPILFLPSDFEPSV |
| 353 | TPPAYRPPNAPILAAAFLPSDFFPSV-NH2 |
| 354 | TPPAYRPPNAPILAAAFLPSDFFPSV |
| 355 | AC-QYIKANSKFIGITEAAAFLPSDFFPSV |
| 356 | QYIKANSKFIGITEAAAFLPSDFFPSV |
| 357 | KKFLPSEFGHD |
| 358 | FLPSEFGHDVDR |
| 359 | ATVELLSFLPSDFPPSV |
| 360 | TVELLSFLPSDFFPSV |
| 361 | VELLSFLPSDFFPSV |
| 362 | ELLSFLPSDFFPSV |
| 363 | LLSFLPSDFFPSV |
| 364 | LSFLPSDFFPSV |
| 365 | FLPSDFFPSV |
| 366 | FLPSDFFPS |
| 367 | SFLPSDFFP |
| 368 | LSFLPSDFF |
| 369 | LLSFLPSDF |
| 370 | ELLSFLPSD |
| 371 | VELLSFLPS |

1. An isolated polypeptide consisting essentially of FLPS (SEQ ID NO: 1) or an analog thereof, but specifically excluding all of SEQ ID NOS: 350-371.

2. A fragment of any one of SEQ ID NOS: 245-247 and 250, 253-256 wherein said fragment comprises FLPS (SEQ ID NO: 1) or an analog thereof.

3. An isolated polypeptide comprising FLPS (SEQ ID NO: 1) having less than 100 or 50 or 30 amino acid residues or an analog thereof, but specifically excluding all of SEQ ID NOS: 350-371.

4. An isolated polypeptide consisting of FLPS (SEQ ID NO: 1) and 0, 1, 2, 3, 4, 5, 6, 7, 8, 9, or 10 additional amino acids, but specifically excluding all of SEQ ID NOS: 350-371.

5. A method of treating pain by administering to an animal in need thereof a composition of claim 1, 2, 3, or 4
